SEQUENCE LISTING (53 sequences)

SEQ ID NO: 1 - DNA, 702 bp, Artificial Sequence
  Human TK1 mutM73AA75DD83AG9095A
atgagctgcattaacctgcccactgtgctgcccggctcccccagcaagacccgggggcagatccaggtgattctcgggccgatgttctcaggaaaaagcacagagttgatgagacgcgtccgtcgcttccagattgctcagtacaagtgcctggtgatcaagtatgccaaagacactcgctacagcagcagcttctgcacacatgaccggaacaccgccgaggacctgcccgcctgcctgctccgagccgtggcccaggaggccctggctgtggctgtcatagctatcgacgaggggcagtttttccctgacatcgtggagttctgcgaggccatggccaacgccgggaagaccgtaattgtggctgcactggatgggaccttccagaggaagccatttggggccatcctgaacctggtgccgctggccgagagcgtggtgaagctgacggcggtgtgcatggagtgcttccgggaagccgcctataccaagaggctcggcacagagaaggaggtcgaggtgattgggggagcagacaagtaccactccgtgtgtcggctctgctacttcaagaaggcctcaggccagcctgccgggccggacaacaaagagaactgcccagtgccaggaaagccaggggaagccgtggctgccaggaagctctttgccccacagcagattctgcaatggatccagacatga

SEQ ID NO: 2 - protein, 233 aa, Artificial Sequence
  Human TK1 mutM73AA75DD83AG9095A
MSCINLPTVLPGSPSKTRGQIQVILGPMFSGKSTELMRRVRRFQIAQYKCLVIKYAKDTRYSSSFCTHDRNTAEDLPACLLRAVAQEALAVAVIAIDEGQFFPDIVEFCEAMANAGKTVIVAALDGTFQRKPFGAILNLVPLAESVVKLTAVCMECFREAAYTKRLGTEKEVEVIGGADKYHSVCRLCYFKKASGQPAGPDNKENCPVPGRPGEAVAARKLFAPQQILQWIQT

SEQ ID NO: 3 - DNA, 702 bp, Artificial Sequence
  Human TK1 mutM73AA75DD83AG90A
atgagctgcattaacctgcccactgtgctgcccggctcccccagcaagacccgggggcagatccaggtgattctcgggccgatgttctcaggaaaaagcacagagttgatgagacgcgtccgtcgcttccagattgctcagtacaagtgcctggtgatcaagtatgccaaagacactcgctacagcagcagcttctgcacacatgaccggaacaccgccgaggacctgcccgcctgcctgctccgagccgtggcccaggaggccctggctgtggctgtcataggcatcgacgaggggcagtttttccctgacatcgtggagttctgcgaggccatggccaacgccgggaagaccgtaattgtggctgcactggatgggaccttccagaggaagccatttggggccatcctgaacctggtgccgctggccgagagcgtggtgaagctgacggcggtgtgcatggagtgcttccgggaagccgcctataccaagaggctcggcacagagaaggaggtcgaggtgattgggggagcagacaagtaccactccgtgtgtcggctctgctacttcaagaaggcctcaggccagcctgccgggccggacaacaaagagaactgcccagtgccaggaaagccaggggaagccgtggctgccaggaagctctttgccccacagcagattctgcaatggatccagacatga

SEQ ID NO: 4 - protein, 233 aa, Artificial Sequence
  Human TK1 mutM73AA75DD83AG90A
MSCINLPTVLPGSPSKTRGQIQVILGPMFSGKSTELMRRVRRFQIAQYKCLVIKYAKDTRYSSSFCTHDRNTAEDLPACLLRAVAQEALAVAVIGIDEGQFFPDIVEFCEAMANAGKTVIVAALDGTFQRKPFGAILNLVPLAESVVKLTAVCMECFREAAYTKRLGTEKEVEVIGGADKYHSVCRLCYFKKASGQPAGPDNKENCPVPGRPGEAVAARKLFAPQQILQWIQT

SEQ ID NO: 5 - RNA, 702 nt, Artificial Sequence
  Human TK1 mutV84EG90A
atgagctgcattaacctgcccactgtgctgcccggctcccccagcaagacccgggggcagatccaggtgattctcgggccgatgttctcaggaaaaagcacagagttgatgagacgcgtccgtcgcttccagattgctcagtacaagtgcctggtgatcaagtatgccaaagacactcgctacagcagcagcttctgcacacatgaccggaacaccatggaggcgctgcccgcctgcctgctccgagacgaggcccaggaggccctggctgtggctgtcataggcatcgacgaggggcagtttttccctgacatcgtggagttctgcgaggccatggccaacgccgggaagaccgtaattgtggctgcactggatgggaccttccagaggaagccatttggggccatcctgaacctggtgccgctggccgagagcgtggtgaagctgacggcggtgtgcatggagtgcttccgggaagccgcctataccaagaggctcggcacagagaaggaggtcgaggtgattgggggagcagacaagtaccactccgtgtgtcggctctgctacttcaagaaggcctcaggccagcctgccgggccggacaacaaagagaactgcccagtgccaggaaagccaggggaagccgtggctgccaggaagctctttgccccacagcagattctgcaatggatccagacatga

SEQ ID NO: 6 - protein, 233 aa, Artificial Sequence
  Human TK1 mutV84EG90A
MSCINLPTVLPGSPSKTRGQIQVILGPMFSGKSTELMRRVRRFQIAQYKCLVIKYAKDTRYSSSFCTHDRNTMEALPACLLRDEAQEALAVAVIGIDEGQFFPDIVEFCEAMANAGKTVIVAALDGTFQRKPFGAILNLVPLAESVVKLTAVCMECFREAAYTKRLGTEKEVEVIGGADKYHSVCRLCYFKKASGQPAGPDNKENCPVPGRPGEAVAARKLFAPQQILQWIQT

SEQ ID NO: 7 - DNA, 702 bp, Artificial Sequence
  Human TK1 mutM73AA75DG9095A
atgagctgcattaacctgcccactgtgctgcccggctcccccagcaagacccgggggcagatccaggtgattctcgggccgatgttctcaggaaaaagcacagagttgatgagacgcgtccgtcgcttccagattgctcagtacaagtgcctggtgatcaagtatgccaaagacactcgctacagcagcagcttctgcacacatgaccggaacaccgccgaggacctgcccgcctgcctgctccgagacgtggcccaggaggccctggctgtggctgtcatagctatcgacgaggggcagtttttccctgacatcgtggagttctgcgaggccatggccaacgccgggaagaccgtaattgtggctgcactggatgggaccttccagaggaagccatttggggccatcctgaacctggtgccgctggccgagagcgtggtgaagctgacggcggtgtgcatggagtgcttccgggaagccgcctataccaagaggctcggcacagagaaggaggtcgaggtgattgggggagcagacaagtaccactccgtgtgtcggctctgctacttcaagaaggcctcaggccagcctgccgggccggacaacaaagagaactgcccagtgccaggaaagccaggggaagccgtggctgccaggaagctctttgccccacagcagattctgcaatggatccagacatga

SEQ ID NO: 8 - protein, 233 aa, Artificial Sequence
  Human TK1 mutM73AA75DG9095A
MSCINLPTVLPGSPSKTRGQIQVILGPMFSGKSTELMRRVRRFQIAQYKCLVIKYAKDTRYSSSFCTHDRNTAEDLPACLLRDVAQEALAVAVIAIDEGQFFPDIVEFCEAMANAGKTVIVAALDGTFQRKPFGAILNLVPLAESVVKLTAVCMECFREAAYTKRLGTEKEVEVIGGADKYHSVCRLCYFKKASGQPAGPDNKENCPVPGRPGEAVAARKLFAPQQILQWIQT

SEQ ID NO: 9 - DNA, 702 bp, Artificial Sequence
  Human TK1 mutM73AA75DG90A
atgagctgcattaacctgcccactgtgctgcccggctcccccagcaagacccgggggcagatccaggtgattctcgggccgatgttctcaggaaaaagcacagagttgatgagacgcgtccgtcgcttccagattgctcagtacaagtgcctggtgatcaagtatgccaaagacactcgctacagcagcagcttctgcacacatgaccggaacaccgccgaggacctgcccgcctgcctgctccgagacgtggcccaggaggccctggctgtggctgtcataggcatcgacgaggggcagtttttccctgacatcgtggagttctgcgaggccatggccaacgccgggaagaccgtaattgtggctgcactggatgggaccttccagaggaagccatttggggccatcctgaacctggtgccgctggccgagagcgtggtgaagctgacggcggtgtgcatggagtgcttccgggaagccgcctataccaagaggctcggcacagagaaggaggtcgaggtgattgggggagcagacaagtaccactccgtgtgtcggctctgctacttcaagaaggcctcaggccagcctgccgggccggacaacaaagagaactgcccagtgccaggaaagccaggggaagccgtggctgccaggaagctctttgccccacagcagattctgcaatggatccagacatga

SEQ ID NO: 10 - protein, 233 aa, Artificial Sequence
  Human TK1 mutM73AA75DG90A
MSCINLPTVLPGSPSKTRGQIQVILGPMFSGKSTELMRRVRRFQIAQYKCLVIKYAKDTRYSSSFCTHDRNTAEDLPACLLRDVAQEALAVAVIGIDEGQFFPDIVEFCEAMANAGKTVIVAALDGTFQRKPFGAILNLVPLAESVVKLTAVCMECFREAAYTKRLGTEKEVEVIGGADKYHSVCRLCYFKKASGQPAGPDNKENCPVPGRPGEAVAARKLFAPQQILQWIQT

SEQ ID NO: 11 - DNA, 702 bp, Artificial Sequence
  Human TK1 mutG90A
atgagctgcattaacctgcccactgtgctgcccggctcccccagcaagacccgggggcagatccaggtgattctcgggccgatgttctcaggaaaaagcacagagttgatgagacgcgtccgtcgcttccagattgctcagtacaagtgcctggtgatcaagtatgccaaagacactcgctacagcagcagcttctgcacacatgaccggaacaccatggaggcgctgcccgcctgcctgctccgagacgtggcccaggaggccctggctgtggctgtcataggcatcgacgaggggcagtttttccctgacatcgtggagttctgcgaggccatggccaacgccgggaagaccgtaattgtggctgcactggatgggaccttccagaggaagccatttggggccatcctgaacctggtgccgctggccgagagcgtggtgaagctgacggcggtgtgcatggagtgcttccgggaagccgcctataccaagaggctcggcacagagaaggaggtcgaggtgattgggggagcagacaagtaccactccgtgtgtcggctctgctacttcaagaaggcctcaggccagcctgccgggccggacaacaaagagaactgcccagtgccaggaaagccaggggaagccgtggctgccaggaagctctttgccccacagcagattctgcaatggatccagacatga

SEQ ID NO: 12 - protein, 233 aa, Artificial Sequence
  Human TK1 mutG90A
MSCINLPTVLPGSPSKTRGQIQVILGPMFSGKSTELMRRVRRFQIAQYKCLVIKYAKDTRYSSSFCTHDRNTMEALPACLLRDVAQEALAVAVIGIDEGQFFPDIVEFCEAMANAGKTVIVAALDGTFQRKPFGAILNLVPLAESVVKLTAVCMECFREAAYTKRLGTEKEVEVIGGADKYHSVCRLCYFKKASGQPAGPDNKENCPVPGRPGEAVAARKLFAPQQILQWIQT

SEQ ID NO: 13 - DNA, 702 bp, Artificial Sequence
  Human TK1 mutA75DG90E
atgagctgcattaacctgcccactgtgctgcccggctcccccagcaagacccgggggcagatccaggtgattctcgggccgatgttctcaggaaaaagcacagagttgatgagacgcgtccgtcgcttccagattgctcagtacaagtgcctggtgatcaagtatgccaaagacactcgctacagcagcagcttctgcacacatgaccggaacaccatggaggacctgcccgcctgcctgctccgagacgtggcccaggaggccctggaggtggctgtcataggcatcgacgaggggcagtttttccctgacatcgtggagttctgcgaggccatggccaacgccgggaagaccgtaattgtggctgcactggatgggaccttccagaggaagccatttggggccatcctgaacctggtgccgctggccgagagcgtggtgaagctgacggcggtgtgcatggagtgcttccgggaagccgcctataccaagaggctcggcacagagaaggaggtcgaggtgattgggggagcagacaagtaccactccgtgtgtcggctctgctacttcaagaaggcctcaggccagcctgccgggccggacaacaaagagaactgcccagtgccaggaaagccaggggaagccgtggctgccaggaagctctttgccccacagcagattctgcaatggatccagacatga

SEQ ID NO: 14 - protein, 233 aa, Artificial Sequence
  Human TK1 mutA75DG90E
MSCINLPTVLPGSPSKTRGQIQVILGPMFSGKSTELMRRVRRFQIAQYKCLVIKYAKDTRYSSSFCTHDRNTMEDLPACLLRDVAQEALEVAVIGIDEGQFFPDIVEFCEAMANAGKTVIVAALDGTFQRKPFGAILNLVPLAESVVKLTAVCMECFREAAYTKRLGTEKEVEVIGGADKYHSVCRLCYFKKASGQPAGPDNKENCPVPGRPGEAVAARKLFAPQQILQWIQT

SEQ ID NO: 15 - DNA, 702 bp, Artificial Sequence
  Human TK1 mutV84DG90A
atgagctgcattaacctgcccactgtgctgcccggctcccccagcaagacccgggggcagatccaggtgattctcgggccgatgttctcaggaaaaagcacagagttgatgagacgcgtccgtcgcttccagattgctcagtacaagtgcctggtgatcaagtatgccaaagacactcgctacagcagcagcttctgcacacatgaccggaacaccatggaggcgctgcccgcctgcctgctccgagacgacgcccaggaggccctggctgtggctgtcataggcatcgacgaggggcagtttttccctgacatcgtggagttctgcgaggccatggccaacgccgggaagaccgtaattgtggctgcactggatgggaccttccagaggaagccatttggggccatcctgaacctggtgccgctggccgagagcgtggtgaagctgacggcggtgtgcatggagtgcttccgggaagccgcctataccaagaggctcggcacagagaaggaggtcgaggtgattgggggagcagacaagtaccactccgtgtgtcggctctgctacttcaagaaggcctcaggccagcctgccgggccggacaacaaagagaactgcccagtgccaggaaagccaggggaagccgtggctgccaggaagctctttgccccacagcagattctgcaatggatccagacatga

SEQ ID NO: 16 - protein, 233 aa, Artificial Sequence
  Human TK1 mutV84DG90A
MSCINLPTVLPGSPSKTRGQIQVILGPMFSGKSTELMRRVRRFQIAQYKCLVIKYAKDTRYSSSFCTHDRNTMEALPACLLRDDAQEALAVAVIGIDEGQFFPDIVEFCEAMANAGKTVIVAALDGTFQRKPFGAILNLVPLAESVVKLTAVCMECFREAAYTKRLGTEKEVEVIGGADKYHSVCRLCYFKKASGQPAGPDNKENCPVPGRPGEAVAARKLFAPQQILQWIQT

SEQ ID NO: 17 - DNA, 702 bp, Artificial Sequence
  Human TK1 mutA75GA84EG90A
atgagctgcattaacctgcccactgtgctgcccggctcccccagcaagacccgggggcagatccaggtgattctcgggccgatgttctcaggaaaaagcacagagttgatgagacgcgtccgtcgcttccagattgctcagtacaagtgcctggtgatcaagtatgccaaagacactcgctacagcagcagcttctgcacacatgaccggaacaccatggagggcctgcccgcctgcctgctccgagacgaggcccaggaggccctggctgtggctgtcataggcatcgacgaggggcagtttttccctgacatcgtggagttctgcgaggccatggccaacgccgggaagaccgtaattgtggctgcactggatgggaccttccagaggaagccatttggggccatcctgaacctggtgccgctggccgagagcgtggtgaagctgacggcggtgtgcatggagtgcttccgggaagccgcctataccaagaggctcggcacagagaaggaggtcgaggtgattgggggagcagacaagtaccactccgtgtgtcggctctgctacttcaagaaggcctcaggccagcctgccgggccggacaacaaagagaactgcccagtgccaggaaagccaggggaagccgtggctgccaggaagctctttgccccacagcagattctgcaatggatccagacatga

SEQ ID NO: 18 - protein, 233 aa, Artificial Sequence
  Human TK1 mutA75GV84EG90A
MSCINLPTVLPGSPSKTRGQIQVILGPMFSGKSTELMRRVRRFQIAQYKCLVIKYAKDTRYSSSFCTHDRNTMEGLPACLLRDEAQEALAVAVIGIDEGQFFPDIVEFCEAMANAGKTVIVAALDGTFQRKPFGAILNLVPLAESVVKLTAVCMECFREAAYTKRLGTEKEVEVIGGADKYHSVCRLCYFKKASGQPAGPDNKENCPVPGRPGEAVAARKLFAPQQILQWIQT

SEQ ID NO: 19 - DNA, 702 bp, Artificial Sequence
  Human TK1 mutG90D
atgagctgcattaacctgcccactgtgctgcccggctcccccagcaagacccgggggcagatccaggtgattctcgggccgatgttctcaggaaaaagcacagagttgatgagacgcgtccgtcgcttccagattgctcagtacaagtgcctggtgatcaagtatgccaaagacactcgctacagcagcagcttctgcacacatgaccggaacaccatggaggcgctgcccgcctgcctgctccgagacgtggcccaggaggccctggacgtggctgtcataggcatcgacgaggggcagtttttccctgacatcgtggagttctgcgaggccatggccaacgccgggaagaccgtaattgtggctgcactggatgggaccttccagaggaagccatttggggccatcctgaacctggtgccgctggccgagagcgtggtgaagctgacggcggtgtgcatggagtgcttccgggaagccgcctataccaagaggctcggcacagagaaggaggtcgaggtgattgggggagcagacaagtaccactccgtgtgtcggctctgctacttcaagaaggcctcaggccagcctgccgggccggacaacaaagagaactgcccagtgccaggaaagccaggggaagccgtggctgccaggaagctctttgccccacagcagattctgcaatggatccagacatga

SEQ ID NO: 20 - protein, 233 aa, Artificial Sequence
  Human TK1 mutG90D
MSCINLPTVLPGSPSKTRGQIQVILGPMFSGKSTELMRRVRRFQIAQYKCLVIKYAKDTRYSSSFCTHDRNTMEALPACLLRDVAQEALDVAVIGIDEGQFFPDIVEFCEAMANAGKTVIVAALDGTFQRKPFGAILNLVPLAESVVKLTAVCMECFREAAYTKRLGTEKEVEVIGGADKYHSVCRLCYFKKASGQPAGPDNKENCPVPGRPGEAVAARKLFAPQQILQWIQT

SEQ ID NO: 21 - DNA, 702 bp, Artificial Sequence
  Human TK1 mutG90V
atgagctgcattaacctgcccactgtgctgcccggctcccccagcaagacccgggggcagatccaggtgattctcgggccgatgttctcaggaaaaagcacagagttgatgagacgcgtccgtcgcttccagattgctcagtacaagtgcctggtgatcaagtatgccaaagacactcgctacagcagcagcttctgcacacatgaccggaacaccatggaggcgctgcccgcctgcctgctccgagacgtggcccaggaggccctggtggtggctgtcataggcatcgacgaggggcagtttttccctgacatcgtggagttctgcgaggccatggccaacgccgggaagaccgtaattgtggctgcactggatgggaccttccagaggaagccatttggggccatcctgaacctggtgccgctggccgagagcgtggtgaagctgacggcggtgtgcatggagtgcttccgggaagccgcctataccaagaggctcggcacagagaaggaggtcgaggtgattgggggagcagacaagtaccactccgtgtgtcggctctgctacttcaagaaggcctcaggccagcctgccgggccggacaacaaagagaactgcccagtgccaggaaagccaggggaagccgtggctgccaggaagctctttgccccacagcagattctgcaatggatccagacatga

SEQ ID NO: 22 - protein, 233 aa, Artificial Sequence
  Human TK1 mutG90V
MSCINLPTVLPGSPSKTRGQIQVILGPMFSGKSTELMRRVRRFQIAQYKCLVIKYAKDTRYSSSFCTHDRNTMEALPACLLRDVAQEALVVAVIGIDEGQFFPDIVEFCEAMANAGKTVIVAALDGTFQRKPFGAILNLVPLAESVVKLTAVCMECFREAAYTKRLGTEKEVEVIGGADKYHSVCRLCYFKKASGQPAGPDNKENCPVPGRPGEAVAARKLFAPQQILQWIQT

SEQ ID NO: 23 - DNA, 702 bp, Artificial Sequence
  Human TK1 mutA75QV84E
atgagctgcattaacctgcccactgtgctgcccggctcccccagcaagacccgggggcagatccaggtgattctcgggccgatgttctcaggaaaaagcacagagttgatgagacgcgtccgtcgcttccagattgctcagtacaagtgcctggtgatcaagtatgccaaagacactcgctacagcagcagcttctgcacacatgaccggaacaccatggagcagctgcccgcctgcctgctccgagacgaggcccaggaggccctgggcgtggctgtcataggcatcgacgaggggcagtttttccctgacatcgtggagttctgcgaggccatggccaacgccgggaagaccgtaattgtggctgcactggatgggaccttccagaggaagccatttggggccatcctgaacctggtgccgctggccgagagcgtggtgaagctgacggcggtgtgcatggagtgcttccgggaagccgcctataccaagaggctcggcacagagaaggaggtcgaggtgattgggggagcagacaagtaccactccgtgtgtcggctctgctacttcaagaaggcctcaggccagcctgccgggccggacaacaaagagaactgcccagtgccaggaaagccaggggaagccgtggctgccaggaagctctttgccccacagcagattctgcaatggatccagacatga

SEQ ID NO: 24 - protein, 233 aa, Artificial Sequence
  Human TK1 mutA75QV84E
MSCINLPTVLPGSPSKTRGQIQVILGPMFSGKSTELMRRVRRFQIAQYKCLVIKYAKDTRYSSSFCTHDRNTMEQLPACLLRDEAQEALGVAVIGIDEGQFFPDIVEFCEAMANAGKTVIVAALDGTFQRKPFGAILNLVPLAESVVKLTAVCMECFREAAYTKRLGTEKEVEVIGGADKYHSVCRLCYFKKASGQPAGPDNKENCPVPGRPGEAVAARKLFAPQQILQWIQT

SEQ ID NO: 25 - DNA, 702 bp, Artificial Sequence
  Human TK1 mutL8081F
atgagctgcattaacctgcccactgtgctgcccggctcccccagcaagacccgggggcagatccaggtgattctcgggccgatgttctcaggaaaaagcacagagttgatgagacgcgtccgtcgcttccagattgctcagtacaagtgcctggtgatcaagtatgccaaagacactcgctacagcagcagcttctgcacacatgaccggaacaccatggaggcactgcccgcctgcttcttccgagacgtggcccaggaggccctgggcgtggctgtcataggcatcgacgaggggcagtttttccctgacatcgtggagttctgcgaggccatggccaacgccgggaagaccgtaattgtggctgcactggatgggaccttccagaggaagccatttggggccatcctgaacctggtgccgctggccgagagcgtggtgaagctgacggcggtgtgcatggagtgcttccgggaagccgcctataccaagaggctcggcacagagaaggaggtcgaggtgattgggggagcagacaagtaccactccgtgtgtcggctctgctacttcaagaaggcctcaggccagcctgccgggccggacaacaaagagaactgcccagtgccaggaaagccaggggaagccgtggctgccaggaagctctttgccccacagcagattctgcaatggatccagacatga

SEQ ID NO: 26 - protein, 233 aa, Artificial Sequence
  Human TK1 mutL8081F
MSCINLPTVLPGSPSKTRGQIQVILGPMFSGKSTELMRRVRRFQIAQYKCLVIKYAKDTRYSSSFCTHDRNTMEALPACFFRDVAQEALGVAVIGIDEGQFFPDIVEFCEAMANAGKTVIVAALDGTFQRKPFGAILNLVPLAESVVKLTAVCMECFREAAYTKRLGTEKEVEVIGGADKYHSVCRLCYFKKASGQPAGPDNKENCPVPGRPGEAVAARKLFAPQQILQWIQT

SEQ ID NO: 27 - DNA, 705 bp, Homo sapiens
  Human TK1 wt NM_003258 (ORF)
atgagctgcattaacctgcccactgtgctgcctggctcccccagcaagacccgggggcagatccaggtgattctcgggccgatgttctcaggaaaaagcacagagttgatgagacgcgtccgtcgcttccagattgctcagtacaagtgcctggtgatcaagtatgccaaagacactcgctacagcagcagcttctgcacacatgaccggaacaccatggaggcactgcccgcctgcctgctccgagacgtggcccaggaggccctgggcgtggctgtcataggcatcgacgaggggcagtttttccctgacatcgtggagttctgcgaggccatggccaacgccgggaagaccgtaattgtggctgcactggatgggaccttccagaggaagccatttggggccatcctgaacctggtgccgctggccgagagcgtggtgaagctgacggcggtgtgcatggagtgcttccgggaagccgcctataccaagaggctcggcacagagaaggaggtcgaggtgattgggggagcagacaagtaccactccgtgtgtcggctctgctacttcaagaaggcctcaggccagcctgccgggccggacaacaaagagaactgcccagtgccaggaaagccaggggaagccgtggctgccaggaagctctttgccccacagcagattctgcaatgcagccctgccaactga

SEQ ID NO: 28 - protein, 234 aa, Homo sapiens
  Human TK1 wt NM_003258
MSCINLPTVLPGSPSKTRGQIQVILGPMFSGKSTELMRRVRRFQIAQYKCLVIKYAKDTRYSSSFCTHDRNTMEALPACLLRDVAQEALGVAVIGIDEGQFFPDIVEFCEAMANAGKTVIVAALDGTFQRKPFGAILNLVPLAESVVKLTAVCMECFREAAYTKRLGTEKEVEVIGGADKYHSVCRLCYFKKASGQPAGPDNKENCPVPGKPGEAVAARKLFAPQQILQCSPAN

SEQ ID NO: 29 - DNA, 1616 bp, Homo sapiens
gcgcacgtcccggattcctcccacgagggggcgggctgcggccaaatctcccgccaggtcagcggccgggcgctgattggccccatggcggcggggccggctcgtgattggccagcacgccgtggtttaaagcggtcggcgcgggaaccaggggcttactgcgggacggccttggagagtactcgggttcgtgaacttcccggaggcgcaatgagctgcattaacctgcccactgtgctgcctggctcccccagcaagacccgggggcagatccaggtgattctcgggccgatgttctcaggaaaaagcacagagttgatgagacgcgtccgtcgcttccagattgctcagtacaagtgcctggtgatcaagtatgccaaagacactcgctacagcagcagcttctgcacacatgaccggaacaccatggaggcactgcccgcctgcctgctccgagacgtggcccaggaggccctgggcgtggctgtcataggcatcgacgaggggcagtttttccctgacatcgtggagttctgcgaggccatggccaacgccgggaagaccgtaattgtggctgcactggatgggaccttccagaggaagccatttggggccatcctgaacctggtgccgctggccgagagcgtggtgaagctgacggcggtgtgcatggagtgcttccgggaagccgcctataccaagaggctcggcacagagaaggaggtcgaggtgattgggggagcagacaagtaccactccgtgtgtcggctctgctacttcaagaaggcctcaggccagcctgccgggccggacaacaaagagaactgcccagtgccaggaaagccaggggaagccgtggctgccaggaagctctttgccccacagcagattctgcaatgcagccctgccaactgagggacctgcgagggccgcccgctcccttcctgccactgccgcctactggacgctgccctgcatgctgcccagccactccaggaggaagtcgggaggcgtggagggtgaccacaccttggccttctgggaactctcctttgtgtggctgccccacctgccgcatgctccctcctctcctacccactggtctgcttaaagcttccctctcagctgctgggacgatcgcccaggctggagctggccccgcttggtggcctgggatctggcacactccctctccttggggtgagggacagagccccacgctgttgacatcagcctgcttcttcccctctgcggctttcactgctgagtttctgttctccctgggaagcctgtgccagcacctttgagccttggcccacactgaggcttaggcctctctgcctgggatgggctcccaccctcccctgaggatggcctggattcacgccctcttgtttccttttgggctcaaagcccttcctacctctggtgatggtttccacaggaacaacagcatctttcaccaagatgggtggcaccaaccttgctgggacttggatcccaggggcttatctcttcaagtgtggagagggcagggtccacgcctctgctgtagcttatgaaattaactaattgaaaattcactggttggtggaaaaaaaaaaaaaaaaaaaaaa

SEQ ID NO: 30 - DNA, 702 bp, Artificial Sequence
  Human TK1 mut G90A
atgagctgcattaacctgcccactgtgctgcccggctcccccagcaagacccgggggcagatccaggtgattctcgggccgatgttctcaggaaaaagcacagagttgatgagacgcgtccgtcgcttccagattgctcagtacaagtgcctggtgatcaagtatgccaaagacactcgctacagcagcagcttctgcacacatgaccggaacaccatggaggcgctgcccgcctgcctgctccgagacgtggcccaggaggccctggctgtggctgtcataggcatcgacgaggggcagtttttccctgacatcgtggagttctgcgaggccatggccaacgccgggaagaccgtaattgtggctgcactggatgggaccttccagaggaagccatttggggccatcctgaacctggtgccgctggccgagagcgtggtgaagctgacggcggtgtgcatggagtgcttccgggaagccgcctataccaagaggctcggcacagagaaggaggtcgaggtgattgggggagcagacaagtaccactccgtgtgtcggctctgctacttcaagaaggcctcaggccagcctgccgggccggacaacaaagagaactgcccagtgccaggaaggccaggggaagccgtggctgccaggaagctctttgccccacagcagattctgcaatggatccagacatga

SEQ ID NO: 31 - protein, 233 aa, Artificial Sequence
  mutant human thymidine kinase
MSCINLPTVLPGSPSKTRGQIQVILGPMFSGKSTELMRRVRRFQIAQYKCLVIKYAKDTRYSSSFCTHDRNTMEALPACLLRDVAQEALAVAVIGIDEGQFFPDIVEFCEAMANAGKTVIVAALDGTFQRKPFGAILNLVPLAESVVKLTAVCMECFREAAYTKRLGTEKEVEVIGGADKYHSVCRLCYFKKASGQPAGPDNKENCPVPGKPGEAVAARKLFAPQQILQWIQT

SEQ ID NO: 32 - protein, 25 aa, Artificial Sequence
  human wtTk1
NTMEALPACLLRDVAQEALGVAVIG

SEQ ID NO: 33 - protein, 25 aa, Artificial Sequence
  human TK1 mutM73AA75DD83AG9095A
NTAEDLPACLLRAVAQEALAVAVIA

SEQ ID NO: 34 - protein, 25 aa, Artificial Sequence
  human TK1 mutM73AA75DD83AG90A
NTAEDLPACLLRAVAQEALAVAVIG

SEQ ID NO: 35 - protein, 25 aa, Artificial Sequence
  human TK1 mut V84EG90A
NTMEALPACLLRDEAQEALAVAVIG

SEQ ID NO: 36 - protein, 25 aa, Artificial Sequence
  human TK1 mutM73AA75DG9095A
NTAEDLPACLLRDVAQEALAVAVIA

SEQ ID NO: 37 - protein, 25 aa, Artificial Sequence
  human TK1 mutM73AA75DG90A
NTAEDLPACLLRDVAQEALAVAVIG

SEQ ID NO: 38 - protein, 25 aa, Artificial Sequence
  human TK1 mut G90A
NTMEALPACLLRDVAQEALAVAVIG

SEQ ID NO: 39 - protein, 25 aa, Artificial Sequence
  human TK 1 mut A75DG90E
NTMEDLPACLLRDVAQEALEVAVIG

SEQ ID NO: 40 - protein, 25 aa, Artificial Sequence
  human TK1 mut V84DG90A
NTMEALPACLLRDDAQEALAVAVIG

SEQ ID NO: 41 - protein, 25 aa, Artificial Sequence
  human TK 1 mut A75GV84EG90A
NTMEGLPACLLRDEAQEALAVAVIG

SEQ ID NO: 42 - protein, 25 aa, Artificial Sequence
  human TK1 mut G90D
NTMEALPACLLRDVAQEALDVAVIG

SEQ ID NO: 43 - protein, 25 aa, Artificial Sequence
  human TK1 mut G90V
NTMEALPACLLRDVAQEALVVAVIG

SEQ ID NO: 44 - protein, 25 aa, Artificial Sequence
  human TK 1 mut A75QV84E
NTMEQLPACLLRDEAQEALGVAVIG

SEQ ID NO: 45 - protein, 25 aa, Artificial Sequence
  human TK1 mut L8081F
NTMEALPACFFRDVAQEALGVAVIG

SEQ ID NO: 46 - DNA, 20 bp, Artificial Sequence
  superTK1 antisense (pUHDhTk1as)
attaacctgcccactgtgct

SEQ ID NO: 47 - DNA, 19 bp, Artificial Sequence
  superTK1 sense (pUHDhumTK1s)
ctgtggggcaaagagcttc

SEQ ID NO: 48 - DNA, 19 bp, Artificial Sequence
  superTK1 sense (pUHDhumTK1s)
ctgtgggccaaagagcttc

SEQ ID NO: 49 - DNA, 20 bp, Artificial Sequence
  hygromycin sense
gatgtaggagggcgtggata

SEQ ID NO: 50 - DNA, 20 bp, Artificial Sequence
  hygromycin antisense
ataggtcaggctctcgctga

SEQ ID NO: 51 - DNA, 18 bp, Artificial Sequence
  nucleotide sequence encoding CSPAN
tgcagccctgccaactga

SEQ ID NO: 52 - protein, 5 aa, Artificial Sequence
  C-terminus of wt tymidine kinase
CSPAN

SEQ ID NO: 53 - protein, 4 aa, Artificial Sequence
  C-terminus of mutant tymidine kinase
WIQT

(19) United States
(12) Patent Application Publication
(10) Pub. No.: US 2021/0085756 A1
(43) Pub. Date: Mar. 25, 2021

(54) SUPERACTIVE MUTANT THYMIDINE KINASE FOR USE IN CANCER THERAPY

(71) Applicant: MEDIZINISCHE UNIVERSITÄT WIEN, Wien (AT)

(72) Inventor: Reinhold HOFBAUER, Wien (AT)

(73) Assignee: MEDIZINISCHE UNIVERSITÄT WIEN, Wien (AT)

(21) Appl. No.: 16/633,610

(22) PCT Filed: Jul. 25, 2018
(86) PCT No.: PCT/EP2018/070203

§ 371 Date: Jan. 24, 2020

(30) Foreign Application Priority Data
Jul. 26, 2017 (EP) 17183361.9

(51) Int. Cl. A61K 38/45; C12N 9/12; C12N 15/86; A61P 35/00

(52) U.S. Cl. CPC A61K 38/45; A61P 35/00; C12N 15/86; C12N 9/1211

(57) ABSTRACT

The present invention relates to a mutant thymidine kinase, specifically a mutant human thymidine kinase 1 (Tkl1). The activity of the mutant thymidine kinase is increased compared to the activity of wildtype thymidine kinase. Provided herein are uses of the mutant thymidine kinase in therapy, such as in cancer therapy. Also methods of treating cancer comprising administering the mutant thymidine kinase are disclosed herein. The present invention relates, inter alia, to a nucleic acid for use hi treating cancer, wherein said nucleic acid comprises a nucleotide sequence, wherein said nucleotide sequence encodes a mutant thymidine kinase.

The present invention relates to a mutant thymidine kinase, specifically a mutant human thymidine kinase 1 (Tk1). The activity of the mutant thymidine kinase is increased compared to the activity of wildtype thymidine kinase. Provided herein are uses of the mutant thymidine kinase in therapy, such as in cancer therapy. Also methods of treating cancer comprising administering the mutant thymidine kinase are disclosed herein. The present invention relates, inter alia, to a nucleic acid for use in treating cancer, wherein said nucleic acid comprises a nucleotide sequence, wherein said nucleotide sequence encodes a mutant thymidine kinase.

Cancer is still a major health problem worldwide and innovative therapies are needed. Gene therapy offers promising techniques for the development of anticancer agents. A way of selectively targeting and killing tumor cells is to take advantage of tumor-colonizing microorganisms, such as viruses. These microorganisms have the advantage that they colonize and replicate in primary tumors and metastases to a much higher extent than in healthy tissues. This feature is either a consequence of rational genetic engineering or a natural trait of the microorganisms [1]. Those vectors, as the microorganisms are called, have in common that they are used to (over)express therapeutic proteins or inhibit oncogenes in tumor tissue. The main mechanisms mediating the therapeutic effect of transgene expression include interference with neoangiogenesis, blockage of cell division, promotion of apoptosis and sensitization to chemotherapy, delivery of cytotoxic genes, and activation of anticancer immune responses. Many cytotoxic genes are derived from pathogenic bacteria, e.g. genes encoding diphteria toxin, Pseudomonas exotoxin A, caspases, streptolysin or melittin [2]. Bacteriophage λ-holin protein showed satisfying results in mammary cancer cell xenografts. The effect of the λ-holin protein on eukaryotic cells was studied in vitro by viability assays and in vivo in a mouse model.

Analysis of cell viability demonstrated a highly effective cell killing effect upon tet-controlled induction of λ-holin protein synthesis. Moreover, human mammary tumour cell xenografts established in immunocompetent mice as well as murine mammary adenocarcinoma cell-derived tumours in syngeneic Balb/c mice exhibited significantly reduced growth rates when the λ-holin-encoding sequence was expressed [3]. Other cytotoxic genes are coding for endogenous human enzymes. Tumor necrosis factor (TNF) a is a cytokine with immunostimulatory and cytotoxic properties, particularly affecting the tumor vasculature and the cytotoxic activity. pDNA encoding (TNF)-α has been systemically delivered to subcutaneous tumors of mice and was expressed in tumor cells close to the feeding vessel. Locally produced TNF protein is assumed to be responsible for destruction of the tumor vasculature, resulting in the observed tumor necrosis [4]. Various experiments on several cytotoxic genes regarding their suitability in cancer gene therapy have been performed [5]. Thymidin kinases (TKs) have a key function in DNA synthesis, as they offer the only way to introduce deoxythymidine (dTh) into the cell. The basic function of the wild type enzyme consists of phosphorylating deoxythymidines to deoxythymidine monophosphate (dTMP) thus importing the nucleoside into the cell and converting it into a nucleotide. The TKs in general have therefore the main ability to control the intracellular dTMP pool, which is important for eukaryotic cells. In mammalian cells two genetically and biochemically very different isoforms of the thymidine kinase exist: the Tk1, which is present in the cytoplasm of the cells, and the Tk 2, which is located in the mitochondria [6]. In human cells the genes for the isoforms are encoded on distinct chromosomes: Tk1 on chr17 [7, 8] and Tk2 on chr16 [9]. Whereas the Tk1 activity is high in dividing and malignant cells and low in quiescent cells [10-14], the Tk2 activity is low in all of them and not dependent on the cell cycle status of the cells, it is constitutively active [10]. The Tk1, a key enzyme of the salvage pathway, has a central function for antiviral and/or cytostatic treatment. Thymidine kinase1 plays an active role in cell division. It is thus often overexpressed in cancer cells and therefore used as a tumor marker. Thus, it would be counter-intuitive to increase its expression or activity to treat cancer. In the scientific literature numerous publications can be found where the cytosolic thymidine kinase 1 is described as a useful and reliable tumor marker presenting a highly prognostic and monitoring value, since it is regulated and induced during growth and cell cycle. [13, 15-21]. This feature is a consequence of the fact that cancer cells have to induce this important gene to replenish ongoing nucleotide supply driving DNA synthesis and cell proliferation. Therefore, it would be expected that a superactive thymidine kinase 1 is more likely beneficial for tumor cell growth rather than a useful tool for tumor cell growth inhibition. Based on this knowledge, one has to expect that artificially increased Tk1 activity could dangerously enhance tumor cell growth. Indeed, up to now it has not been proposed that thymidine kinase1 (Tk1) itself should be used as an active therapeutic agent in therapy, particularly cancer therapy.

Deoxythymidine (dTh) has a long history as a cell synchronizing agent in mammalian cell culture and as a radioactive labelling tool to trace ongoing DNA synthesis [22]. Most remarkably about three decades ago it was investigated whether it might be used as a clinical agent in combination with other cytostatic molecules in order to potentiate the anticancer activity of 5′-fluoruracil (5′-FU) [23], to increase the sensitivity cancer cells to cytosine arabinoside (AraC) [24], and to multiply the anti-tumor action of cyclophosphamide [25]. In normal cells an effective arrest is mainly dependent on high-dose applications of dTh (5-20 mM dTh), which in fact is the major draw-back for the regular clinical application of dTh, since a variety of unwanted side effects already occur at these levels [22]. Due to these side-effects, dTh is normally not used in cancer therapy nowadays.

In this context it is of note that for many decades the metabolite thymidine has been used to synchronize cells in the cell cycle (double thymidine block). Thymidine once imported into the cells and converted to thymidine monophosphate is up-phosphorylated to thymidine triphosphate and then able to allosterically influence ribonucleotide reductase. The elevated TTP-levels provoke deoxycytidine starvation resulting in a halt of DNA synthesis and repair and finally even having a cytotoxic effect resulting in cell death [22, 26, 27]. In addition, thymidine also exerts an inhibitory effect on poly (ADPribose) polymerase (PARP) an important function directly involved in DNA repair [28, 29]. All these inhibitory effects together were the main arguments for several research groups to initiate clinical studies with thymidine [30-33]. Finally all these studies were discontinued because of numerous side effects exerted by the high thymidine concentrations that had to be administered. Therefore, it is common ground in the art that thymidine therapy is not applicable.

The viral thymidine kinases are completely different to the mammalian enzymes in regard to both features, structure and biochemistry. In addition they are inhibited by inhibitors that do not inhibit the mammalian counterparts [34-36]. The use of mutant herpesviridae thymidine kinase has, for example, been proposed in U.S. Pat. No. 5,877,010 or WO 2014/153258 for phosphorylating nucleoside analogues like ganciclovir or AZT, an antiretroviral drug that is commonly used to treat HIV/AIDS. The mutant herpesviridae thymidine kinases are thus proposed for converting a therapeutic agent into a more active phosphorylated form, but they are not used themselves as therapeutic agent. WO 96/36365 proposes the use of Herpes simplex virus Tk to increase sensitivity to agents like ganciclovir for treating hepatocellular carcinoma.

Thus, the technical problem underlying the present invention is the provision of means and methods for cancer therapy.

The technical problem is solved by provision of the embodiments characterized in the claims.

Accordingly, the present invention relates, inter alia, to a nucleic acid for use in treating cancer, wherein said nucleic acid comprises a nucleotide sequence, wherein said nucleotide sequence encodes a mutant thymidine kinase. In a related aspect, the present invention relates to a method of treating cancer, comprising administering a nucleic acid to a subject, wherein said nucleic acid comprises a nucleotide sequence, wherein said nucleotide sequence encodes a mutant thymidine kinase.

Herein a recombinant superactive thymidine kinase 1 (superTk1) is used, that can show a more than 9-fold increased specific activity compared to the wild type enzyme (see Example 4, table 2). The terms “mutant thymidine kinase” (more specifically “mutant (human) thymidine kinase 1” and “superactive thymidine kinase” (more specifically “superactive (human) thymidine kinase 1”) can be used interchangeably herein. The term “superactive thymidine kinase” refers to a tymidine kinase with an enzymatic activity that is at least 2-fold (e.g. at least 9-fold) increased compared to the activity of the wildtype tymidine kinase and/or that that has an increase in helicity of more than 1 (e.g. more than 1.03).

The superTk1 can serve as a virtually side effect free cytostatic/-toxic function for highly specific gene therapy of solid tumors. As shown in the appended examples, the superTk1 integrated in a plasmid-based eukaryotic expression system pUHD10.3Hyg, was stable transfected into human tumor cells (HeLa, PC-3, MFM 223) and after induction by doxycyclin (tet-promoter) expressed to high levels. Alternatively, the superTk1 was integrated into an adeno-associated virus expression system (AAV) that allows the transfection into any kind of tumor cells, e.g. solid tumors in model organisms. Both recombinant systems were used to over-express superTk1mRNA in human primary tumor cells to exceedingly high levels. This led in direct consequence to a growth arrest in early S-phase in cells carrying the superTk1 expression construct and at dTh levels 50-fold less than in normal and untransfected cells used in earlier studies (about 5 mM to 20 mM dTh were used in the prior art) [e.g. 22].

Due to the fact that much lower dTh concentrations (e.g. ≥0.1 mM dTh (or even below), e.g. of from 0.01 and/or up to 5 mM dTh) in the cultivation medium are necessary, the therapeutic side effects for the non-transfected as well as nonproliferating cells are enormously reduced in context of the present invention (gentle therapy). However, dTh caused growth arrest still is independent of growth stage, origin and type of transformation (→thymidine block).

With the herein provided and used mutant tymidine kinases, a low mean plasma level or mean serum level is needed to achieve a therapeutic effect.

For example, the mean plasma level or mean serum level of dTh herein can be of from (≥) 0.01 mM dTh and/or up to (≤) 5 mM dTh, e.g. of from (≥) 0.01 mM dTh, ≥0.02 mM dTh, ≥0.03 mM dTh, or of from (≥) 0.04 mM dTh, and/or up to (≤) 0.05 mM dTh, ≤0.06 mM dTh, ≤0.07 mM dTh, ≤0.08 mM dTh. ≤0.09 mM dTh, ≤0.1 mM dTh, ≤0.2 mM dTh, ≤0.3 mM dTh, ≤0.4 mM dTh, or up to (≤) 0.5 mM dTh, or up to (≤) 0.6 mM dTh, ≤0.7 mM dTh, ≤0.8 mM dTh, ≤0.9 mM dTh, 1.0 mM dTh, ≤1.5 mM dTh, ≤2.0 mM dTh, ≤2.5 mM dTh, ≤3.0 mM dTh, ≤3.5 mM dTh, ≤4.0 mM dTh, ≤4.5 mM dTh, or ≤5.0 mM dTh. In one aspect, for example, the mean plasma level or mean serum level of dTh herein can be of from (≥) 0.01 mM dTh and/or up to (≤5) mM dTh, preferably of from (≥) 0.01 mM dTh and/or up to (≤) 0.5 mM dTh.

In a preferred aspect, the mean plasma level or mean serum level is of from (≥) 0.01 mM dTh and/or up to (≤) 0.5 mM dTh. In a preferred aspect, the mean plasma level or mean serum level is of from (≥) 0.05 mM dTh and/or up to (≤) 0.5 mM dTh. In a preferred aspect, the mean plasma level or mean serum level is of from (≥) 0.01 mM dTh and/or up to (≤) 0.05 mM dTh. In a preferred aspect, the mean plasma level or mean serum level is of from (≥) 0.1 mM dTh and/or up to (≤) 0.5 mM dTh. Particularly for very low levels of dTh (e.g. of from about (≥) 0.01 mM dTh and/or up to about (≤) 0.05 mM dTh) mutant thymidine kinases with a high activity and/or high helicity can be used, e.g. with a helicity of ≥1.16, ≥1.18, ≥1.22, ≥1.23, ≥1.30, or ≥1.35.

Among “mean plasma level” and “mean serum level” herein the “mean serum level” is preferred. The “mean serum level” refers to the level of dTh (mM dTh) in serum (serum samples) of patients (particularly human patients) to be treated herein.

Thus, the mean plasma level or mean serum level of dTh is by far lower than that used in prior art therapy. Therefore, side-effects associated with prior art dTh therapy can be avoided. With herein provided mutants (such as the herein provided “superTk1”) an inhibitory/therapeutic effect can already be achieved at normal (i.e. physiological) plasma/serum dTh levels (e.g. between about 0.01 to 0.05 mM dTh). Thus, the administration of dTh (i.e. in addition to the mutant tymidine kinase (e.g. the “super Tk1”)) may even not be necessary to achieve a therapeutic effect.

Thus, there herein provided therapy with mutant tymidine kinases (e.g. superactive Tk1) is advantageous, because remarkably reduced thymidine concentrations can be employed and are effective.

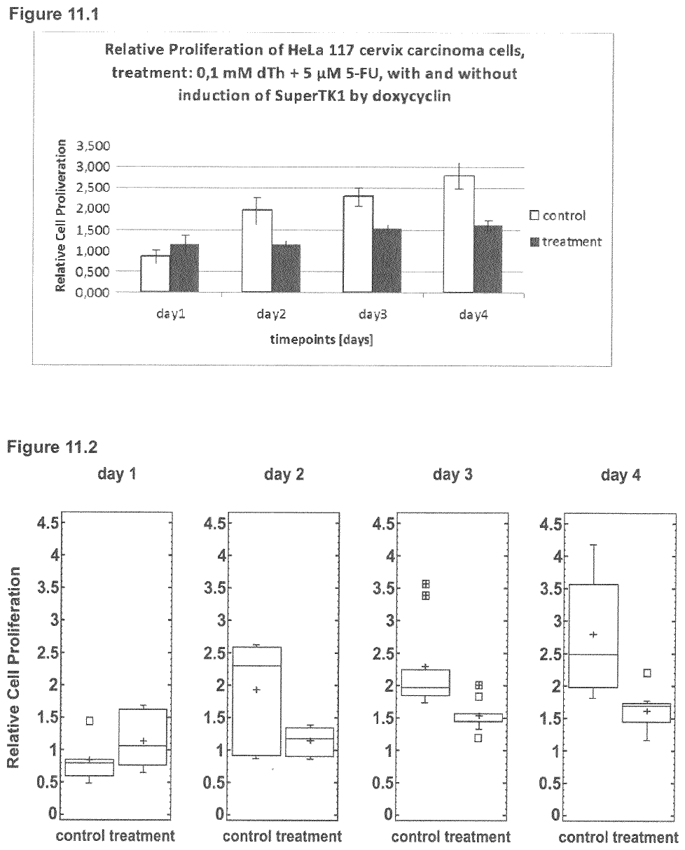

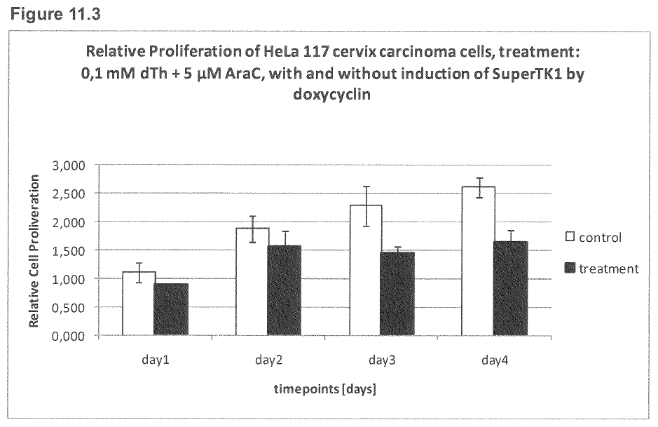

Further, a tumor cell targeted (gene) therapy as used herein avoids side-effects with non-tumorous tissue. The term “tumor cell targeted (gene) therapy” as used herein refers in particular to a therapy wherein the mutant tymidine kinase is applied to the tumor by local administration of the tymidine kinase (e.g. via vectors comprising a nucleic acid encoding the mutant tymidine kinase, like AAV vectors). For example, such vectors (like AAV vectors) are introduced into the tumor d.g. via infection of the tumor cells. The infected tumor cells produce intratumourly the mutant tymidine kinase. However, other administration routes are envisaged, e.g. the use of vectors that specifically target/infect a tumour. As an extension of the inhibition regimen synergistic effects of the nucleotide analogues 5′-fluorouracil (5-FU) and cytosine arabinoside (Cytarabine or AraC) in addition to dTh were investigated and their efficacy down to very low 1 μM serum concentrations of 5-FU or AraC was measured. In combination with 0.1 mM dTh both cytostatics still were inhibitory at 5 μM (see FIGS. 11.1 and 11.3). Experiments were performed with pUHD10.3Hyg plasmid as well as with the AAV expression system, employing the established human cell culture cell line HeLa (cervix carcinoma), followed by human primary cancer cell lines (PC-3; prostate carcinoma) and MFM223 (ductal mammary carcinoma). The experiments convincingly demonstrated cytostatic inhibition in these cancer cell lines. The AAV-driven superTk1 can be used in human tumor models with both primary cancer cells PC-3 and MFM 223 as xenografts in SCID-mice to confirm therapeutic efficacy.

As illustrated in the appended examples, the superTk1 gene to be used herein was combined with a tetracycline-controlled promoter and cloned on the pUHD10.3Hygr vector plasmid. Stable transfectants of several human tumor cell lines were produced and in vitro viability assays of HeLa cells were performed. Proof of concept was given by doxycyclin induced inhibition of proliferation rate in pUHDsuperTk1 transfected HeLa cells from day 1 on and a reduction of the cell population of more than 50% after 4 days. Inserted into a proper expression system the superTk1 is a very promising artificial gene to generate enhanced thymidine monophosphate levels in mammalian cells, leading to early S-phase cell cycle arrest (cytostatic effect) and subsequent cell death (cytotoxic effect). Thus, a “super active” thymidine kinase as provided and to be used herein can act as a cytostatic agent by inducing an early S-phase cell cycle arrest and is thus shown to be an effective therapeutic agent in cancer therapy.

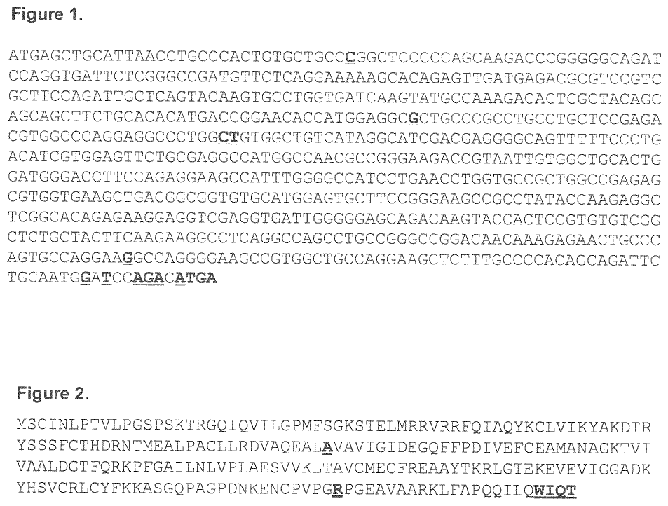

A region aa 76 to 100 of the human Tk1 (called the “thumb region”) is very sensitive for influencing the specific activity. By artificially introducing a point mutation at amino acid 90 in this domain A90G (see FIGS. 1. 2.) a more than 9 fold higher specific active Tk1 compared to the wild type enzyme was generated, termed herein: superTk1 (see Example 4, table 2). Additional mutants like as a double mutant V84DG90A supported these findings. In contrast a different double mutant L8081F (named “feeble” Tk1) entailed a strong decrease of specific activity [37]. These remarkable changes in enzymatic activity were provoked by in- or decreases in the degree of helicity of the a helix in the above mentioned Tk protein. Both mutations caused significant changes of the specific enzymatic activity of the recombinant enzyme, although the direct site of the active centre was not affected by the mutational analysis.

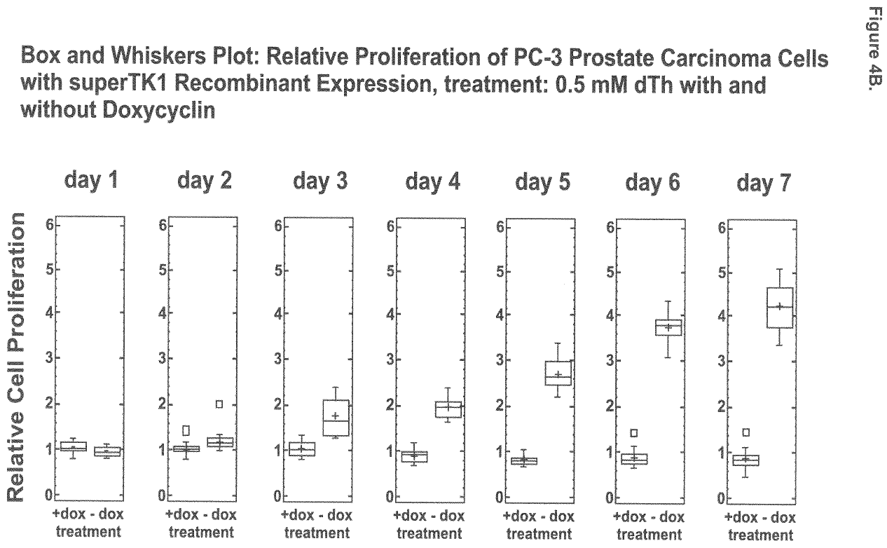

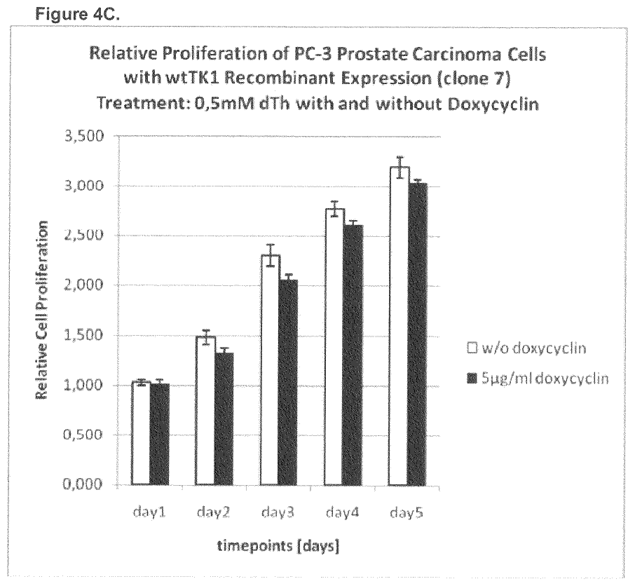

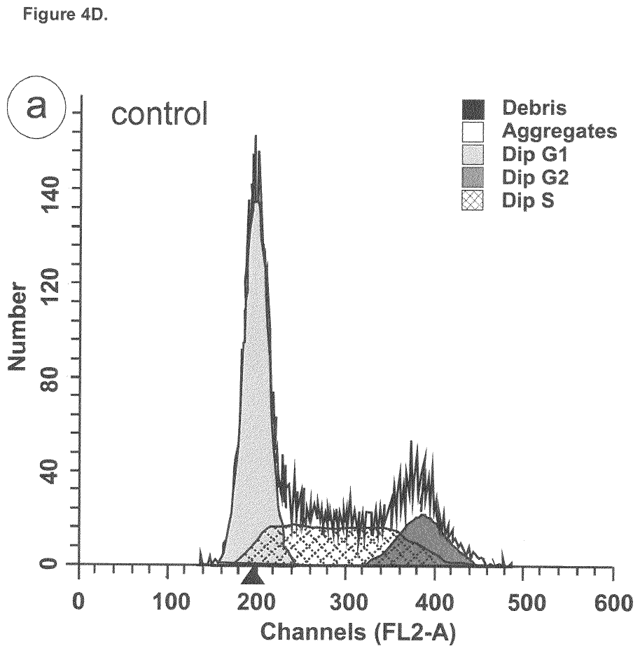

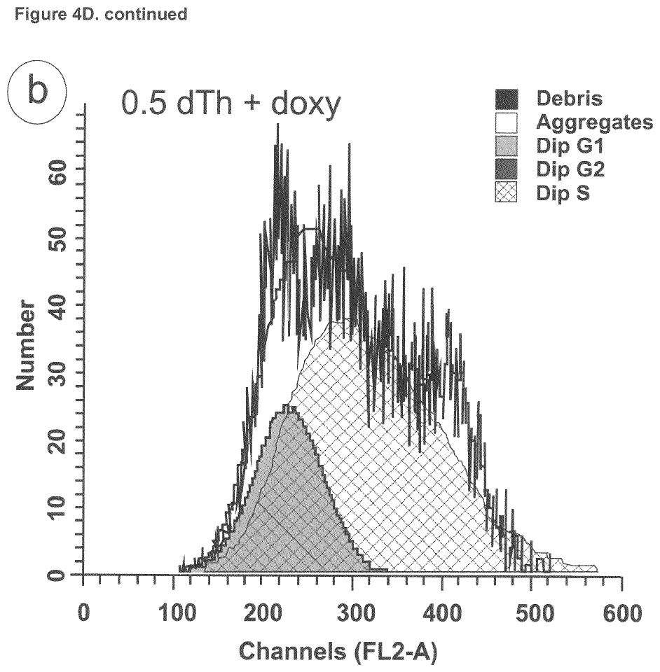

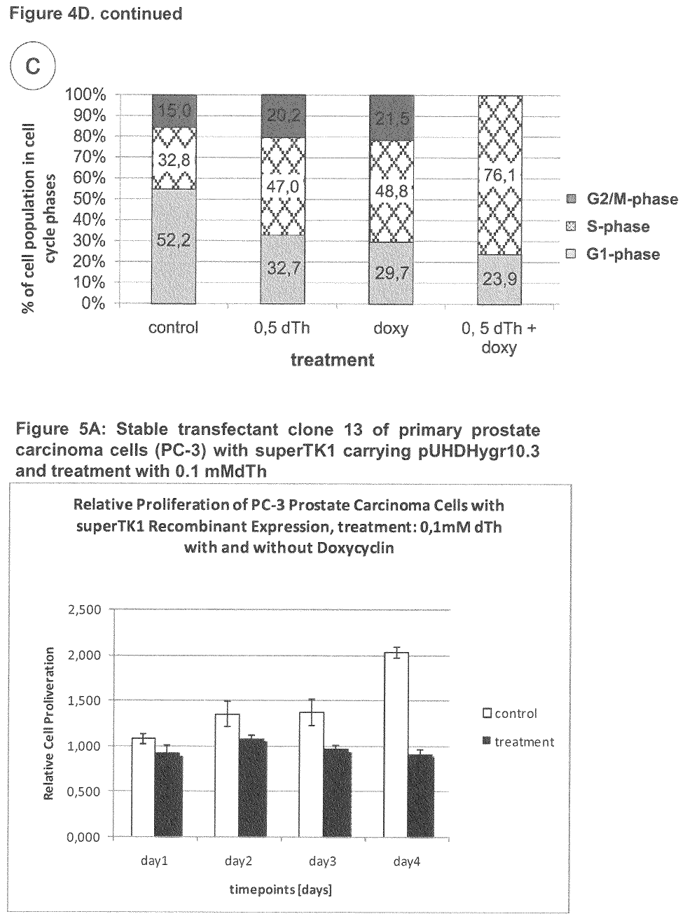

The superTk1 was used herein in a mammalian recombinant expression system to serve as a virtually side effect free cytostatic/-toxic function for highly specific gene therapy of solid tumors. The plasmid-based eukaryotic expression system pUHD10.3Hygr was ideally suited to express the superTk1. First, the constructs were stable transfected into a selection human tumor cells: HeLa (cervix carcinoma), PC-3 (primary prostate carcinoma), MFM 223 (primary ductal mammary carcinoma). Then, after induction by doxycyclin (tet-promoter), superTk1 was expressed to high levels. In all tested cases we observed a cytostatic inhibition of cellular proliferation. The growth curve analyses and statistical evaluations of dTh inhibition reaching from 0.5-0.1 mM dTh are presented in figs 4-5 and 7-9.2. Under doxycyclin induction and dTh presence in the growth media in all investigated transfectants as well as AAV carrying superTk1 infectants cell proliferation could be completely abrogated. Provoked by the enhanced Tk1-activity of the superTk1 much lower dTh concentrations (down to 0.1 mM dTh) in the cultivation medium were necessary to cause a cell growth arrest. On the contrary between 5 mM even up to 20 mM dTh would be necessary to accomplish the same job in untreated cancer cells not expressing superTk1 [38]. The growth curves presented clearly affirm these findings, in some cases even a reduction of cell number could be observed at 0.5 mM dTh, which would point to a cytotoxic effect in addition to the given cytostatic one. The cytotoxic effect can be analyzed by cell viability tests with MTT (tetrazolium salt) that is only converted by living cells with intact miotochondria and functional dehydrogenases [39]. Results indicate that a cyctotoxic effect is exerted on cells, too. However, due to the low dTh concentrations, the non-transfected as well as nonproliferating cells are not hampered at all by the administered drugs, thus minimizing side effects. The growth curves of the noninduced cells (minus doxycyclin) and the FACS analyses verify these conclusions.

That means that the superTk1 enables a very gentle almost side effect free cyctostatic gene therapy.

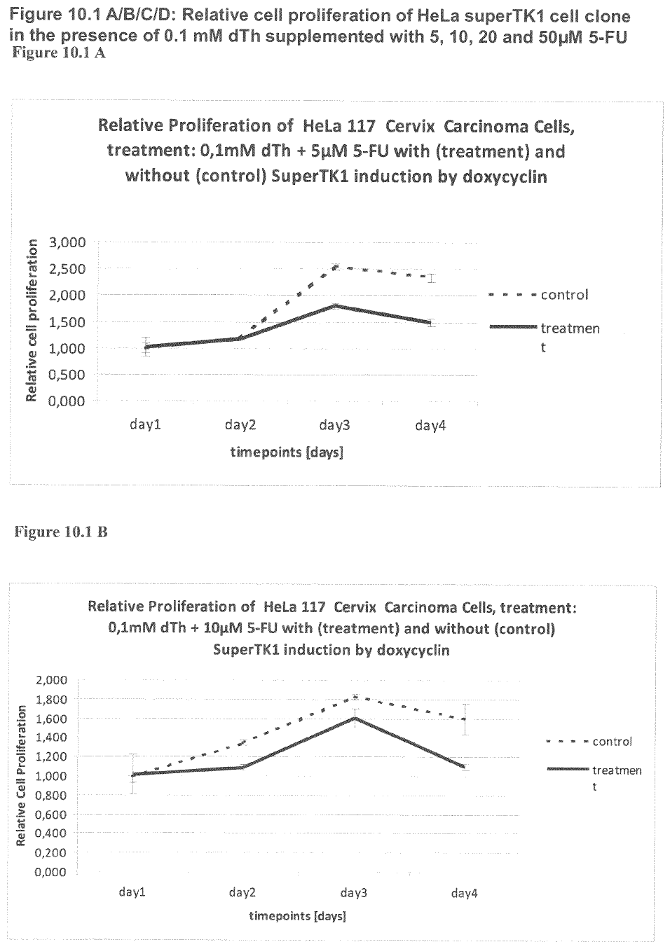

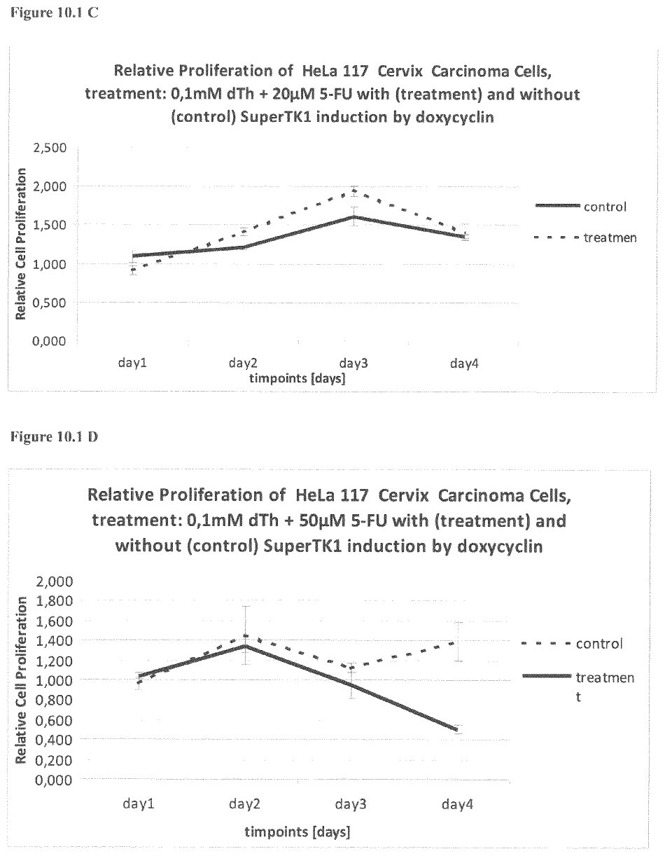

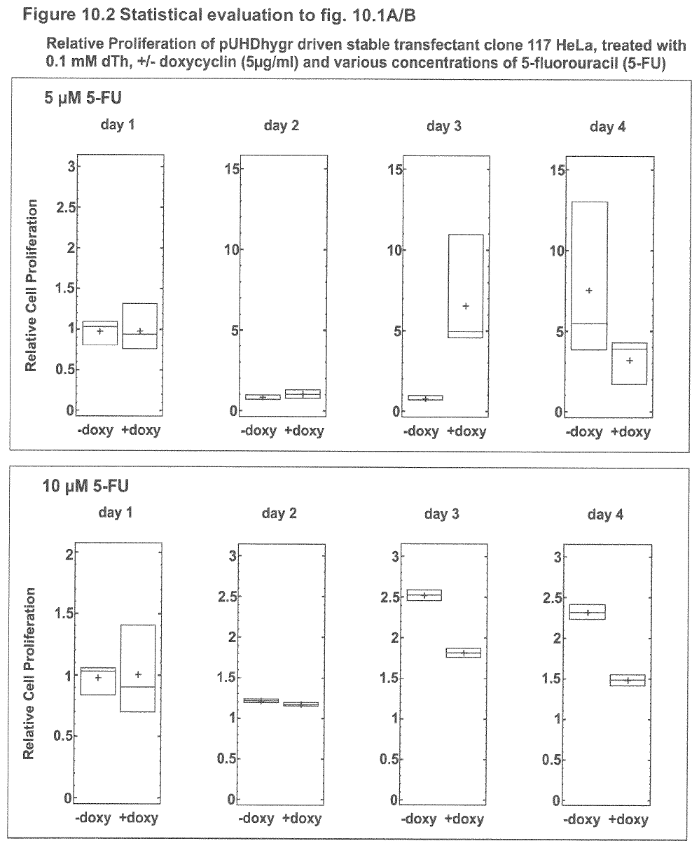

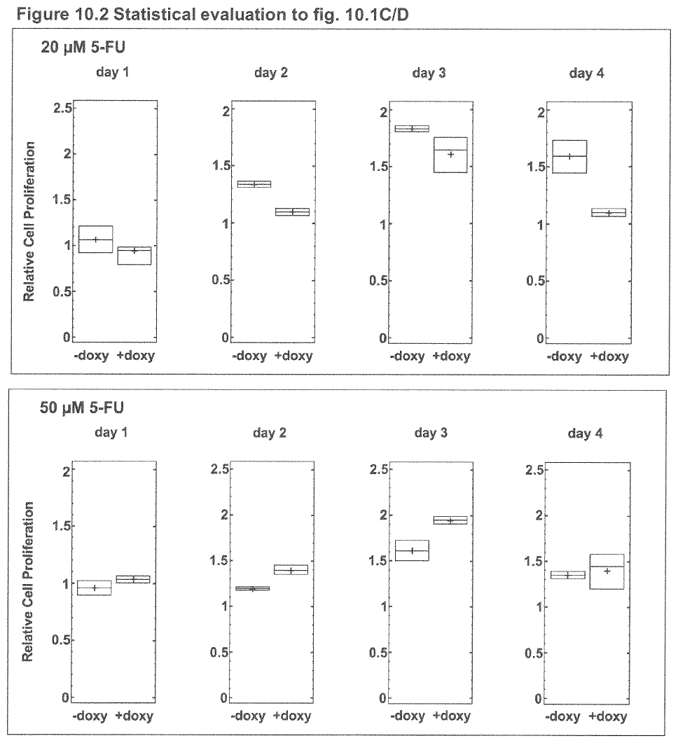

In addition to dTh the synergistic effects of the nucleotide analogues 5′-fluorouracil (5-FU) and cytosine arabinoside (Cytarabine or AraC) were investigated and their efficacy down to 1 μM serum levels was measured (see FIGS. 10.1-11.4). The striking result was that in combination with 0.1 mM dTh both cytostatic compounds were still inhibitory at very low concentrations of 5 μM in the cell culture medium.

In the experiments herein cytarabin appeared to be drug with higher cell growth arrest potential given in combination with dTh, although the differences to 5-FU were quite marginal. The most important fact was that the necessary concentration levels in the growth medium could be reduced by a factor of 50-80, compared to standard therapy regimens. At concentration levels around 1 μM AraC and 5-FU in combination with 0.1 mM dTh we reached the bottom line of growth inhibition capacity, no specific differences could be recorded anymore.

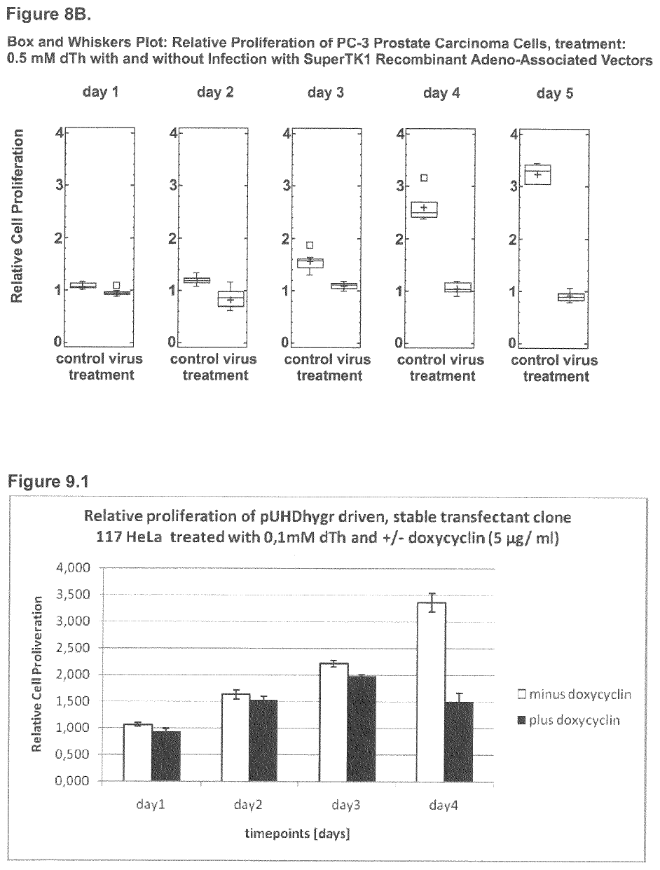

To create the basis for animal experiments the superTk1 gene was integrated into an adeno-associated virus expression system (AAV), providing the advantage to deliver by transfection the superTk1 into any kind of tumor cells, e.g. solid tumors in model organisms. It was shown that this viral infection and expression system is working as efficiently as the plasmid driven system in stable transfectants (see FIG. 8). Both recombinant systems over-express superTk1mRNA in human tumor cells to exceeding high levels that consecutively lead to a growth arrest in treated cells at much lower dTh levels (down to 0.1 mM dTh), falling below by a factor 50 underneath standard application concentrations.

This growth arrest is independent of cell type, growth stage and type of transformation (→thymidine block in early S-phase). The presented cytostatic inhibition experiments with the established human cell culture cell lines HeLa, A375, followed by human primary cancer cell lines PC-3 and MFM223 clearly proved that superTk1 can be used in cancer therapy. These results create a solid basis to confirm the therapeutic efficacy of superTk1, e.g. with the AAV-driven superTk1, in human tumor model studies with both primary cancer cells PC-3 and MFM 223 as xenografts in SCID-mice.

Without being bound by theory, it is believed that the enzymatic action of thymidine kinase itself, especially if the function is induced to high levels, is fostering the reaction dTMP giving dTTP and thus inhibiting via an end-product feedback loop other de novo and salvage pathway enzymes like ribonucleotide reductase and deoxycytidylate deaminase. The direct outcome of this influence is an inhibition of DNA synthesis at the onset of S-phase, because ribonucleotide reductase is inhibited by high dTTP levels and thus the conversion of cytidine-diphosphate to dCTP is disabled. Therefore the dTh-induced S-phase arrest is mainly caused by a depletion of the deoxycytidylatetriphosphate (dCTP) pool concentrations. These effects are finally toxic for the cells, especially if occurring for an extended period of time. These metabolic mechanisms are vital in both normal and malignant cells, and responsible for the growth inhibition at concentrations in superTk1 transformants to be used herein down to ≥1×10⁻⁴ M dTh in accordance with the present invention, e.g. in the herein provided test systems.

TKs have a key function in DNA synthesis as they offer the only way to introduce dTh into the cell. The basic function of the wild type enzyme consists of phosphorylating deoxythymidines (dTh), to deoxythymidine monophosphate (dTMP) thus importing the nucleoside into the cell and converting it into a nucleotide. The TKs in general have therefore the main ability to control the intracellular dTMP pool, which is important for eukaryotic cells. Based on theoretical studies of the amino acid sequence and the degree of helicity and hydrophobicity of specific sub-domains of the Tk1, the enzymatic action of the protein were altered to achieve enzymes with a higher enzymatic activity according to the invention. The predicted secondary structures of the Tk1 were calculated by introducing distinct changes of amino acid sequences in silico. The Tk1 mutant G90A demonstrated a remarkable increase of helicity in the “thumb” region (aa 76 to 100 of human Tk1) which caused significant changes of the specific enzymatic activity of the recombinant enzyme, although the direct site of the active center was not affected by the mutational analysis [40]. The holoenzyme was very likely stabilized by the more compact form of the protein. Tk-enzyme assays showed a 9 fold higher activity after 5 min incubation time and a 18.2 fold higher activity after 10 min incubation time compared to the wt Tk1.

In accordance with the above, further mutants of thymidine kinase are provided herein (see Example 4, Table 1 below). These mutants can be used in accordance with the herein provided teaching.

TABLE 1
summary of the human cytosolic Tk1 mutants with elevated and one with
decreased specific activity
Listed are the relevant domain aa 71-95 of the wt Tk1 (SEQ ID NO: 32) (total 234 aa CDS)
compared to the herein provided mutants (domain aa 71-95 of mutants shown in SEQ ID NOs 33 to
45 following the order of the table below), the affected and altered aa are printed in bold.
In one embodiment, the mutant thymidine kinase provided and to be used herein comprises a
domain aa 71-95 as provided herein, particularly said mutant thymidine kinase and/or said
domain aa 71-95 comprising the amino acid sequence of any one of SEQ ID NOs 33 to 44, as
shown below or in the appended sequence listing.

| Tk1aa | | |
|---|---|---|
| 71 80 90 | | |
| \| \| \| | | |
| NTMEALPACLLRDVAQEALGVAVIG human wt Tk1 | helicity increase* | |
| (in-/decrease of spec. activity if mutated) | (fold of wt Tk1) | SEQ ID |
| NTAEDLPACLLRAVAQEALAVAVIA human TK1 mutM73AA75DD83AG9095A | 1.35 | 2 |
| NTAEDLPACLLRAVAQEALAVAVIG human TK1 mutM73AA75DD83AG90A | 1.30 | 4 |
| NTMEALPACLLRDEAQEALAVAVIG human TK1 mut V84EG90A | 1.23 | 6 |
| NTAEDLPACLLRDVAQEALAVAVIA human TKI mutM73AA75DG9095A | 1.22 | 8 |
| NTAEDLPACLLRDVAQEALAVAVIG human TK1 mutM73AA75DG90A | 1.18 | 10 |
| NTMEALPACLLRDVAQEALAVAVIG human TK1 mut G90A | 1.16 | 12 |
| NTMEDLPACLLRDVAQEALEVAVIG human TK1 mut A75DG90E | 1.11 | 14 |
| NTMEALPACLLRDDAQEALAVAVIG human TK1 mut V84DG90A | 1.10 | 16 |
| NTMEGLPACLLRDEAQEALAVAVIG human TK1 mut A75GV84EG90A | 1.07 | 18 |
| NTMEALPACLLRDVAQEALDVAVIG human TK1 mut G9OD | 1.05 | 20 |
| NTMEALPACLLRDVAQEALVVAVIG human TK1 mut G90V | 1.04 | 22 |
| NTMEQLPACLLRDEAQEALGVAVIG human TK1 mut A75QV84E | 1.03 | 24 |
| NTMEALPACFFRDVAQEALGVAVIG human TK1 mut L8081F | 0.77 | 26 |

Legend to table 1:;
*the helicity calculations were performed by GOR IV secondary structure prediction method version IV provided by a network platform [41].

| | |
|---|---|
| aa73: | replacing methionine . . . nonpolar, neutral S-containing, |
| | HI=1.9 (HI = hydropathic index) |
| | with alanine . . . aliphatic, nonpolar, neutral, HI=1.8 |
| aa75: | replacing alanine . . . aliphatic, nonpolar, neutral, HI=1.8 |
| | with glycine . . . aliphatic, nonpolar, neutral, HI=−0.4 |
| | or with glutamine . . . acid/amide, polar, neutral, HI= −3.4 |
| | or with aspartic acid . . . acid/amide, acidic polar, negative, |
| | HI= −3.5 |
| aa 8081: | replacing leucine . . . aliphatic, nonpolar, neutral, HI= 3.8 |
| | with phenylalanine . . . aromatic, nonpolar, neutral, HI=2.8 |
| aa 83: | replacing aspartic acid . . . acid/amide, acidic polar, |
| | negative, HI= −3.5 |
| | with alanine . . . aliphatic, nonpolar neutral, HI= 1.8 |
| aa 84: | replacing valine . . . aliphatic, nonpolar, neutral, HI= 4.2 |
| | with glutamic acid . . . acid/amide, acidic polar, negative, |
| | HI= −3.5 |
| | or with aspartic acid . . . acid/amide, acidic polar, negative, |
| | HI= −3.5 |
| aa 90: | replacing glycine . . . aliphatic, nonpolar, neutral, HI= −0.4 |
| | with alanine . . . aliphatic, nonpolar, neutral, HI=1.8 |
| | or with valine . . . aliphatic, nonpolar, neutral, HI= 4.2 |
| | or with glutamic acid . . . acid/amide, acidic polar, negative, |
| | HI= −3.5 |
| | or with aspartic acid . . . acid/amide, acidic polar, negative, |
| | HI= −3.5 |
| aa 95: | replacing glycine . . . aliphatic, nonpolar, neutral, HI= −0.4 |
| | with alanine . . . aliphatic, nonpolar, neutral, HI=1.8 |

The strategy for generating mutations in accordance with the disclosure herein can be described as follows:

For an increase of specific activity mutate aa at positions:

| | |
|---|---|
| aa 73 | substitute methionine with an aliphatic, nonpolar and neutral |
| | aa, best option alanine |
| aa 75 | substitute alanine either with an acid/amide, acidic polar and |
| | negative aa, best option aspartic acid |
| | or with an acid/amide, polar, neutral aa like as glutamine |
| | or with an aliphatic, nonpolar and neutral aa, like as glycine |
| aa 83: | substitute aspartic acid with an aliphatic, nonpolar and |
| | neutral aa, best option alanine |
| aa 84: | substitute valine with an acid/amide, acidic polar and |
| | negative aa like as aspartic acid or glutamic acid |
| aa 90: | substitute glycine either with an aliphatic, nonpolar and |
| | neutral aa, best option alanine |
| | followed by valine |
| | or with acid/amide, acidic polar, negative aa like glutamic |
| | acid or aspartic acid |
| aa 95: | substitute glycine either with an aliphatic, nonpolar and |
| | neutral aa, best option alanine |

For a decrease of specific activity mutate aa at positions:

| | |
|---|---|
| aa 8081 | substitute both leucines with aromatic, nonpolar, neutral aa, |
| | best option phenylalanines |

TABLE 2
Correlation of the calculated protein secondary structure parameter
(degree of helicity) with the measured specific enzyme activity of
human cytosolic Tk1 mutants with elevated and one with decreased
specific activity

| humTK1- | Helicity | measured specific Tk1 | | Relative in-/decrease |
|---|---|---|---|---|
| variant | (AUC) | enzyme activity | | of helicity to wt Tk1 |
| wt | 332924 | 37 | pmol/mg × min | 1.00 |
| L8081F | 255025 | 1.4 | pmol/mg × min | 0.77 |
| G90A | 385426 | 375 | pmol/mg × min* | 1.16 |
| V84DG90A | 366809 | 393 | pmol/mg × min | 1.10 |

*data retrieved from a different experiment

Legend to table 2: Helicity scores given in the table were calculated by NPS@: Network Protein Sequence Analysis [41]; the AUC was obtained by integration of the area under the helicity curve between aa 71-95 of the Tk1 variants and equals the degree of helicity of the protein domain

The present invention relates, inter alia, to the following aspects:
1. A nucleic acid for use in treating cancer, wherein said nucleic acid comprises a nucleotide sequence, wherein said nucleotide sequence encodes a mutant human thymidine kinase.
2. A method of treating cancer, comprising administering a nucleic acid to a subject, wherein said nucleic acid comprises a nucleotide sequence, wherein said nucleotide sequence encodes a mutant human thymidine kinase.
3. The nucleic acid for use of item 1, or the method of item 2, wherein said mutant human thymidine kinase is mutant human thymidine kinase 1.
4. The nucleic acid for use of any one of items 1 or 3, or the method of item 2 or 3, wherein the activity of said mutant thymidine kinase is increased compared to the activity of wildtype thymidine kinase.
5. The nucleic acid for use of item 4, or the method of item 4, wherein said activity is specific activity.
6. The nucleic acid for use of item 4 or 5, or the method of item 4 or 5, wherein said activity of said mutant thymidine kinase is at least 9-fold increased compared to the activity of wildtype thymidine kinase.
7. The nucleic acid for use of any one of items 1 and 3 to 6, or the method of any one of items 2 to 6, wherein said mutant thymidine kinase comprises one or more amino acid substitutions at positions corresponding to positions 70 to 100 of wild-type thymidine kinase.
8. The nucleic acid for use of item 7, or the method of item 7, wherein said mutant thymidine kinase comprises one or more amino acid substitutions at positions corresponding to positions 71 to 95 of wild-type thymidine kinase.
9. The nucleic acid for use of item 8, or the method of item 8, wherein said mutant thymidine kinase comprises one or more amino acid substitutions at positions corresponding to one or more positions 73, 75, 83, 84, 90 and 95 of wild-type thymidine kinase.
10. The nucleic acid for use of item 9, or the method of item 9, wherein said mutant thymidine kinase comprises one or two amino acid substitutions at positions corresponding to positions 84 and/or 90 of wild-type thymidine kinase.
11. The nucleic acid for use of any one of items 1 and 3 to 10, or the method of any one of items 2 to 10, wherein said mutant thymidine kinase comprises one or more aliphatic, nonpolar, and neutral amino acid, one or more acid/amide, acidic polar and negatively charged amino acid and/or one or more acid/amide, polar and neutral amino acid at one or more positions corresponding to positions 70 to 100 of wild-type thymidine kinase.
12. The nucleic acid for use of any one of items 1 and 3 to 11, or the method of any one of items 2 to 11, wherein said mutant thymidine kinase comprises one or more aliphatic, nonpolar, and neutral amino acid at one or more positions corresponding to positions 73, 75, 83, 90, and 95 of wild-type thymidine kinase.
13. The nucleic acid for use of any one of items 1 and 3 to 11, or the method of any one of items 2 to 11, wherein said mutant thymidine kinase comprises one or more aliphatic, nonpolar, and neutral amino acid at one or more positions corresponding to positions 73, 83, 90, and 95 of wild-type thymidine kinase.
14. The nucleic acid for use of any one of items 1 and 3 to 11, or the method of any one of items 2 to 11, wherein said mutant thymidine kinase comprises one or more aliphatic, nonpolar, and neutral amino acid at one or more positions corresponding to positions 75 of wild-type thymidine kinase.
15. The nucleic acid for use of any one of items 1 and 3 to 11, or the method of any one of items 2 to 11, wherein said mutant thymidine kinase comprises one or more aliphatic, nonpolar, and neutral amino acid at one or more positions corresponding to position 90 of wild-type thymidine kinase.
16. The nucleic acid for use of any one of items 1 and 3 to 11, or the method of any one of items 2 to 11, wherein said mutant thymidine kinase comprises one or more acid/amide, acidic polar and negatively charged amino acid at one or more positions corresponding to positions 75, 84 and 90 of wild-type thymidine kinase.
17. The nucleic acid for use of any one of items 1 and 3 to 11, or the method of any one of items 2 to 11, wherein said mutant thymidine kinase comprises one or two acid/amide, acidic polar and negatively charged amino acid at one or two positions corresponding to positions 84 and 90 of wild-type thymidine kinase.
18. The nucleic acid for use of any one of items 1 and 3 to 11, or the method of any one of items 2 to 11, wherein said mutant thymidine kinase comprises a acid/amide, polar and neutral amino acid at a position corresponding to position 75 of wild-type thymidine kinase.
19. The nucleic acid for use of any one of items 1 and 3 to 11, or the method of any one of items 2 to 11,
   a) wherein said mutant thymidine kinase comprises an aliphatic, nonpolar, neutral amino acid at a position corresponding to position 73 of wild-type thymidine kinase;
   b) wherein said mutant thymidine kinase comprises an aliphatic, nonpolar, neutral amino acid or an acid/amide, polar, neutral amino acid or an acid/amide, acidic polar, negatively charged amino acid at a position corresponding to position 75 of wild-type thymidine kinase;
   c) wherein said mutant thymidine kinase comprises an aliphatic, nonpolar, neutral amino acid at a position corresponding to position 83 of wild-type thymidine kinase;
   d) wherein said mutant thymidine kinase comprises an acid/amide, acidic polar, negatively charged amino acid at a position corresponding to position 84 of wild-type thymidine kinase;
   e) wherein said mutant thymidine kinase comprises an aliphatic, nonpolar, neutral amino acid or an acid/amide, acidic polar, negatively charged amino acid at a position corresponding to position 90 of wild-type thymidine kinase; and/or
   f) wherein said mutant thymidine kinase comprises an aliphatic, nonpolar, neutral amino acid at a position corresponding to position 95 of wild-type thymidine kinase.
20. The nucleic acid for use of any one of items 1 and 3 to 19, or the method of any one of items 2 to 19, wherein said mutant thymidine kinase comprises
   a) aliphatic, nonpolar, and neutral amino acids at positions corresponding to positions 73, 83, 90 and 95 of wild-type thymidine kinase and an acid/amide, acidic polar and negatively charged amino acid at a position corresponding to position 75 of wild-type thymidine kinase;
   b) aliphatic, nonpolar, and neutral amino acids at positions corresponding to positions 73, 83, and 90 of wild-type thymidine kinase and an acid/amide, acidic polar and negatively charged amino acid at a position corresponding to position 75 of wild-type thymidine kinase;
   c) an acid/amide, acidic polar and negatively charged amino acid at a position corresponding to position 84 of wild-type thymidine kinase and an aliphatic, nonpolar, and neutral amino acid at a position corresponding to position 90 of wild-type thymidine kinase;
   d) aliphatic, nonpolar, and neutral amino acids at positions corresponding to positions 73, 90 and 95 of wild-type thymidine kinase and an acid/amide, acidic polar and negatively charged amino acid at a position corresponding to position 75 of wild-type thymidine kinase;
   e) aliphatic, nonpolar, and neutral amino acids at positions corresponding to positions 73 and 90 of wild-type thymidine kinase and an acid/amide, acidic polar and negatively charged amino acid at a position corresponding to position 75 of wild-type thymidine kinase;
   f) an aliphatic, nonpolar, and neutral amino acid at a position corresponding to position 90 of wild-type thymidine kinase;
   g) acid/amide, acidic polar and negatively charged amino acids at positions corresponding to positions 75 and 90 of wild-type thymidine kinase;
   h) an acid/amide, acidic polar and negatively charged amino acid at a position corresponding to position 84 of wild-type thymidine kinase and an aliphatic, nonpolar, and neutral amino acid at position corresponding to position 90 of wild-type thymidine kinase;
   i) aliphatic, nonpolar, and neutral amino acids at positions corresponding to positions 75 and 90 of wild-type thymidine kinase and an acid/amide, acidic polar and negatively charged amino acid at a position corresponding to position 84 of wild-type thymidine kinase;
   j) an acid/amide, acidic polar and negatively charged amino acid at a position corresponding to position 90 of wild-type thymidine kinase;
   k) an aliphatic, nonpolar, and neutral amino acid at a position corresponding to position 90 of wild-type thymidine kinase; or
   l) an acid/amide, polar and neutral amino acid at a position corresponding to position 75 of wild-type thymidine kinase and an acid/amide, acidic polar and negatively charged amino acid at a position corresponding to position 84 of wild-type thymidine kinase.
21. The nucleic acid for use of any one of items 1 and 3 to 19, or the method of any one of items 2 to 19, wherein said mutant thymidine kinase comprises
   a) an aliphatic, nonpolar, and neutral amino acid at a position corresponding to position 90 of wild-type thymidine kinase; or
   b) an acid/amide, acidic polar and negatively charged amino acid at a position corresponding to position 84 of wild-type thymidine kinase and an aliphatic, nonpolar, and neutral amino acid at a position corresponding to position 90 of wild-type thymidine kinase.
22. The nucleic acid for use of any one of items 11 to 15 and 19 to 21, or the method of any one of items 11 to 15 and 19 to 21, wherein said aliphatic, nonpolar, and neutral amino acid is one or more of alanine, glycine, valine, isoleucine, leucine and methionine.
23. The nucleic acid for use of any one of items 11 to 13, 19a), 19c), 19e) and 19f), or the method of any one of items 11 to 13, 19a), 19c), 19e) and 19f), wherein said aliphatic, nonpolar, and neutral amino acid is alanine.
24. The nucleic acid for use of any one of items 11, 12, 14 and 19b), or the method of any one of items 11, 12, 14 and 19b), wherein said aliphatic, nonpolar, and neutral amino acid is glycine.
25. The nucleic acid for use of any one of items 11, 12, 15 and 19e), or the method of any one of items 11, 12, 15 and 19e), wherein said aliphatic, nonpolar, and neutral amino acid is valine.
26. The nucleic acid for use of any one of items 11, 16, 17, and 19 to 21, or the method of any one of items 11, 16, 17, and 19 to 21, wherein said acid/amide, acidic polar and negatively charged amino acid is one or more of aspartic acid and glutamic acid.

27. The nucleic acid for use of any one of items 11, 16, 19b), 19d), 19e), 20a), 20b), 20d), 20e), 20g), 20h) and 20j), or the method of any one of items 11, 16, 19b), 19d), 19e), 20a), 20b), 20d), 20e), 20g), 20h) and 20j), wherein said acid/amide, acidic polar and negatively charged amino acid is aspartic acid.
28. The nucleic acid for use of any one of items 11, 17, 19d), 19e), 20c), 20g), 20i) and 20l), or the method of any one of items 11, 17, 19d), 19e), 20c), 20g), 20i) and 20l), wherein said acid/amide, acidic polar and negatively charged amino acid is glutamic acid.
29. The nucleic acid for use of any one of items 11 and 18 to 20, or the method of any one of items 11 and 18 to 20, wherein said acid/amide, polar and neutral amino acid is one or more of glutamine, asparagine.
30. The nucleic acid for use of any one of items 11, 18, 19b), and 20l), or the method of any one of items 11, 18, 19b), and 20l), wherein said acid/amide, polar and neutral amino acid is glutamine.
31. The nucleic acid for use of any one of items 1 and 3 to 30, or the method of any one of items 2 to 30, wherein said mutant thymidine kinase comprises
   a) alanine at positions corresponding to positions 73, 83, 90 and 95 of wild-type thymidine kinase and aspartic acid at a position corresponding to position 75 of wild-type thymidine kinase;
   b) alanine at positions corresponding to positions 73, 83, and 90 of wild-type thymidine kinase and aspartic acid at a position corresponding to position 75 of wild-type thymidine kinase;
   c) glutamic acid at a position corresponding to position 84 of wild-type thymidine kinase and alanine at a position corresponding to position 90 of wild-type thymidine kinase;
   d) alanine at positions corresponding to positions 73, 90 and 95 of wild-type thymidine kinase and aspartic acid at a position corresponding to position 75 of wild-type thymidine kinase;
   e) alanine at positions corresponding to positions 73, and 90 of wild-type thymidine kinase and aspartic acid at a position corresponding to position 75 of wild-type thymidine kinase;
   f) alanine at a position corresponding to position 90 of wild-type thymidine kinase; g) aspartic acid at a position corresponding to position 75 of wild-type thymidine kinase and glutamic acid at a position corresponding to position 90 of wild-type thymidine kinase;
   h) aspartic acid at a position corresponding to position 84 of wild-type thymidine kinase and alanine at position corresponding to position 90 of wild-type thymidine kinase;
   i) glycine at a position corresponding to position 75 of wild-type thymidine kinase, glutamic acid at a position corresponding to position 84 of wild-type thymidine kinase and alanine at a position corresponding to position 90 of wild-type thymidine kinase;
   j) aspartic acid at a position corresponding to position 90 of wild-type thymidine kinase;
   k) valine at a position corresponding to position 90 of wild-type thymidine kinase; or
   l) glutamine at a position corresponding to position 75 of wild-type thymidine kinase and glutamic acid at a position corresponding to position 84 of wild-type thymidine kinase.
32. The nucleic acid for use of any one of items 1 and 3 to 31, or the method of any one of items 2 to 31, wherein said mutant thymidine kinase comprises
   a) alanine at a position corresponding to position 90 of wild-type thymidine kinase; or
   b) aspartic acid amino acid at a position corresponding to position 84 of wild-type thymidine kinase and an alanine at a position corresponding to position 90 of wild-type thymidine kinase.
33. The nucleic acid for use of any one of items 1 and 3 to 32, or the method of any one of items 2 to 32, wherein said wild-type thymidine kinase is a wild-type human thymidine kinase, preferably a wild-type human thymidine kinase 1.
34. The nucleic acid for use of item 33, or the method of item 33, wherein said human wild-type thymidine kinase has an amino acid sequence shown in SEQ ID NO. 28.
35. The nucleic acid for use of any one of items 1 and 3 to 34, or the method of any one of items 2 to 34, wherein said nucleic acid is selected from the group consisting of:
   a) a nucleic acid comprising a nucleotide sequence, wherein said nucleotide sequence encodes a mutant thymidine kinase, wherein said mutant thymidine kinase comprises an amino acid sequence as depicted in any one of SEQ ID NOs: 2, 4, 6, 8, 10, 12, 14, 16, 18, 20, 22 and 24;
   b) a nucleic acid comprising a nucleotide sequence, wherein said nucleotide sequence encodes a mutant thymidine kinase, and wherein said nucleotide sequence is depicted in SEQ ID NO: 1, 3, 5, 7, 9, 11, 13, 15, 17, 19, 21 and 23;
   c) a nucleic acid hybridizing under stringent conditions to the complementary strand of the nucleic acid as defined in (a) or (b) and wherein said nucleic acid comprises a nucleotide sequence wherein said nucleotide sequence encodes a mutant thymidine kinase;
   d) a nucleic acid comprising a nucleotide sequence with at least 70% identity to the nucleotide sequence of the nucleic acids of any one of (a) to (c) and wherein said nucleotide sequence encodes a mutant thymidine kinase; and
   e) a nucleic acid comprising a nucleotide sequence which is degenerate as a result of the genetic code to the nucleotide sequence of a nucleic acid of any one of (a) to (d) and wherein said nucleotide sequence encodes a mutant thymidine kinase.
36. The nucleic acid for use of any one of items 1 and 3 to 34, or the method of any one of items 2 to 34, wherein said nucleic acid is selected from the group consisting of:
   a) a nucleic acid comprising a nucleotide sequence, wherein said nucleotide sequence encodes a mutant thymidine kinase, wherein said mutant thymidine kinase comprises an amino acid sequence as depicted in any one of SEQ ID NOs: 10 and 12;
   b) a nucleic acid comprising a nucleotide sequence, wherein said nucleotide sequence encodes a mutant thymidine kinase, and wherein nucleotide sequence is depicted in SEQ ID NO: 9 and 11;
   c) a nucleic acid hybridizing under stringent conditions to the complementary strand of the nucleic acid as defined in (a) or (b) and wherein said nucleic acid comprises a nucleotide sequence wherein said nucleotide sequence encodes a mutant thymidine kinase;
   d) a nucleic acid comprising a nucleotide sequence with at least 70% identity to the nucleotide sequence of the nucleic acids of any one of (a) to (c) and wherein said nucleotide sequence encodes a mutant thymidine kinase; and
   e) a nucleic acid comprising a nucleotide sequence which is degenerate as a result of the genetic code to the nucleotide sequence of a nucleic acid of any one of (a) to (d) and wherein said nucleotide sequence encodes a mutant thymidine kinase.
37. Nucleic acid as defined in any one of items 1 to 36.
38. Vector comprising the nucleic acid of item 37.
39. The vector of item 38, wherein said vector is a gene therapy vector.
40. The vector of item 38 or 39, wherein said vector is an AAV vector, adenovirus vector, or a lentivirus vector.
41. Protein encoded by the nucleic acid of item 37.
42. Composition comprising the nucleic acid of item 37, a vector of any one of items 38 to 40, or the protein of item 41.
43. The composition of item 42, wherein said composition is a pharmaceutical composition.
44. The nucleic acid of item 37, the vector of any one of items 38 to 40, the protein of item 41, or the composition of item 42 or 43, wherein said nucleic acid, said vector, said protein, or said composition is for use as a medicament.
45. The vector of any one of items 38 to 40, the protein of item 41, or the composition of item 42 or 43, wherein said vector, said protein or said composition is for use in treating cancer.
46. The nucleic acid for use of any one of items 1 and 3 to 36, or the method of any one of items 2 to 36, the vector for use of item 45, the protein for use of item 45, or the composition for use of item 45, wherein said treatment of cancer comprises administration of deoxythymidine.
47. The nucleic acid for use of any one of items 1, 3 to 36 and 46, or the method of any one of items 2 to 36 and 46, the vector for use of item 45 or 46, the protein for use of item 45 or 46, or the composition for use of item 45 or 46, wherein said treatment of cancer comprises administration of additional chemotherapeutic agents, surgery and/or radiotherapy.
48. The nucleic acid for use of item 47, or the method of item 47, the vector for use of item 47, the protein for use of item 47, or the composition for use of item 47, wherein said chemotherapeutic agents are Cytarabin (araC) and/or 5-Fluoruracil (5-FU).
49. The nucleic acid for use of any one of items 1, 3 to 36, 46, 47 and 48, or the method of any one of items 2 to 36, 46, 47 and 48, the vector for use of any one of items 45 to 48, the protein for use of any one of items 45 to 48, or the composition for use of any one of items 45 to 48, wherein said cancer is a solid cancer.
50. The nucleic acid for use of any one of items 1 and 3 to 36, 46, 47 and 48, or the method of any one of items 2 to 36, 46, 47 and 48, the vector for use of any one of items 45 to 48, the protein for use of any one of items 45 to 48, or the composition for use of any one of items 45 to 48, wherein said cancer is selected from the group consisting of cervical cancer, prostate carcinoma, ductal mammary carcinoma, melanoma, colon cancer, lung cancer, liver cancer, glioblastoma, and nerve cell cancer.

While the use of mutant thymidine kinase in gene therapy is a preferred aspect (and hence the use of a nucleic acid e.g. for use in treating cancer, wherein said nucleic acid comprises a nucleotide sequence, wherein said nucleotide sequence encodes a mutant thymidine kinase), the use of (a) corresponding mutant thymidine kinase protein(s) (i.e. proteins encoded by the herein provided nucleic acids) in therapy, e.g. for use in treating cancer, is also contemplated herein.

Accordingly, the present invention relates, inter alia, to the following aspects:
1. A mutant thymidine kinase for use in treating cancer.
2. A method of treating cancer, comprising administering a mutant thymidine kinase to a subject.
3. The mutant thymidine kinase for use of item 1, or the method of item 2, wherein said mutant thymidine kinase is mutant human thymidine kinase.
4. The mutant thymidine kinase for use of item 3, or the method of item 3, wherein said mutant human thymidine kinase is mutant human thymidine kinase 1.
5. The mutant thymidine kinase for use of any one of items 1, 3 and 4, or the method of any one of items 2 to 4, wherein the activity of said mutant thymidine kinase is increased compared to the activity of wildtype thymidine kinase.
6. The mutant thymidine kinase for use of item 5, or the method of item 5, wherein said activity is specific activity.
7. The mutant thymidine kinase for use of item 5 or 6, or the method of item 5 or 6, wherein said activity of said mutant thymidine kinase is at least 9-fold increased compared to the activity of wildtype thymidine kinase.
8. The mutant thymidine kinase for use of any one of items 1 and 3 to 7, or the method of any one of items 2 to 7, wherein said mutant thymidine kinase comprises one or more amino acid substitutions at positions corresponding to positions 70 to 100 of wild-type thymidine kinase.
9. The mutant thymidine kinase for use of item 8, or the method of item 8, wherein said mutant thymidine kinase comprises one or more amino acid substitutions at positions corresponding to positions 71 to 95 of wild-type thymidine kinase.
10. The mutant thymidine kinase for use of item 9, or the method of item 9, wherein said mutant thymidine kinase comprises one or more amino acid substitutions at positions corresponding to one or more positions 73, 75, 83, 84, 90 and 95 of wild-type thymidine kinase.
11. The mutant thymidine kinase for use of item 10, or the method of item 10, wherein said mutant thymidine kinase comprises one or two amino acid substitutions at positions corresponding to positions 84 and/or 90 of wild-type thymidine kinase.
12. The mutant thymidine kinase for use of any one of items 1 and 3 to 11, or the method of any one of items 2 to 11, wherein said mutant thymidine kinase comprises one or more aliphatic, nonpolar, and neutral amino acid, one or more acid/amide, acidic polar and negatively charged amino acid and/or one or more acid/amide, polar and neutral amino acid at one or more positions corresponding to positions 70 to 100 of wild-type thymidine kinase.
13. The mutant thymidine kinase for use of any one of items 1 and 3 to 12, or the method of anyone of items 2 to 12, wherein said mutant thymidine kinase comprises one or more aliphatic, nonpolar, and neutral amino acid at one or more positions corresponding to positions 73, 75, 83, 90, and 95 of wild-type thymidine kinase.
14. The mutant thymidine kinase for use of any one of items 1 and 3 to 12, or the method of any one of items 2 to 12, wherein said mutant thymidine kinase comprises one or more aliphatic, nonpolar, and neutral amino acid at one or more positions corresponding to positions 73, 83, 90, and 95 of wild-type thymidine kinase.
15. The mutant thymidine kinase for use of any one of items 1 and 3 to 12, or the method of any one of items 2 to 12, wherein said mutant thymidine kinase comprises one or more aliphatic, nonpolar, and neutral amino acid at one or more positions corresponding to positions 75 of wild-type thymidine kinase.
16. The mutant thymidine kinase for use of any one of items 1 and 3 to 12, or the method of any one of items 2 to 12, wherein said mutant thymidine kinase comprises one or more aliphatic, nonpolar, and neutral amino acid at one or more positions corresponding to position 90 of wild-type thymidine kinase.
17. The mutant thymidine kinase for use of any one of items 1 and 3 to 12, or the method of any one of items 2 to 12, wherein said mutant thymidine kinase comprises one or more acid/amide, acidic polar and negatively charged amino acid at one or more positions corresponding to positions 75, 84 and 90 of wild-type thymidine kinase.
18. The mutant thymidine kinase for use of any one of items 1 and 3 to 12, or the method of any one of items 2 to 12, wherein said mutant thymidine kinase comprises one or two acid/amide, acidic polar and negatively charged amino acid at one or two positions corresponding to positions 84 and 90 of wild-type thymidine kinase.
19. The mutant thymidine kinase for use of any one of items 1 and 3 to 12, or the method of any one of items 2 to 12, wherein said mutant thymidine kinase comprises a acid/amide, polar and neutral amino acid at a position corresponding to position 75 of wild-type thymidine kinase.
20. The mutant thymidine kinase for use of any one of items 1 and 3 to 12, or the method of any one of items 2 to 12,
   a) wherein said mutant thymidine kinase comprises an aliphatic, nonpolar, neutral amino acid at a position corresponding to position 73 of wild-type thymidine kinase;
   b) wherein said mutant thymidine kinase comprises an aliphatic, nonpolar, neutral amino acid or an acid/amide, polar, neutral amino acid or an acid/amide, acidic polar, negatively charged amino acid at a position corresponding to position 75 of wild-type thymidine kinase;
   c) wherein said mutant thymidine kinase comprises an aliphatic, nonpolar, neutral amino acid at a position corresponding to position 83 of wild-type thymidine kinase;
   d) wherein said mutant thymidine kinase comprises an acid/amide, acidic polar, negatively charged amino acid at a position corresponding to position 84 of wild-type thymidine kinase;
   e) wherein said mutant thymidine kinase comprises an aliphatic, nonpolar, neutral amino acid or an acid/amide, acidic polar, negatively charged amino acid at a position corresponding to position 90 of wild-type thymidine kinase; and/or f) wherein said mutant thymidine kinase comprises an aliphatic, nonpolar, neutral amino acid at a position corresponding to position 95 of wild-type thymidine kinase.
21. The mutant thymidine kinase for use of any one of items 1 and 3 to 20, or the method of any one of items 2 to 20, wherein said mutant thymidine kinase comprises
   a) aliphatic, nonpolar, and neutral amino acids at positions corresponding to positions 73, 83, 90 and 95 of wild-type thymidine kinase and an acid/amide, acidic polar and negatively charged amino acid at a position corresponding to position 75 of wild-type thymidine kinase;
   b) aliphatic, nonpolar, and neutral amino acids at positions corresponding to positions 73, 83, and 90 of wild-type thymidine kinase and an acid/amide, acidic polar and negatively charged amino acid at a position corresponding to position 75 of wild-type thymidine kinase;
   c) an acid/amide, acidic polar and negatively charged amino acid at a position corresponding to position 84 of wild-type thymidine kinase and an aliphatic, nonpolar, and neutral amino acid at a position corresponding to position 90 of wild-type thymidine kinase;
   d) aliphatic, nonpolar, and neutral amino acids at positions corresponding to positions 73, 90 and 95 of wild-type thymidine kinase and an acid/amide, acidic polar and negatively charged amino acid at a position corresponding to position 75 of wild-type thymidine kinase;
   e) aliphatic, nonpolar, and neutral amino acids at positions corresponding to positions 73 and 90 of wild-type thymidine kinase and an acid/amide, acidic polar and negatively charged amino acid at a position corresponding to position 75 of wild-type thymidine kinase;
   f) an aliphatic, nonpolar, and neutral amino acid at a position corresponding to position 90 of wild-type thymidine kinase;
   g) acid/amide, acidic polar and negatively charged amino acids at positions corresponding to positions 75 and 90 of wild-type thymidine kinase;
   h) an acid/amide, acidic polar and negatively charged amino acid at a position corresponding to position 84 of wild-type thymidine kinase and an aliphatic, nonpolar, and neutral amino acid at position corresponding to position 90 of wild-type thymidine kinase;
   i) aliphatic, nonpolar, and neutral amino acids at positions corresponding to positions 75 and 90 of wild-type thymidine kinase and an acid/amide, acidic polar and negatively charged amino acid at a position corresponding to position 84 of wild-type thymidine kinase;
   j) an acid/amide, acidic polar and negatively charged amino acid at a position corresponding to position 90 of wild-type thymidine kinase;
   k) an aliphatic, nonpolar, and neutral amino acid at a position corresponding to position 90 of wild-type thymidine kinase; or
   l) an acid/amide, polar and neutral amino acid at a position corresponding to position 75 of wild-type thymidine kinase and an acid/amide, acidic polar and negatively charged amino acid at a position corresponding to position 84 of wild-type thymidine kinase.
22. The mutant thymidine kinase for use of any one of items 1 and 3 to 20, or the method of any one of items 2 to 20, wherein said mutant thymidine kinase comprises
   a) an aliphatic, nonpolar, and neutral amino acid at a position corresponding to position 90 of wild-type thymidine kinase; or
   b) an acid/amide, acidic polar and negatively charged amino acid at a position corresponding to position 84 of wild-type thymidine kinase and an aliphatic, nonpolar, and neutral amino acid at a position corresponding to position 90 of wild-type thymidine kinase.
23. The mutant thymidine kinase for use of any one of items 12 to 16 and 20 to 22, or the method of any one of items 12 to 16 and 20 to 22, wherein said aliphatic, nonpolar, and neutral amino acid is one or more of alanine, glycine, valine, isoleucine, leucine and methionine.
24. The mutant thymidine kinase for use of any one of items 12 to 14, 20a), 20c), 20e) and 20f), or the method of any one of items 12 to 14, 20a), 20c), 20e) and 20f), wherein said aliphatic, nonpolar, and neutral amino acid is alanine.
25. The mutant thymidine kinase for use of any one of items 12, 13, 15 and 20b), or the method of any one of items 12, 13, 15 and 20b), wherein said aliphatic, nonpolar, and neutral amino acid is glycine.
26. The mutant thymidine kinase for use of any one of items 12, 13, 16 and 20e), or the method of any one of items 12, 13, 16 and 20e), wherein said aliphatic, nonpolar, and neutral amino acid is valine.
27. The mutant thymidine kinase for use of any one of items 12, 17, 18, and 20 to 22, or the method of any one of items 12, 17, 18, and 20 to 22, wherein said acid/amide, acidic polar and negatively charged amino acid is one or more of aspartic acid and glutamic acid.
28. The mutant thymidine kinase for use of any one of items 12, 17, 20b), 20d), 20e), 21a), 21b), 21d), 21e), 21g), 21h) and 21j), or the method of any one of items 12, 17, 20b), 20d), 20e), 21a), 21b), 21d), 21e), 21g), 21h) and 21j), wherein said acid/amide, acidic polar and negatively charged amino acid is aspartic acid.
29. The mutant thymidine kinase for use of any one of items 12, 18, 20d), 20e), 21c), 21g), 21i) and 21l), or the method of any one of items 12, 18, 20d), 20e), 21c), 21g), 21i) and 21l), wherein said acid/amide, acidic polar and negatively charged amino acid is glutamic acid.
30. The mutant thymidine kinase for use of any one of items 12 and 19 to 21, or the method of any one of items 12 and 19 to 21, wherein said acid/amide, polar and neutral amino acid is one or more of glutamine, asparagine, histidine, serine, threonine, tyrosine, and cysteine.
31. The mutant thymidine kinase for use of any one of items 12, 19, 20b) and 21l), or the method of any one of items 12, 19, 20b) and 21l), wherein said acid/amide, polar and neutral amino acid is glutamine.
32. The mutant thymidine kinase for use of any one of items 1 and 3 to 31, or the method of anyone of items 2 to 31, wherein said mutant thymidine kinase comprises
   a) alanine at positions corresponding to positions 73, 83, 90 and 95 of wild-type thymidine kinase and aspartic acid at a position corresponding to position 75 of wild-type thymidine kinase;
   b) alanine at positions corresponding to positions 73, 83, and 90 of wild-type thymidine kinase and aspartic acid at a position corresponding to position 75 of wild-type thymidine kinase;
   c) glutamic acid at a position corresponding to position 84 of wild-type thymidine kinase and alanine at a position corresponding to position 90 of wild-type thymidine kinase;
   d) alanine at positions corresponding to positions 73, 90 and 95 of wild-type thymidine kinase and aspartic acid at a position corresponding to position 75 of wild-type thymidine kinase;
   e) alanine at positions corresponding to positions 73, and 90 of wild-type thymidine kinase and aspartic acid at a position corresponding to position 75 of wild-type thymidine kinase;
   f) alanine at a position corresponding to position 90 of wild-type thymidine kinase; g) aspartic acid at a position corresponding to position 75 of wild-type thymidine kinase and glutamic acid at a position corresponding to position 90 of wild-type thymidine kinase;
   h) aspartic acid at a position corresponding to position 84 of wild-type thymidine kinase and alanine at position corresponding to position 90 of wild-type thymidine kinase;
   i) glycine at a position corresponding to position 75 of wild-type thymidine kinase, glutamic acid at a position corresponding to position 84 of wild-type thymidine kinase and alanine at a position corresponding to position 90 of wild-type thymidine kinase;
   j) aspartic acid at a position corresponding to position 90 of wild-type thymidine kinase;
   k) valine at a position corresponding to position 90 of wild-type thymidine kinase; or
   l) glutamine at a position corresponding to position 75 of wild-type thymidine kinase and glutamic acid at a position corresponding to position 84 of wild-type thymidine kinase.
33. The mutant thymidine kinase for use of any one of items 1 and 3 to 32, or the method of any one of items 2 to 32, wherein said mutant thymidine kinase comprises
   a) alanine at a position corresponding to position 90 of wild-type thymidine kinase; or b) aspartic acid amino acid at a position corresponding to position 84 of wild-type thymidine kinase and an alanine at a position corresponding to position 90 of wild-type thymidine kinase.
34. The mutant thymidine kinase for use of any one of items 1 and 3 to 33, or the method of any one of items 2 to 33, wherein said wild-type thymidine kinase is a wild-type human thymidine kinase, preferably wild-type human thymidine kinase 1.
35. The mutant thymidine kinase for use of item 34, or the method of item 34, wherein said human wild-type thymidine kinase has an amino acid sequence shown in SEQ ID NO. 28.
36. The mutant thymidine kinase for use of any one of items 1 and 3 to 35, or the method of any one of items 2 to 35, wherein said mutant thymidine kinase is selected from the group consisting of:
   a) a mutant thymidine kinase comprising an amino acid sequence as depicted in any one of SEQ ID NOs: 2, 4, 6, 8, 10, 12, 14, 16, 18, 20, 22 and 24;
   b) a mutant thymidine kinase comprising an amino acid sequence encoded by a nucleic acid comprising a nucleotide sequence as depicted in SEQ ID NO: 1, 3, 5, 7, 9, 11, 13, 15, 17, 19, 21 and 23;
   (c) a mutant thymidine kinase comprising an amino acid sequence encoded by a nucleic acid hybridizing under stringent conditions to the complementary strand of nucleic acids as defined in (b);
   (d) a mutant thymidine kinase having at least 70% identity to the mutant thymidine kinase of any one of (a) to (c); and
   (e) a mutant thymidine kinase comprising an amino acid sequence encoded by a nucleic acid being degenerate as a result of the genetic code to the nucleotide sequence of a nucleic acid as defined in (b) or (c).
37. The mutant thymidine kinase for use of any one of items 1 and 3 to 35, or the method of any one of items 2 to 35, wherein said mutant thymidine kinase is selected from the group consisting of:
   a) a mutant thymidine kinase comprising an amino acid sequence as depicted in any one of SEQ ID NOs: 10 and 12;
   b) a mutant thymidine kinase comprising an amino acid sequence encoded by a nucleic acid comprising a nucleotide sequence as depicted in SEQ ID NO: 9 and 11;
   c) a mutant thymidine kinase comprising an amino acid sequence encoded by a nucleic acid hybridizing under stringent conditions to the complementary strand of nucleic acids as defined in (b);
   (d) a mutant thymidine kinase having at least 70% identity to the mutant thymidine kinase of any one of (a) to (c); and
   (e) a mutant thymidine kinase comprising an amino acid sequence encoded by a nucleic acid being degenerate as a result of the genetic code to the nucleotide sequence of a nucleic acid as defined in (b) or (c).
38. The mutant thymidine kinase for use of any one of items 1 and 3 to 37, or the method of any one of items 2 to 37, wherein said mutant thymidine kinase is a mutant thymidine kinase protein.
39. The mutant thymidine kinase for use of any one of items 1 and 3 to 38, or the method of any one of items 2 to 38, wherein said treatment of cancer comprises administration of deoxythymidine.
40. The mutant thymidine kinase for use of any one of items 1, and 3 to 39, or the method of any one of items 2 to 39, wherein said treatment of cancer comprises administration of additional chemotherapeutic agents, surgery and/or radiotherapy.
41. The mutant thymidine kinase for use of item 40, or the method of item 40, wherein said chemotherapeutic agents are Cytarabin (araC) and/or 5-Fluoruracil (5-FU).
42. The mutant thymidine kinase for use of any one of items 1, and 3 to 41, or the method of any one of items 2 to 41, wherein said cancer is a solid cancer.
43. The mutant thymidine kinase for use of any one of items 1 and 3 to 41, or the method of any one of items 2 to 41, wherein said cancer is selected from the group consisting of cervical cancer, prostate carcinoma, ductal mammary carcinoma, melanoma, colon cancer, lung cancer, liver cancer, glioblastoma, and nerve cell cancer.

Herein provided are nucleic acids and proteins of (a) mutant thymidine kinase(s) that may be advantageously used in the herein disclosed cancer therapy.

Accordingly, the present invention relates, inter alia, to the following aspects:
1. A nucleic acid, wherein said nucleic acid comprises a nucleotide sequence, wherein said nucleotide sequence encodes a mutant thymidine kinase.
2. The nucleic acid of item 1, wherein said mutant thymidine kinase is mutant human thymidine kinase.
3. The nucleic acid of item 2, wherein said mutant human thymidine kinase is mutant human thymidine kinase 1.
4. The nucleic acid of any one of items 1 to 3, wherein the activity of said mutant thymidine kinase is increased compared to the activity of wildtype thymidine kinase.
5. The nucleic acid of item 4, wherein said activity is specific activity.
6. The nucleic acid of item 4 or 5, wherein said activity of said mutant thymidine kinase is at least 9-fold increased compared to the activity of wildtype thymidine kinase.
7. The nucleic acid of any one of items 1 to 6, wherein said mutant thymidine kinase comprises one or more amino acid substitutions at positions corresponding to positions 70 to 100 of wild-type thymidine kinase.
8. The nucleic acid of item 7, wherein said mutant thymidine kinase comprises one or more amino acid substitutions at positions corresponding to positions 71 to 95 of wild-type thymidine kinase.
9. The nucleic acid of item 8, wherein said mutant thymidine kinase comprises one or more amino acid substitutions at positions corresponding to one or more positions 73, 75, 83, 84, 90 and 95 of wild-type thymidine kinase.
10. The nucleic acid of item 9, wherein said mutant thymidine kinase comprises one or two amino acid substitutions at positions corresponding to positions 84 and/or 90 of wild-type thymidine kinase.
11. The nucleic acid of any one of items 1 to 10, wherein said mutant thymidine kinase comprises one or more aliphatic, nonpolar, and neutral amino acid, one or more acid/amide, acidic polar and negatively charged amino acid and/or one or more acid/amide, polar and neutral amino acid at one or more positions corresponding to positions 70 to 100 of wild-type thymidine kinase.
12. The nucleic acid of any one of items 1 to 11, wherein said mutant thymidine kinase comprises one or more aliphatic, nonpolar, and neutral amino acid at one or more positions corresponding to positions 73, 75, 83, 90, and 95 of wild-type thymidine kinase.
13. The nucleic acid of any one of items 1 to 11, wherein said mutant thymidine kinase comprises one or more aliphatic, nonpolar, and neutral amino acid at one or more positions corresponding to positions 73, 83, 90, and 95 of wild-type thymidine kinase.
14. The nucleic acid of any one of items 1 to 11, wherein said mutant thymidine kinase comprises one or more aliphatic, nonpolar, and neutral amino acid at one or more positions corresponding to positions 75 of wild-type thymidine kinase.
15. The nucleic acid of any one of items 1 to 11, wherein said mutant thymidine kinase comprises one or more aliphatic, nonpolar, and neutral amino acid at one or more positions corresponding to position 90 of wild-type thymidine kinase.
16. The nucleic acid of any one of items 1 to 11, wherein said mutant thymidine kinase comprises one or more acid/amide, acidic polar and negatively charged amino acid at one or more positions corresponding to positions 75, 84 and 90 of wild-type thymidine kinase.
17. The nucleic acid of any one of items 1 11, wherein said mutant thymidine kinase comprises one or two acid/amide, acidic polar and negatively charged amino acid at one or two positions corresponding to positions 84 and 90 of wild-type thymidine kinase.
18. The nucleic acid of any one of items 1 to 11, wherein said mutant thymidine kinase comprises a acid/amide, polar and neutral amino acid at a position corresponding to position 75 of wild-type thymidine kinase.
19. The nucleic acid of any one of items 1 to 11,
   a) wherein said mutant thymidine kinase comprises an aliphatic, nonpolar, neutral amino acid at a position corresponding to position 73 of wild-type thymidine kinase;
   b) wherein said mutant thymidine kinase comprises an aliphatic, nonpolar, neutral amino acid or an acid/amide, polar, neutral amino acid or an acid/amide, acidic polar, negatively charged amino acid at a position corresponding to position 75 of wild-type thymidine kinase;
   c) wherein said mutant thymidine kinase comprises an aliphatic, nonpolar, neutral amino acid at a position corresponding to position 83 of wild-type thymidine kinase;
   d) wherein said mutant thymidine kinase comprises an acid/amide, acidic polar, negatively charged amino acid at a position corresponding to position 84 of wild-type thymidine kinase;
   e) wherein said mutant thymidine kinase comprises an aliphatic, nonpolar, neutral amino acid or an acid/amide, acidic polar, negatively charged amino acid at a position corresponding to position 90 of wild-type thymidine kinase; and/or f) wherein said mutant thymidine kinase comprises an aliphatic, nonpolar, neutral amino acid at a position corresponding to position 95 of wild-type thymidine kinase.
20. The nucleic acid of any one of items 1 to 19, wherein said mutant thymidine kinase comprises
   a) aliphatic, nonpolar, and neutral amino acids at positions corresponding to positions 73, 83, 90 and 95 of wild-type thymidine kinase and an acid/amide, acidic polar and negatively charged amino acid at a position corresponding to position 75 of wild-type thymidine kinase;
   b) aliphatic, nonpolar, and neutral amino acids at positions corresponding to positions 73, 83, and 90 of wild-type thymidine kinase and an acid/amide, acidic polar and negatively charged amino acid at a position corresponding to position 75 of wild-type thymidine kinase;
   c) an acid/amide, acidic polar and negatively charged amino acid at a position corresponding to position 84 of wild-type thymidine kinase and an aliphatic, nonpolar, and neutral amino acid at a position corresponding to position 90 of wild-type thymidine kinase;
   d) aliphatic, nonpolar, and neutral amino acids at positions corresponding to positions 73, 90 and 95 of wild-type thymidine kinase and an acid/amide, acidic polar and negatively charged amino acid at a position corresponding to position 75 of wild-type thymidine kinase;
   e) aliphatic, nonpolar, and neutral amino acids at positions corresponding to positions 73 and 90 of wild-type thymidine kinase and an acid/amide, acidic polar and negatively charged amino acid at a position corresponding to position 75 of wild-type thymidine kinase;
   f) an aliphatic, nonpolar, and neutral amino acid at a position corresponding to position 90 of wild-type thymidine kinase;
   g) acid/amide, acidic polar and negatively charged amino acids at positions corresponding to positions 75 and 90 of wild-type thymidine kinase;
   h) an acid/amide, acidic polar and negatively charged amino acid at a position corresponding to position 84 of wild-type thymidine kinase and an aliphatic, nonpolar, and neutral amino acid at position corresponding to position 90 of wild-type thymidine kinase;
   i) aliphatic, nonpolar, and neutral amino acids at positions corresponding to positions 75 and 90 of wild-type thymidine kinase and an acid/amide, acidic polar and negatively charged amino acid at a position corresponding to position 84 of wild-type thymidine kinase;
   j) an acid/amide, acidic polar and negatively charged amino acid at a position corresponding to position 90 of wild-type thymidine kinase;
   k) an aliphatic, nonpolar, and neutral amino acid at a position corresponding to position 90 of wild-type thymidine kinase; or
   l) an acid/amide, polar and neutral amino acid at a position corresponding to position 75 of wild-type thymidine kinase and an acid/amide, acidic polar and negatively charged amino acid at a position corresponding to position 84 of wild-type thymidine kinase.
21. The nucleic acid of any one of items 1 to 19, wherein said mutant thymidine kinase comprises
   a) an aliphatic, nonpolar, and neutral amino acid at a position corresponding to position 90 of wild-type thymidine kinase; or
   b) an acid/amide, acidic polar and negatively charged amino acid at a position corresponding to position 84 of wild-type thymidine kinase and an aliphatic, nonpolar, and neutral amino acid at a position corresponding to position 90 of wild-type thymidine kinase.
22. The nucleic acid of any one of items 11 to 15 and 19 to 21, wherein said aliphatic, nonpolar, and neutral amino acid is one or more of alanine, glycine, valine, isoleucine, leucine and methionine.
23. The nucleic acid of any one of items 11 to 13, 19a), 19c), 19e) and 19f), wherein said aliphatic, nonpolar, and neutral amino acid is alanine.
24. The nucleic acid of any one of items 11, 12, 14 and 19b), wherein said aliphatic, nonpolar, and neutral amino acid is glycine.
25. The nucleic acid of any one of items 11, 12, 15 and 19e), wherein said aliphatic, nonpolar, and neutral amino acid is valine.
26. The nucleic acid of any one of items 11, 16, 17, and 19 to 21, wherein said acid/amide, acidic polar and negatively charged amino acid is one or more of aspartic acid and glutamic acid.
27. The nucleic acid of any one of items 11, 16, 19b), 19d), 19e), 20a), 20b), 20d), 20e), 20g), 20h) and 20j), wherein said acid/amide, acidic polar and negatively charged amino acid is aspartic acid.
28. The nucleic acid of any one of items 11, 17, 19d), 19e), 20c), 20g), 20i) and 20l), wherein said acid/amide, acidic polar and negatively charged amino acid is glutamic acid.
29. The nucleic acid of any one of items 11 and 18 to 20, wherein said acid/amide, polar and neutral amino acid is one or more of glutamine, asparagine, histidine, serine, threonine, tyrosine, and cysteine.
30. The nucleic acid of any one of items 11, 18, 19b) and 20l), wherein said acid/amide, polar and neutral amino acid is glutamine.
31. The nucleic acid of any one of items 1 to 30, wherein said mutant thymidine kinase comprises
   a) alanine at positions corresponding to positions 73, 83, 90 and 95 of wild-type thymidine kinase and aspartic acid at a position corresponding to position 75 of wild-type thymidine kinase;
   b) alanine at positions corresponding to positions 73, 83, and 90 of wild-type thymidine kinase and aspartic acid at a position corresponding to position 75 of wild-type thymidine kinase;
   c) glutamic acid at a position corresponding to position 84 of wild-type thymidine kinase and alanine at a position corresponding to position 90 of wild-type thymidine kinase;
   d) alanine at positions corresponding to positions 73, 90 and 95 of wild-type thymidine kinase and aspartic acid at a position corresponding to position 75 of wild-type thymidine kinase;
   e) alanine at positions corresponding to positions 73, and 90 of wild-type thymidine kinase and aspartic acid at a position corresponding to position 75 of wild-type thymidine kinase;
   f) alanine at a position corresponding to position 90 of wild-type thymidine kinase; g) aspartic acid at a position corresponding to position 75 of wild-type thymidine kinase and glutamic acid at a position corresponding to position 90 of wild-type thymidine kinase;
   h) aspartic acid at a position corresponding to position 84 of wild-type thymidine kinase and alanine at position corresponding to position 90 of wild-type thymidine kinase;
   i) glycine at a position corresponding to position 75 of wild-type thymidine kinase, glutamic acid at a position corresponding to position 84 of wild-type thymidine kinase and alanine at a position corresponding to position 90 of wild-type thymidine kinase;
   j) aspartic acid at a position corresponding to position 90 of wild-type thymidine kinase;
   k) valine at a position corresponding to position 90 of wild-type thymidine kinase; or
   l) glutamine at a position corresponding to position 75 of wild-type thymidine kinase and glutamic acid at a position corresponding to position 84 of wild-type thymidine kinase.
32. The nucleic acid of any one of items 1 to 31, wherein said mutant thymidine kinase comprises
   a) alanine at a position corresponding to position 90 of wild-type thymidine kinase; or
   b) aspartic acid amino acid at a position corresponding to position 84 of wild-type thymidine kinase and an alanine at a position corresponding to position 90 of wild-type thymidine kinase.
33. The nucleic acid of any one of items 1 to 32, wherein said wild-type thymidine kinase is a wild-type human thymidine kinase, preferably wild-type human thymidine kinase 1.
34. The nucleic acid of item 33, wherein said human wild-type thymidine kinase has an amino acid sequence shown in SEQ ID NO. 28.
35. The nucleic acid of any one of items 1 to 34, wherein said nucleic acid is selected from the group consisting of:
   a) a nucleic acid comprising a nucleotide sequence, wherein said nucleotide sequence encodes a mutant thymidine kinase, wherein said mutant thymidine kinase comprises an amino acid sequence as depicted in any one of SEQ ID NOs: 2, 4, 6, 8, 10, 12, 14, 16, 18, 20, 22 and 24;
   b) a nucleic acid comprising a nucleotide sequence, wherein said nucleotide sequence encodes a mutant thymidine kinase, and wherein said nucleotide sequence is depicted in SEQ ID NO: 1, 3, 5, 7, 9, 11, 13, 15, 17, 19, 21 and 23;
   c) a nucleic acid hybridizing under stringent conditions to the complementary strand of the nucleic acid as defined in (a) or (b) and wherein said nucleic acid comprises a nucleotide sequence wherein said nucleotide sequence encodes a mutant thymidine kinase;
   d) a nucleic acid comprising a nucleotide sequence with at least 70% identity to the nucleotide sequence of the nucleic acids of any one of (a) to (c) and wherein said nucleotide sequence encodes a mutant thymidine kinase; and
   e) a nucleic acid comprising a nucleotide sequence which is degenerate as a result of the genetic code to the nucleotide sequence of a nucleic acid of any one of (a) to (d) and wherein said nucleotide sequence encodes a mutant thymidine kinase.
36. The nucleic acid of any one of items 1 to 34, wherein said nucleic acid is selected from the group consisting of:
   a) a nucleic acid comprising a nucleotide sequence, wherein said nucleotide sequence encodes a mutant thymidine kinase, wherein said mutant thymidine kinase comprises an amino acid sequence as depicted in any one of SEQ ID NOs: 10 and 12;
   b) a nucleic acid comprising a nucleotide sequence, wherein said nucleotide sequence encodes a mutant thymidine kinase, and wherein nucleotide sequence is depicted in SEQ ID NO: 9 and 11;
   c) a nucleic acid hybridizing under stringent conditions to the complementary strand of the nucleic acid as defined in (a) or (b) and wherein said nucleic acid comprises a nucleotide sequence wherein said nucleotide sequence encodes a mutant thymidine kinase;
   d) a nucleic acid comprising a nucleotide sequence with at least 70% identity to the nucleotide sequence of the nucleic acids of any one of (a) to (c) and wherein said nucleotide sequence encodes a mutant thymidine kinase; and
   e) a nucleic acid comprising a nucleotide sequence which is degenerate as a result of the genetic code to the nucleotide sequence of a nucleic acid of any one of (a) to (d) and wherein said nucleotide sequence encodes a mutant thymidine kinase.
37. Vector comprising the nucleic acid of any one of items 1 to 36.
38. The vector of item 37, wherein said vector is a gene therapy vector.
39. The vector of item 37 or 38, wherein said vector is an AAV vector, adenovirus vector, or a lentivirus vector.
40. Composition comprising the nucleic acid of any one of items 1 to 36, or comprising a vector of any one of items 37 to 39.
41. The composition of item 40, wherein said composition is a pharmaceutical composition.
42. The nucleic acid of any one of items 1 to 36, the vector of any one of items 37 to 39, or the composition of item 40 or 41, wherein said nucleic acid, said vector, or said composition is for use as a medicament.
43. The vector of any one of items 37 to 39, or the composition of item 40 or 41, wherein said vector, or said composition is for use in treating cancer.
44. The vector for use of item 43, or the composition for use of item 43, wherein said treatment of cancer comprises administration of deoxythymidine.
45. The vector for use of item 43 or 44, or the composition for use of item 43 or 44, wherein said treatment of cancer comprises administration of additional chemotherapeutic agents, surgery and/or radiotherapy.
46. The vector for use of item 45, or the composition for use of item 45, wherein said chemotherapeutic agents are Cytarabin (araC) and/or 5-Fluoruracil (5-FU).
47. The vector for use of any one of items 43 to 46, or the composition for use of any one of items 43 to 46, wherein said cancer is a solid cancer.
48. The vector for use of any one of items 43 to 46, or the composition for use of any one of items 43 to 46, wherein said cancer is selected from the group consisting of cervical cancer, prostate carcinoma, ductal mammary carcinoma, melanoma, colon cancer, lung cancer, liver cancer, glioblastoma, and nerve cell cancer.

In a further aspect, the present invention provides mutant thymidine kinase proteins.
1. A mutant thymidine kinase.
2. The mutant thymidine kinase of item 1, wherein said mutant thymidine kinase is mutant human thymidine kinase.
3. The mutant thymidine kinase item 2, wherein said mutant human thymidine kinase is mutant human thymidine kinase 1.
4. The mutant thymidine kinase of any one of items 1 to 3, wherein the activity of said mutant thymidine kinase is increased compared to the activity of wildtype thymidine kinase.
5. The mutant thymidine kinase of item 4, wherein said activity is specific activity.
6. The mutant thymidine kinase 4 or 5, wherein said activity of said mutant thymidine kinase is at least 9-fold increased compared to the activity of wildtype thymidine kinase.
7. The mutant thymidine kinase of any one of items 1 to 6, wherein said mutant thymidine kinase comprises one or more amino acid substitutions at positions corresponding to positions 70 to 100 of wild-type thymidine kinase.
8. The mutant thymidine kinase of item 7, wherein said mutant thymidine kinase comprises one or more amino acid substitutions at positions corresponding to positions 71 to 95 of wild-type thymidine kinase.
9. The mutant thymidine kinase of item 8, wherein said mutant thymidine kinase comprises one or more amino acid substitutions at positions corresponding to one or more positions 73, 75, 83, 84, 90 and 95 of wild-type thymidine kinase.
10. The mutant thymidine kinase of item 9, wherein said mutant thymidine kinase comprises one or two amino acid substitutions at positions corresponding to positions 84 and/or 90 of wild-type thymidine kinase.
11. The mutant thymidine kinase of any one of items 1 to 10, wherein said mutant thymidine kinase comprises one or more aliphatic, nonpolar, and neutral amino acid, one or more acid/amide, acidic polar and negatively charged amino acid and/or one or more acid/amide, polar and neutral amino acid at one or more positions corresponding to positions 70 to 100 of wild-type thymidine kinase.
12. The mutant thymidine kinase of any one of items 1 to 11, wherein said mutant thymidine kinase comprises one or more aliphatic, nonpolar, and neutral amino acid at one or more positions corresponding to positions 73, 75, 83, 90, and 95 of wild-type thymidine kinase.
13. The mutant thymidine kinase of any one of items 1 to 11, wherein said mutant thymidine kinase comprises one or more aliphatic, nonpolar, and neutral amino acid at one or more positions corresponding to positions 73, 83, 90, and 95 of wild-type thymidine kinase.
14. The mutant thymidine kinase of any one of items 1 to 11, wherein said mutant thymidine kinase comprises one or more aliphatic, nonpolar, and neutral amino acid at one or more positions corresponding to positions 75 of wild-type thymidine kinase.
15. The mutant thymidine kinase of any one of items 1 to 11, wherein said mutant thymidine kinase comprises one or more aliphatic, nonpolar, and neutral amino acid at one or more positions corresponding to position 90 of wild-type thymidine kinase.
16. The mutant thymidine kinase of any one of items 1 to 11, wherein said mutant thymidine kinase comprises one or more acid/amide, acidic polar and negatively charged amino acid at one or more positions corresponding to positions 75, 84 and 90 of wild-type thymidine kinase.
17. The mutant thymidine kinase of any one of items 1 to 11, wherein said mutant thymidine kinase comprises one or two acid/amide, acidic polar and negatively charged amino acid at one or two positions corresponding to positions 84 and 90 of wild-type thymidine kinase.
18. The mutant thymidine kinase of any one of items 1 to 11, wherein said mutant thymidine kinase comprises a acid/amide, polar and neutral amino acid at a position corresponding to position 75 of wild-type thymidine kinase.
19. The mutant thymidine kinase of any one of items 1 to 11,
   a) wherein said mutant thymidine kinase comprises an aliphatic, nonpolar, neutral amino acid at a position corresponding to position 73 of wild-type thymidine kinase;
   b) wherein said mutant thymidine kinase comprises an aliphatic, nonpolar, neutral amino acid or an acid/amide, polar, neutral amino acid or an acid/amide, acidic polar, negatively charged amino acid at a position corresponding to position 75 of wild-type thymidine kinase;
   c) wherein said mutant thymidine kinase comprises an aliphatic, nonpolar, neutral amino acid at a position corresponding to position 83 of wild-type thymidine kinase;
   d) wherein said mutant thymidine kinase comprises an acid/amide, acidic polar, negatively charged amino acid at a position corresponding to position 84 of wild-type thymidine kinase;
   e) wherein said mutant thymidine kinase comprises an aliphatic, nonpolar, neutral amino acid or an acid/amide, acidic polar, negatively charged amino acid at a position corresponding to position 90 of wild-type thymidine kinase; and/or
   f) wherein said mutant thymidine kinase comprises an aliphatic, nonpolar, neutral amino acid at a position corresponding to position 95 of wild-type thymidine kinase.
20. The mutant thymidine kinase of any one of items 1 to 19, wherein said mutant thymidine kinase comprises
   a) aliphatic, nonpolar, and neutral amino acids at positions corresponding to positions 73, 83, 90 and 95 of wild-type thymidine kinase and an acid/amide, acidic polar and negatively charged amino acid at a position corresponding to position 75 of wild-type thymidine kinase;
   b) aliphatic, nonpolar, and neutral amino acids at positions corresponding to positions 73, 83, and 90 of wild-type thymidine kinase and an acid/amide, acidic polar and negatively charged amino acid at a position corresponding to position 75 of wild-type thymidine kinase;
   c) an acid/amide, acidic polar and negatively charged amino acid at a position corresponding to position 84 of wild-type thymidine kinase and an aliphatic, nonpolar, and neutral amino acid at a position corresponding to position 90 of wild-type thymidine kinase;
   d) aliphatic, nonpolar, and neutral amino acids at positions corresponding to positions 73, 90 and 95 of wild-type thymidine kinase and an acid/amide, acidic polar and negatively charged amino acid at a position corresponding to position 75 of wild-type thymidine kinase;
   e) aliphatic, nonpolar, and neutral amino acids at positions corresponding to positions 73 and 90 of wild-type thymidine kinase and an acid/amide, acidic polar and negatively charged amino acid at a position corresponding to position 75 of wild-type thymidine kinase;
   f) an aliphatic, nonpolar, and neutral amino acid at a position corresponding to position 90 of wild-type thymidine kinase;
   g) acid/amide, acidic polar and negatively charged amino acids at positions corresponding to positions 75 and 90 of wild-type thymidine kinase;
   h) an acid/amide, acidic polar and negatively charged amino acid at a position corresponding to position 84 of wild-type thymidine kinase and an aliphatic, nonpolar, and neutral amino acid at position corresponding to position 90 of wild-type thymidine kinase;
   i) aliphatic, nonpolar, and neutral amino acids at positions corresponding to positions 75 and 90 of wild-type thymidine kinase and an acid/amide, acidic polar and negatively charged amino acid at a position corresponding to position 84 of wild-type thymidine kinase;
   j) an acid/amide, acidic polar and negatively charged amino acid at a position corresponding to position 90 of wild-type thymidine kinase;
   k) an aliphatic, nonpolar, and neutral amino acid at a position corresponding to position 90 of wild-type thymidine kinase; or
   l) an acid/amide, polar and neutral amino acid at a position corresponding to position 75 of wild-type thymidine kinase and an acid/amide, acidic polar and negatively charged amino acid at a position corresponding to position 84 of wild-type thymidine kinase.
21. The mutant thymidine kinase of any one of items 1 to 19, wherein said mutant thymidine kinase comprises
   a) an aliphatic, nonpolar, and neutral amino acid at a position corresponding to position 90 of wild-type thymidine kinase; or
   b) an acid/amide, acidic polar and negatively charged amino acid at a position corresponding to position 84 of wild-type thymidine kinase and an aliphatic, nonpolar, and neutral amino acid at a position corresponding to position 90 of wild-type thymidine kinase.
22. The mutant thymidine kinase of any one of items 11 to 15 and 19 to 21, wherein said aliphatic, nonpolar, and neutral amino acid is one or more of alanine, glycine, valine, isoleucine, leucine and methionine.
23. The mutant thymidine kinase of any one of items 11 to 13, 19a), 19c), 19e) and 19f), wherein said aliphatic, nonpolar, and neutral amino acid is alanine.
24. The mutant thymidine kinase of any one of items 11, 12, 14 and 19b), wherein said aliphatic, nonpolar, and neutral amino acid is glycine.
25. The mutant thymidine kinase of any one of items 11, 12, 15 and 19e), wherein said aliphatic, nonpolar, and neutral amino acid is valine.
26. The mutant thymidine kinase of any one of items 11, 16, 17, and 19 to 21, wherein said acid/amide, acidic polar and negatively charged amino acid is one or more of aspartic acid and glutamic acid.
27. The mutant thymidine kinase of any one of items 11, 16, 19b), 19d), 19e), 20a), 20b), 20d), 20e), 20g), 20h) and 20j), wherein said acid/amide, acidic polar and negatively charged amino acid is aspartic acid.
28. The mutant thymidine kinase of any one of items 11, 17, 19d), 19e), 20c), 20g), 20i) and 20l), wherein said acid/amide, acidic polar and negatively charged amino acid is glutamic acid.
29. The mutant thymidine kinase of any one of items 11 and 18 to 20, wherein said acid/amide, polar and neutral amino acid is one or more of glutamine, asparagine, histidine, serine, threonine, tyrosine, and cysteine.
30. The mutant thymidine kinase of any one of items 11, 18, 19b) and 20l), wherein said acid/amide, polar and neutral amino acid is glutamine.
31. The mutant thymidine kinase of any one of items 1 to 30, wherein said mutant thymidine kinase comprises
   a) alanine at positions corresponding to positions 73, 83, 90 and 95 of wild-type thymidine kinase and aspartic acid at a position corresponding to position 75 of wild-type thymidine kinase;
   b) alanine at positions corresponding to positions 73, 83, and 90 of wild-type thymidine kinase and aspartic acid at a position corresponding to position 75 of wild-type thymidine kinase;
   c) glutamic acid at a position corresponding to position 84 of wild-type thymidine kinase and alanine at a position corresponding to position 90 of wild-type thymidine kinase;
   d) alanine at positions corresponding to positions 73, 90 and 95 of wild-type thymidine kinase and aspartic acid at a position corresponding to position 75 of wild-type thymidine kinase;
   e) alanine at positions corresponding to positions 73, and 90 of wild-type thymidine kinase and aspartic acid at a position corresponding to position 75 of wild-type thymidine kinase;
   f) alanine at a position corresponding to position 90 of wild-type thymidine kinase;
   g) aspartic acid at a position corresponding to position 75 of wild-type thymidine kinase and glutamic acid at a position corresponding to position 90 of wild-type thymidine kinase;
   h) aspartic acid at a position corresponding to position 84 of wild-type thymidine kinase and alanine at position corresponding to position 90 of wild-type thymidine kinase;
   i) glycine at a position corresponding to position 75 of wild-type thymidine kinase, glutamic acid at a position corresponding to position 84 of wild-type thymidine kinase and alanine at a position corresponding to position 90 of wild-type thymidine kinase;
   j) aspartic acid at a position corresponding to position 90 of wild-type thymidine kinase;
   k) valine at a position corresponding to position 90 of wild-type thymidine kinase; or
   l) glutamine at a position corresponding to position 75 of wild-type thymidine kinase and glutamic acid at a position corresponding to position 84 of wild-type thymidine kinase.
32. The mutant thymidine kinase of any one of items 1 to 31, wherein said mutant thymidine kinase comprises
   a) alanine at a position corresponding to position 90 of wild-type thymidine kinase; or
   b) aspartic acid amino acid at a position corresponding to position 84 of wild-type thymidine kinase and an alanine at a position corresponding to position 90 of wild-type thymidine kinase.
33. The mutant thymidine kinase of any one of items 1 to 32, wherein said wild-type thymidine kinase is a wild-type human thymidine kinase, preferably wild-type human thymidine kinase 1.
34. The mutant thymidine kinase of item 33, wherein said human wild-type thymidine kinase has an amino acid sequence shown in SEQ ID NO. 28.
35. The mutant thymidine kinase of any one of items 1 to 34, wherein said mutant thymidine kinase is selected from the group consisting of:
   a) a mutant thymidine kinase comprising an amino acid sequence as depicted in any one of SEQ ID NOs: 2, 4, 6, 8, 10, 12, 14, 16, 18, 20, 22 and 24;
   b) a mutant thymidine kinase comprising an amino acid sequence encoded by a nucleic acid comprising a nucleotide sequence as depicted in SEQ ID NO: 1, 3, 5, 7, 9, 11, 13, 15, 17, 19, 21 and 23;
   (c) a mutant thymidine kinase comprising an amino acid sequence encoded by a nucleic acid hybridizing under stringent conditions to the complementary strand of nucleic acids as defined in (b);
   (d) a mutant thymidine kinase having at least 70% identity to the mutant thymidine kinase of any one of (a) to (c); and
   (e) a mutant thymidine kinase comprising an amino acid sequence encoded by a nucleic acid being degenerate as a result of the genetic code to the nucleotide sequence of a nucleic acid as defined in (b) or (c).
36. The mutant thymidine kinase of any one of items 1 to 34, wherein said mutant thymidine kinase is selected from the group consisting of:
   a) a mutant thymidine kinase comprising an amino acid sequence as depicted in any one of SEQ ID NOs: 10 and 12;
   b) a mutant thymidine kinase comprising an amino acid sequence encoded by a nucleic acid comprising a nucleotide sequence as depicted in SEQ ID NO: 9 and 11;
   c) a mutant thymidine kinase comprising an amino acid sequence encoded by a nucleic acid hybridizing under stringent conditions to the complementary strand of nucleic acids as defined in (b);
   (d) a mutant thymidine kinase having at least 70% identity to the mutant thymidine kinase of any one of (a) to (c); and
   (e) a mutant thymidine kinase comprising an amino acid sequence encoded by a nucleic acid being degenerate as a result of the genetic code to the nucleotide sequence of a nucleic acid as defined in (b) or (c).
37. The mutant thymidine kinase of any one of items 1 to 36, wherein said mutant thymidine kinase is a mutant thymidine kinase protein.
38. Composition comprising the mutant thymidine kinase of any one of items 1 to 37.
39. The composition of item 38, wherein said composition is a pharmaceutical composition.
40. The mutant thymidine kinase of any one of items 1 to 37, or the composition of item 38 or 39, wherein said mutant thymidine kinase, or said composition is for use as a medicament.
41. The mutant thymidine kinase of any one of items 1 to 37, or the composition of item 38 or 39, wherein said mutant thymidine kinase or said composition is for use in treating cancer.
42. The mutant thymidine kinase for use of item 41, or the composition for use of item 41, wherein said treatment of cancer comprises administration of deoxythymidine.
43. The mutant thymidine kinase for use of item 41 or 429, or the composition for use of item 41 or 42, wherein said treatment of cancer comprises administration of additional chemotherapeutic agents, surgery and/or radiotherapy.
44. The mutant thymidine kinase for use of item 43, or the composition for use of item 43, wherein said chemotherapeutic agents are Cytarabin (araC) and/or 5-Fluoruracil (5-FU).
45. The mutant thymidine kinase for use of any one of items 41 to 44, or the composition for use of any one of items 41 to 44, wherein said cancer is a solid cancer.
46. The mutant thymidine kinase for use of any one of items 41 to 44, or the composition for use of any one of items 41 to 44, wherein said cancer is selected from the group consisting of cervical cancer, prostate carcinoma, ductal mammary carcinoma, melanoma, colon cancer, lung cancer, liver cancer, glioblastoma, and nerve cell cancer.

As indicated herein above, the present invention relates inter alia to a nucleic acid for use in treating cancer, wherein said nucleic acid comprises a nucleotide sequence, wherein said nucleotide sequence encodes a mutant thymidine kinase. In a related aspect, the present invention relates to the use of a nucleic acid for the preparation/manufacture of a pharmaceutical composition/medicament for the treatment of cancer, wherein said nucleic acid comprises a nucleotide sequence, wherein said nucleotide sequence encodes a mutant thymidine kinase. In a related aspect, the present invention relates to the use of a nucleic acid for the treatment of cancer wherein said nucleic acid comprises a nucleotide sequence, wherein said nucleotide sequence encodes a mutant thymidine kinase. In a related aspect, the present invention relates to a method of treating cancer, comprising administering (an effective amount of) a nucleic acid to a subject (in need of the treatment), wherein said nucleic acid comprises a nucleotide sequence, wherein said nucleotide sequence encodes a mutant thymidine kinase. The above applies mutatis mutandis to vectors, mutant thymidine kinase proteins or compositions as described herein in cancer therapy/for treating cancer.

Preferably, the mutant thymidine kinase is mutant human thymidine kinase, particularly preferably a mutant human thymidine kinase 1. In one aspect, the herein provided and to be used mutant tymidine kinase is not a mutant viral thymidine kinases, e.g. is not a mutant herpesviridae thymidine kinase. Preferably, the wild-type thymidine kinase is wild-type human thymidine kinase, particularly preferably a wild-type human thymidine kinase 1.

Wild-type thymidine kinase, e.g. human thymidine kinase like human thymidine kinase 1 are known in the art. Corresponding nucleotide sequences and amino acid sequences of wild-type thymidine kinase are, inter alia, disclosed in public databases like NCBI or EMBL, e.g. under accession numbers (Homo sapiens thymidine kinase 1 (TK1), transcript variant 1, mRNA NCBI ref. number: NM 003258) SEQ ID NO. 27 and/or 29:

(SEQ ID NO: 29)

| | |
|---|---|
| 1 | gcgcacgtcc cggattcctc ccacgagggg gcgggctgcg |
| | gccaaatctc ccgccaggtc |
| 61 | agcggccggg cgctgattgg ccccatggcg gcggggccgg |
| | ctcgtgattg gccagcacgc |
| 121 | cgtggtttaa agcggtcggc gcgggaacca ggggcttact |
| | gcgggacggc cttggagagt |
| 181 | actcgggttc gtgaacttcc cggaggcgca atgagctgca |
| | ttaacctgcc cactgtgctg |
| 241 | cctggctccc ccagcaagac ccgggggcag atccaggtga |
| | ttctcgggcc gatgttctca |
| 301 | ggaaaaagca cagagttgat gaaacgcgtc cgtcgcttcc |
| | agattgctca gtacaagtgc |
| 361 | ctggtgatca agtatgccaa agacactcgc tacaggagca |
| | gcttctgcac acatgaccgg |
| 421 | aacaccatgg aggcactgcc cgcctgcctg ctccgagacg |
| | tggcccagga ggccctgggc |
| 481 | gtggctgtca taggcatcga cgaggggcag tttttccctg |
| | acatcgtgga gttctgcgag |
| 541 | gccatggcca acgccgggaa gaccgtaatt gtggctgcac |
| | tggatgggac cttccagagg |
| 601 | aagccatttg gggccatcct gaacctggtg ccgctggccg |
| | agagcgtggt gaagctgacg |
| 661 | gcggtgtgca tggagtgctt ccggqaagcc gcctatacca |
| | agaggctcgg cacagaaaag |
| 721 | gaggtcgagg tgattggggg agcagacaag taccactccg |
| | tgtgtcggct ctgctacttc |
| 781 | aagaaggcct caggccagcc tgccgggccg gacaacaaag |
| | agaactgccc agtgccagga |
| 841 | aagccagggg aagccgtgga tgccaggaag ctctttgccc |
| | cacagcagat tctgcaatgc |
| 901 | agccatgcca actgagggac ctgcgagggc cgcccgctcc |
| | cttcctgcca ctgccgccta |
| 961 | ctggacgctg ccctgcatgc tgcccagcca ctccaggagg |
| | aagtcgggag gcgtggaggg |
| 1021 | tgaccacacc ttggccttct gggaactctc ctttgtgtgg |
| | ctgccccacc tgccgcatgc |
| 1081 | tccctcctct cctacccact ggtctgctta aagcttccct |
| | ctcagctgat gggacgatcg |
| 1141 | cccaggctgg agctggcccc gcttggtgac ctgggatctg |
| | gcacactccc tctccttggg |
| 1201 | gtgagggaca gagccccacg ctgttgacat cagcctgctt |
| | cttcccctct gcggctttca |
| 1261 | ctgctgagtt tctgttctcc ctgggaagcc tgtgccagca |
| | cctttgagcc ttggcccaca |
| 1321 | ctgaggctta ggcctctctg cctgggatgg gctcccaccc |
| | tcccctgagg atggcctgga |
| 1381 | ttcacgccct cttgtttcct tttaggctca aagcccttcc |
| | tacctctggt gatggtttcc |
| 1441 | acaggaacaa cagcatcttt caccaagatg ggtggcacca |
| | accttgctgg gacttggatc |
| 1501 | ccaggggctt atctcttcaa gtgtggaaag ggcagggtcc |
| | acgcctctgc tgtagcttat |
| 1561 | gaaattaact aattgaaaat tcactggttg gtggaaaaaa |
| | aaaaaaaaaa aaaaaa |

The amino acid sequence of an exemplary human wild-type thymidine kinase is shown in SEQ ID NO. 28 Based on thymidine kinase, cytosolic isoform 1 [Homo sapiens] NCBI ref. number: NP 003249

(SEQ ID NO: 28)

| | |
|---|---|
| 1 | mscinlptvl pgspsktrgq iqvilgpmfs gkstelmrrv |
| | rrfqiaqykc lvikyakdtr |
| 61 | ysspfcthdr ntmealpacl lrdvaqealg vavigidegq |
| | ffpdivefce amanagktvi |
| 121 | vaaldgtfqr kpfgailnlv plaesvvklt avcmecfrea |
| | aytkrlgtek eveviggadk |
| 181 | yhsvcrlcyf kkasggpagp dnkencpvpg kpgeavaark |
| | lfapqqilqc span |

The herein provided and to be used mutant thymidine kinase(s) show or have an increased activity compared to the activity of wildtype thymidine kinase. The term “activity” as used herein refers primarily to the specific enzymatic activity, defined as the activity of an enzyme per milligram of total protein (expressed in μmol min⁻¹mg⁻¹ or pmol min⁻¹mg⁻¹). In the case of the thymidine kinase this is particularly the activity/capacity to phosphorylate deoxythymidine(s) (dTh) to deoxythymidine monophosphate (dTMP). Corresponding assays are known in the art and can be used accordingly herein, see [42].

It is understood that the activity of a mutant thymidine kinase is typically compared to the activity of the corresponding wildtype thymidine kinase (i.e. the wildtype thymidine kinase from which the mutant is derived) in order to assess whether (and to which extent) the mutant has an increased activity. For example, if (a) mutation(s) has (have) been introduced into a wild-type human thymidine kinase in order to prepare a mutant human thymidine kinase in accordance with the teaching herein, the activity of the prepared mutant human thymidine kinase would typically be compared with that of the wild-type human thymidine kinase. More specifically, if (a) mutation(s) has (have) been introduced into a wild-type human thymidine kinase 1 in order to prepare a mutant human thymidine kinase 1 in accordance with the teaching herein, the activity of the prepared mutant human thymidine kinase 1 would typically be compared with that of the wild-type human thymidine kinase 1.

In a preferred aspect, the activity is the specific activity (e.g. pmol/mg·min (or pmol min⁻¹mg⁻¹) or μmol min⁻¹mg⁻¹).

As a reference point, the herein provided mutant G90A human thymidine kinase 1 may be used. As exemplified herein, said mutant has an about 9-fold specific enzyme activity compared to its wild-type counterpart, i.e. the wild-type human thymidine kinase 1 (see Example 4, Table 2) Accordingly, it is envisaged herein that the activity of the mutant thymidine kinase is at least 2-fold, 3-fold, 4-fold, 5-fold, 6-fold, 7-fold, 8-fold, preferably at least 9-fold, e.g. at least 10-fold increased compared to the activity of wildtype thymidine kinase (which would correspond to an increase in activity of at least 100%, 200%, 300%, 400%, 500%, 600%, 700%, preferably at least 800%, e.g. at least 900% of the mutant thymidine kinase compared to the activity of wildtype thymidine kinase). The specific enzyme activity is determined after a distinct point of time after the start of the reaction (e.g. determined after 5 min). The -fold increase can easily be determined by comparing the values for the mutant tymidine kinase and wild-type tymidine kinase as determined at that distinct time point.

An exemplary assay for determining the (specific) activity is described in the following.

Activity measurements of thymidine kinases can be performed using the following thymidine/deoxycytidine kinase assay:

For the determination of a specific activity a quantification of the protein solution has to be done first, e.g. according the method of Bradford. [43] The dye stock solution (Biorad) containing methanol and acetic acid is diluted 1:4 with ddH2O. An aliquot of the protein solution is then filled up to 1 ml with the diluted Bradford solution. (Usually 5 μl of protein solution are diluted in 995 ml Bradford solution. The protein concentration is calculated by multiplying the absorbance at =595 nm with 17 and dividing this by 5, resulting in the concentration in μg/μl.) Depending on the concentration of the kinases 5-20 g of the sample solution can be used.

The following components can be mixed for the enzyme assay:

10 fold mix:
0.5 M Tris-HCl, pH 7.5
0.1 M DTT
25 mM ATP
25 mM MgCl₂

Set up for the activity test: 5.0 μl 10-fold mix
2.5 μl BSA (60 mg/ml)
1.0 μl Chaps (25 mM)
1.0 μl NaF (0.15 M)
1.0 μl ³H-dTh or 3H-dCyd (500 μM, 2 Ci/mmol)
(optionally 1.0 μl 10 mM THUR, only for dCK-assay)
5.0-20 μl of the protein solution
Filled up with ddH₂O to 50 μl

The set-up is incubated at 37° C. At a distinct time point (e.g. after 5 min) 10 μl aliquots are removed from the reaction mix and pipetted on small pieces of DE81-filters. After removal of every aliquot the filter papers are washed in a big volume of ammonium formiat (5 mM for TK assay, 2 mM for other assays), transferred to water and rinsed and finally dried after shortly immersing in ethanol. Then after the dried filters are transferred into the scintillation tubes the bound radioactive nucleotides are eluted by addition of 500 μl elution buffer (0.1 M HCl, 0.2 M KCl). 2.5 ml of scintillation solution are added before measuring the radioactivity. A standard is created in order to convert the cpm-values into pmol: 5 μl from some of the reactions are removed and pipetted on DE81-filters, which are not washed, but directly transferred to the scintillation tubes, treated like described above and measured. 5 μl of the standard contained 50 pMol of the substrate or the product. From the cpm-values of these samples one got the converting factor UF (is equal to the specific activity of the used substrate): UF=cpm standard/50 pMol

It is understood that aliquots can be taken from the reaction mix at distinct time points. “Distinct time points” as used herein can mean e.g. after 5 min, 10 min, 15 min, 20 min and the like (and any integers (e.g. 1 min, 2 min, 3 min, 4 min, 5 min, 6 min, 7 min, 8 min, 9 min, 10 min, 11 min, 12 min, 13 min, 14 min, 15 min, 16 min, 17 min, 18 min, 19 min, 20 min and so forth). “After” typically means “after the start of the reaction”.

For the calculation of the whole enzymatic activity of one fraction the following formula is used:

$$U = \frac{Δ cpm . 50 μ l . Vol}{10 μ l . 20 μ l . UF}$$

10 μl are pipetted at the filter at each time point, and 20 μl of the protein solution are initially used. cpm=(cpmt2−cpm t1)/t2−t1 (cpm/min); t1=start of reaction; t2 is endpoint of reaction (e.g. at 5 min); Vol=total volume of the fraction (μl); UF is the specific activity (see above) (cpm/pMol); U is the whole activity of the enzyme per fraction (pMol/min×μl=Mol/min×ml). With the concentration determined according to Bradford above, the specific activity in pmol/mg×min can be determined.

As explained herein, a region approx. between positions 70 to 110 of wild-type thymidine kinase more specifically between positions 71, 72, 73, 74, 75 or 76 to positions 110, 109, 108, 107, 106, 105, 104, 103, 102, 101 or 100 of wild-type thymidine kinase is very sensitive for influencing the specific activity of the kinase, specifically the region corresponding to positions 70 to 100 of wild-type thymidine kinase. As shown herein a point mutation at position 90 in this region (A90G) increased the activity more than 9 fold compared to the wild type kinase. Also other mutant kinases having point mutations in this region, e.g. the double mutant V84DG90A, showed an at least 9 fold increase compared to the activity of wild type tymidine kinase. Yet, not all mutant kinases having point mutations in this region shown an increase in activity. For example, double mutant L8081F (termed herein “feeble” tymidine kinase) entailed a strong decrease of activity.

The bottom line is: an increase in activity of mutant thymidine kinases correlates with an increase in helicity in the above region of the mutant thymidine kinases (compared to wild-type thymidine kinase). Vice versa, a decrease in activity of mutant thymidine kinases correlates with a decrease in helicity in the above region of the of mutant thymidine kinases (compared to wild-type thymidine kinase). Corresponding helicity calculations can be readily performed by available tools e.g. by GOR secondary structure prediction method version IV provided by a network platform. [44] The helicity calculations according to GOR IV can be performed e.g. by Network Protein Sequence Analysis, provided from the PBIL-IBCP Institute of Biology and Protein Chemistry 7, passage of Vercors 69367 Lyon Cedex 07, FRANCE TIBS [41]; see e.g. https://npsa-prabi.ibcp.fr/cgibin/npsa_automat.pl?page=/NPSA/npsa_seccons.html

By applying these selection rules, (a) mutant thymidine kinase(s) can be screened in silico in order to determine whether the mutant thymidine kinase(s) has an increase in helicity in the above region compared to wild-type thymidine kinase. If there is an increase in in helicity, it is expected that mutant thymidine kinase has an increased activity compared to wild-type thymidine kinase. The increase in activity can be confirmed by appropriate assays e.g. biochemical assays as provided and disclosed herein. The mutant tymidine kinases provided and to be used herein show an increase in helicity of >1 compared to wild-type tymidine kinase (e.g. an increase of at least (or 2) 1.03 and/or up to (or 5) 1.35, e.g. at least (or z) 1.10, 1.15, 1.16, 1.18, 1.20, 1.22, 1.23, 1.25, 1.30 or up to (or 5) 1.35, or even more than (or 2) 1.35, e.g. up to (or 2) 1.4, 1.5, 1.6, 1.7, 1.8, 1.9 or 2.0 or more). The “helicity” can be determined by assays described herein. Preferably, the helicity is determined in a region as defined herein below (the “helical domain”), corresponding to positions 70 to 110 of wild-type thymidine kinase.

The helical domain of tymidine kinase (approx. positions corresponding to positions 70 to 110 of wild-type thymidine kinase) is believed to play an important role in di-/tetramerisation of the protein and thus in activity of the protein. It is envisaged that the mutant tymidine kinase can be/can form a dimer or tetramer. For example, the therapeutically effective form may be a dimer or tetramer. In this context, the herein provided proteins e.g. as shown in SEQ ID NOs: 4, 6, 8, 10, 12, 14, 16, 18, 20, 22, 24, or 26 could be considered “monomers” that may form dimers or tetramers. In accordance with the above, a mutant thymidine kinase provided and to be used herein can comprise one or more amino acid substitutions at positions corresponding to positions 70 to 110 of wild-type thymidine kinase, specifically corresponding to positions 71, 72, 73, 74, 75 or 76 to positions 110, 109, 108, 107, 106, 105, 104, 103, 102, 101 or 100. For example, a mutant thymidine kinase provided and to be used herein can comprise one or more amino acid substitutions at positions corresponding to positions 70 to 100 of wild-type thymidine kinase.

In accordance with the above, a mutant thymidine kinase provided and to be used herein can comprise one or more amino acid substitutions at positions corresponding to positions 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, and/or 110. More specifically, the mutant thymidine kinase can comprise one or more amino acid substitutions at positions corresponding to positions 71 to 95 of wild-type thymidine kinase, e.g. one or more amino acid substitutions at positions corresponding to positions 71, 72, 73, 74, 75, 76, 77, 78, 79, 80, 81, 82, 83, 84, 85, 86, 87, 88, 89, 90, 91, 92, 93, 94, and/or 95. More preferably, the mutant thymidine kinase can comprise one or more amino acid substitutions at positions corresponding to one or more positions 73, 75, 83, 84, 90 and 95 of wild-type thymidine kinase, for example one or two amino acid substitutions at positions corresponding to positions 84 and/or 90 of wild-type thymidine kinase.

For example, the mutant thymidine kinase can comprise one or more aliphatic, nonpolar, and neutral amino acid, one or more acid/amide, acidic polar and negatively charged amino acid and/or one or more acid/amide, polar and neutral amino acid at one or more positions corresponding to positions 70 to 100 of wild-type thymidine kinase, e.g. one or more aliphatic, nonpolar, and neutral amino acid at one or more positions corresponding to positions 73, 75, 83, 90, and 95 of wild-type thymidine kinase.

The mutant thymidine kinase can comprise one or more aliphatic, nonpolar, and neutral amino acid at one or more positions corresponding to positions 73, 83, 90, and 95 of wild-type thymidine kinase. The aliphatic, nonpolar, and neutral amino acid may be alanine (Ala).

The thymidine kinase can comprise one or more aliphatic, nonpolar, and neutral amino acid at a position corresponding to position 75 of wild-type thymidine kinase. The aliphatic, nonpolar, and neutral amino acid may be glycine (Gly).

The mutant thymidine kinase can comprise one or more aliphatic, nonpolar, and neutral amino acid at a position corresponding to position 90 of wild-type thymidine kinase. The aliphatic, nonpolar, and neutral amino acid may be valine (Val).

The mutant thymidine kinase can comprise one or more acid/amide, acidic polar and negatively charged amino acid at one or more positions corresponding to positions 75, 84 and 90 of wild-type thymidine kinase. The acid/amide, acidic polar and negatively charged amino acid may be aspartic acid (Asp, D).

The mutant thymidine kinase can comprise one or two acid/amide, acidic polar and negatively charged amino acid at one or two positions corresponding to positions 84 and 90 of wild-type thymidine kinase. The acid/amide, acidic polar and negatively charged amino acid may be glutamic acid (Glu, E).

The mutant thymidine kinase can comprise an acid/amide, polar and neutral amino acid at a position corresponding to position 75 of wild-type thymidine kinase. The acid/amide, acidic polar and negatively charged amino acid may be glutamine (Gln, Q).

The mutant thymidine kinase provided and to be used herein can be mutant thymidine kinase,
a) wherein said mutant thymidine kinase comprises an aliphatic, nonpolar, neutral amino acid at a position corresponding to position 73 of wild-type thymidine kinase;
b) wherein said mutant thymidine kinase comprises an aliphatic, nonpolar, neutral amino acid or an acid/amide, polar, neutral amino acid or an acid/amide, acidic polar, negatively charged amino acid at a position corresponding to position 75 of wild-type thymidine kinase;
c) wherein said mutant thymidine kinase comprises an aliphatic, nonpolar, neutral amino acid at a position corresponding to position 83 of wild-type thymidine kinase;
d) wherein said mutant thymidine kinase comprises an acid/amide, acidic polar, negatively charged amino acid at a position corresponding to position 84 of wild-type thymidine kinase;
e) wherein said mutant thymidine kinase comprises an aliphatic, nonpolar, neutral amino acid or an acid/amide, acidic polar, negatively charged amino acid at a position corresponding to position 90 of wild-type thymidine kinase; and/or
f) wherein said mutant thymidine kinase comprises an aliphatic, nonpolar, neutral amino acid at a position corresponding to position 95 of wild-type thymidine kinase.

The mutant thymidine kinase provided and to be used herein can be a mutant thymidine kinase, wherein said mutant thymidine kinase can comprise
a) aliphatic, nonpolar, and neutral amino acids at positions corresponding to positions 73, 83, 90 and 95 of wild-type thymidine kinase and an acid/amide, acidic polar and negatively charged amino acid at a position corresponding to position 75 of wild-type thymidine kinase;
b) aliphatic, nonpolar, and neutral amino acids at positions corresponding to positions 73, 83, and 90 of wild-type thymidine kinase and an acid/amide, acidic polar and negatively charged amino acid at a position corresponding to position 75 of wild-type thymidine kinase;
c) an acid/amide, acidic polar and negatively charged amino acid at a position corresponding to position 84 of wild-type thymidine kinase and an aliphatic, nonpolar, and neutral amino acid at a position corresponding to position 90 of wild-type thymidine kinase;
d) aliphatic, nonpolar, and neutral amino acids at positions corresponding to positions 73, 90 and 95 of wild-type thymidine kinase and an acid/amide, acidic polar and negatively charged amino acid at a position corresponding to position 75 of wild-type thymidine kinase;
e) aliphatic, nonpolar, and neutral amino acids at positions corresponding to positions 73 and 90 of wild-type thymidine kinase and an acid/amide, acidic polar and negatively charged amino acid at a position corresponding to position 75 of wild-type thymidine kinase;
f) an aliphatic, nonpolar, and neutral amino acid at a position corresponding to position 90 of wild-type thymidine kinase;
g) acid/amide, acidic polar and negatively charged amino acids at positions corresponding to positions 75 and 90 of wild-type thymidine kinase;
h) an acid/amide, acidic polar and negatively charged amino acid at a position corresponding to position 84 of wild-type thymidine kinase and an aliphatic, nonpolar, and neutral amino acid at position corresponding to position 90 of wild-type thymidine kinase;
i) aliphatic, nonpolar, and neutral amino acids at positions corresponding to positions 75 and 90 of wild-type thymidine kinase and an acid/amide, acidic polar and negatively charged amino acid at a position corresponding to position 84 of wild-type thymidine kinase;
j) an acid/amide, acidic polar and negatively charged amino acid at a position corresponding to position 90 of wild-type thymidine kinase;
k) an aliphatic, nonpolar, and neutral amino acid at a position corresponding to position 90 of wild-type thymidine kinase; or
l) an acid/amide, polar and neutral amino acid at a position corresponding to position 75 of wild-type thymidine kinase and an acid/amide, acidic polar and negatively charged amino acid at a position corresponding to position 84 of wild-type thymidine kinase.

The mutant thymidine kinase provided and to be used herein can be a mutant thymidine kinase, wherein said mutant thymidine kinase can comprise
a) an aliphatic, nonpolar, and neutral amino acid at a position corresponding to position 90 of wild-type thymidine kinase; or
b) an acid/amide, acidic polar and negatively charged amino acid at a position corresponding to position 84 of wild-type thymidine kinase and an aliphatic, nonpolar, and neutral amino acid at a position corresponding to position 90 of wild-type thymidine kinase.

The mutant thymidine kinase provided and to be used herein can be a mutant thymidine kinase, comprising one or more aliphatic, nonpolar, and neutral amino acid(s), which may be one or more of alanine, glycine, valine, isoleucine, leucine and methionine. Preferably, the aliphatic, nonpolar, and neutral amino acid is one or more of alanine, glycine or valine.

The mutant thymidine kinase provided and to be used herein can be a mutant thymidine kinase comprising one or more acid/amide, acidic polar and negatively charged amino acid(s).

Preferably, the acid/amide, acidic polar and negatively charged amino acid is one or more of aspartic acid and glutamic acid.

The mutant thymidine kinase provided and to be used herein can be a mutant thymidine kinase comprising one or more acid/amide, polar and neutral amino acid is one or more of glutamine, asparagine, histidine, serine, threonine, tyrosine, and cysteine. Preferably, the acid/amide, polar and neutral amino acid is glutamine.

The mutant thymidine kinase provided and to be used herein can be a mutant thymidine kinase, wherein said mutant thymidine kinase comprises
a) alanine at positions corresponding to positions 73, 83, 90 and 95 of wild-type thymidine kinase and aspartic acid at a position corresponding to position 75 of wild-type thymidine kinase;
b) alanine at positions corresponding to positions 73, 83, and 90 of wild-type thymidine kinase and aspartic acid at a position corresponding to position 75 of wild-type thymidine kinase;
c) glutamic acid at a position corresponding to position 84 of wild-type thymidine kinase and alanine at a position corresponding to position 90 of wild-type thymidine kinase;
d) alanine at positions corresponding to positions 73, 90 and 95 of wild-type thymidine kinase and aspartic acid at a position corresponding to position 75 of wild-type thymidine kinase;
e) alanine at positions corresponding to positions 73, and 90 of wild-type thymidine kinase and aspartic acid at a position corresponding to position 75 of wild-type thymidine kinase;
f) alanine at a position corresponding to position 90 of wild-type thymidine kinase;
g) aspartic acid at a position corresponding to position 75 of wild-type thymidine kinase and glutamic acid at a position corresponding to position 90 of wild-type thymidine kinase;
h) aspartic acid at a position corresponding to position 84 of wild-type thymidine kinase and alanine at position corresponding to position 90 of wild-type thymidine kinase;
i) glycine at a position corresponding to position 75 of wild-type thymidine kinase, glutamic acid at a position corresponding to position 84 of wild-type thymidine kinase and alanine at a position corresponding to position 90 of wild-type thymidine kinase;
j) aspartic acid at a position corresponding to position 90 of wild-type thymidine kinase;
k) valine at a position corresponding to position 90 of wild-type thymidine kinase; or
l) glutamine at a position corresponding to position 75 of wild-type thymidine kinase and glutamic acid at a position corresponding to position 84 of wild-type thymidine kinase.

The mutant thymidine kinase provided and to be used herein can be a mutant thymidine kinase, wherein said mutant thymidine kinase comprises
a) alanine at a position corresponding to position 90 of wild-type thymidine kinase; or
b) aspartic acid amino acid at a position corresponding to position 84 of wild-type thymidine kinase and an alanine at a position corresponding to position 90 of wild-type thymidine kinase.

Specifically, herein provided are the mutant thymidine kinases, e.g. those as shown in Table 1.

Herein provided and to be used herein is a nucleic acid, wherein said nucleic acid is selected from the group consisting of:
a) a nucleic acid comprising a nucleotide sequence, wherein said nucleotide sequence encodes a mutant thymidine kinase, wherein said mutant thymidine kinase comprises an amino acid sequence as depicted in any one of SEQ ID NOs: 2, 4, 6, 8, 10, 12, 14, 16, 18, 20, 22 and 24;
b) a nucleic acid comprising a nucleotide sequence, wherein said nucleotide sequence encodes a mutant thymidine kinase, and wherein said nucleotide sequence is depicted in SEQ ID NO: 1, 3, 5, 7, 9, 11, 13, 15, 17, 19, 21 and 23;
c) a nucleic acid hybridizing under stringent conditions to the complementary strand of the nucleic acid as defined in (a) or (b) and wherein said nucleic acid comprises a nucleotide sequence wherein said nucleotide sequence encodes a mutant thymidine kinase;
d) a nucleic acid comprising a nucleotide sequence with at least 70% identity to the nucleotide sequence of the nucleic acids of any one of (a) to (c) and wherein said nucleotide sequence encodes a mutant thymidine kinase; and
e) a nucleic acid comprising a nucleotide sequence which is degenerate as a result of the genetic code to the nucleotide sequence of a nucleic acid of any one of (a) to (d) and wherein said nucleotide sequence encodes a mutant thymidine kinase.

Herein provided and to be used herein is a nucleic acid, wherein said nucleic acid is selected from the group consisting of:
a) a nucleic acid comprising a nucleotide sequence, wherein said nucleotide sequence encodes a mutant thymidine kinase, wherein said mutant thymidine kinase comprises an amino acid sequence as depicted in any one of SEQ ID NOs: 10 and 12;
b) a nucleic acid comprising a nucleotide sequence, wherein said nucleotide sequence encodes a mutant thymidine kinase, and wherein nucleotide sequence is depicted in SEQ ID NO: 9 and 11;
c) a nucleic acid hybridizing under stringent conditions to the complementary strand of the nucleic acid as defined in (a) or (b) and wherein said nucleic acid comprises a nucleotide sequence wherein said nucleotide sequence encodes a mutant thymidine kinase;
d) a nucleic acid comprising a nucleotide sequence with at least 70% identity to the nucleotide sequence of the nucleic acids of any one of (a) to (c) and wherein said nucleotide sequence encodes a mutant thymidine kinase; and
e) a nucleic acid comprising a nucleotide sequence which is degenerate as a result of the genetic code to the nucleotide sequence of a nucleic acid of any one of (a) to (d) and wherein said nucleotide sequence encodes a mutant thymidine kinase.

As used herein, and particularly in this context, the term “mutant thymidine kinase” refers to a “tymidine kinase with increased activity compared to a wild-type thymidine kinase and/or with increased helicity compared to a wild-type thymidine kinase”. Corresponding explanations, definitions and assays are provided herein.

It is understood that the mutant human thymidine kinase, particularly mutant human thymidine kinase 1, provided and to be used herein, comprises the mutation(s) as defined and explained herein, specifically one or more amino acid substitutions at positions corresponding to aa positions 70 to 100 of wild-type human thymidine kinase, particularly wild-type human thymidine kinase 1.

Apart from these mutations, it is envisaged herein that the sequence of the mutant human thymidine kinase at all other positions can comprise or consist of an amino acid sequence identical to that of its wild-type counterpart. For example, if the mutant human thymidine kinase is a mutant human thymidine kinase 1, it comprises the mutation(s) as defined and explained herein, and it can comprise or consist of an amino acid sequence outside of these mutations that is identical to that of wild-type human thymidine kinase 1.

This is exemplified by the sequences in the following table as also shown in SEQ ID NOs 33 to 44 (domain aa 71-95 of mutants shown in SEQ ID NOs 33 to 45 following the order of the table below). These sequences show mutations at specific positions corresponding to positions 71 to 95 of wild-type human thymidine kinase 1 while the amino acid sequence outside of these mutations at specific positions is that of wild-type human thymidine kinase 1:

| Tk1aa | | |
|---|---|---|
| 71 80 90 | | |
| \| \| \| | | |
| NTMEALPACLLRDVAQEALGVAVIG human wt Tk1 | helicity increase* | |
| (in-/decrease of spec, activity if mutated) | (fold of wt Tk1) | SEQ ID |
| NTAEDLPACLLRAVAQEALAVAVIA human TK1 mutM73AA75DD83AG9095A | 1.35 | 2 |
| NTAEDLPACLLRAVAQEALAVAVIG human TK1 mutM73AA75DD83AG90A | 1.30 | 4 |
| NTMEALPACLLRDEAQEALAVAVIG human TK1 mut V84EG90A | 1.23 | 6 |
| NTAEDLPACLLRDVAQEALAVAVIA human TK1 mutM73AA75DG9095A | 1.22 | 8 |
| NTAEDLPACLLRDVAQEALAVAVIG human TK1 mutM73AA75DC190A | 1.18 | 10 |
| NTMEALPACLLRDVAQEALAVAVIG human TK1 mut G90A | 1.16 | 12 |
| NTMEDLPACLLRDVAQEALEVAVIG human TK1 mut A75DG90E | 1.11 | 14 |
| NTMEALPACLLRDDAQEALAVAVIG human TK1 mut V84DG90A | 1.10 | 16 |
| NTMEGLPACLLRDEAQEALAVAVIG human TK1 mut A75GV84EG90A | 1.07 | 18 |
| NTMEALPACLLRDVAQEALDVAVIG human TK1 mut G90D | 1.05 | 20 |
| NTMEALPACLLRDVAQEALVVAVIG human TK1 mut G90V | 1.04 | 22 |
| NTMEQLPACLLRDEAQEALGVAVIG human TK1 mut A75QV84E | 1.03 | 24 |
| NTMEALPACFFRDVAQEALGVAVIG human TK1 mut L8081F | 0.77 | 26 |

Specifically, it is envisaged herein that the amino acid sequence of the mutant human thymidine kinase, particularly mutant human thymidine kinase 1, outside of the region corresponding to positions 70 to 100 of wild-type mutant human thymidine kinase (or in other terms before position 70 and after position 100), preferably corresponding to positions outside of positions 71 to 95 of wild-type human thymidine kinase (or in other terms before position 71 and after position 95), is identical to that of wild-type human thymidine kinase, particularly wild-type human thymidine kinase 1. For example, positions 1 to 69 and/or 96 to 234, preferably positions 1 to 70 and/or 96 to 234, of mutant human thymidine kinase, particularly mutant human thymidine kinase 1, can correspond/be identical to positions 1 to 69 and/or 101 to 234, preferably positions 1 to 70 and/or 96 to 234, of wild-type human thymidine kinase, particularly of wild-type human thymidine kinase 1, while positions 70 to 100, preferably 71 to 95, comprise/consist of an amino acid sequence with the mutations as described and explained herein (e.g. as shown in SEQ ID NOs 33 to 44). An exemplary amino acid sequence of wild-type human thymidine kinase 1 is shown in SEQ ID NO. 28.

It is understood that a “mutant human thymidine kinase” can without deferring from the gist of the invention comprise a lysine (Lys, K) at position 211 corresponding to position 211 of wild-type human thymidine kinase, e.g. as depicted herein below:

(SEQ ID NO: 31)
MSCINLPTVLPGSPSKTRGQIQVILGPMFSGKSTELMRRVRRFQIAQYKC
LVIKYAKDTRYSSSFCTHCRNTMEALPACLLRDVAQEALAVAVIGIDEGQ
FFPDIVEFCEAMANAGKTVIVAALDGTFQRKPFGAILNLVPLAESVVKLT
AVCMECFREAAYTKRLGTEKEVEVIGGADKYHSVCRLCYFKKASGQPAGP
DNKENCPVPGKPGEAVAARKLFAPQQILQWIQT

In the data base UniProt the entry describing the human cytosolic thymidine kinase (UniProtKB—P04183 (KITH_HUMAN)) (1. UniProtKB—P04183 (KITH_HUMAN). 2018; Available from: www at uniprot.org/uniprot/PO4183) lists the current knowledge about all the domains which are of importance regarding the structure/function relationship of the protein sequence. The amino acid position is located in none of the function related regions of the human TK1.

In other words, the amino acid “Arg, R” (e.g. in the sequence as depicted in any one of SEQ ID NOs: 2, 4, 6, 8, 10, 12, 14, 16, 18, 20, 22 and 24) at a position corresponding to position 211 of wild-type human tymidine kinase, preferably wild-type human tymidine kinase 1, can be replaced by the amino acid “Lys, K”. In nucleotide sequences the respective codon encoding “ARG, R” (e.g. codon at nt positions 841-843 in the sequences provided herein, or position 631-633 of SEQ ID NO. 30 in the appended sequence listing/FIG. 1), e.g. “AGG”, can be replaced by a codon encoding “Lys, K”, e.g. “AAG” or “AAA”.

The nucleotide sequences as shown in SEQ ID NO: 1, 3, 5, 7, 9, 11, 13, 15, 17, 19, 21, 23, 25, 27 and 30 in the description show the ORF (open reading frame), i.e. the coding sequence starting with the start codon “ATG”. It is of note that the numbering of positions of these herein provided or described nucleotide sequences as shown in SEQ ID NO: 1, 3, 5, 7, 9, 11, 13, 15, 17, 19, 21, 23, 25, 27 and 30 follows the numbering of the positions of the corresponding full-length transcript of wild-type thymidine kinase (SEQ ID NO: 29). For example, the start codon “ATG” at the indicated position 211-213 of SEQ ID NO: 1, 3, 5, 7, 9, 11, 13, 15, 17, 19, 21, 23, 25, 27 and 30 corresponds to position 211-213 of SEQ ID NO: 29.

In the appended sequence listing the nucleotide sequences as shown in SEQ ID NO: 1, 3, 5, 7, 9, 11, 13, 15, 17, 19, 21, 23, 25, 27 and 30 start with nt position 1. Thus, nt position 1 of the sequences in the appended sequence listing corresponds to nt position 211 of the nucleotide sequences as shown in SEQ ID NO: 1, 3, 5, 7, 9, 11, 13, 15, 17, 19, 21, 23, 25, 27 and 30 in the description, nt position 2 of the nucleotide sequences in the sequence listing corresponds to nt position 212 of the nucleotide sequences as shown in SEQ ID NO: 1, 3, 5, 7, 9, 11, 13, 15, 17, 19, 21, 23, 25, 27 and 30 in the description, and so on.

Thus, in one aspect, the nucleic acid provided and to be used herein is selected from the group consisting of:
a) a nucleic acid comprising a nucleotide sequence, wherein said nucleotide sequence encodes a mutant thymidine kinase, wherein said mutant thymidine kinase comprises an amino acid sequence as depicted in any one of SEQ ID NOs: 2, 4, 6, 8, 10, 12, 14, 16, 18, 20, 22 and 24 with the proviso that these amino acid sequences comprise a lysine (Lys, K) at a position corresponding to position 211 of wild-type tymidine kinase;
b) a nucleic acid comprising a nucleotide sequence, wherein said nucleotide sequence encodes a mutant thymidine kinase, and wherein said nucleotide sequence is depicted in SEQ ID NO: 1, 3, 5, 7, 9, 11, 13, 15, 17, 19, 21 and 23;
c) a nucleic acid hybridizing under stringent conditions to the complementary strand of the nucleic acid as defined in (a) or (b) and wherein said nucleic acid comprises a nucleotide sequence wherein said nucleotide sequence encodes a mutant thymidine kinase;
d) a nucleic acid comprising a nucleotide sequence with at least 70% identity to the nucleotide sequence of the nucleic acids of any one of (a) to (c) and wherein said nucleotide sequence encodes a mutant thymidine kinase; and
e) a nucleic acid comprising a nucleotide sequence which is degenerate as a result of the genetic code to the nucleotide sequence of a nucleic acid of any one of (a) to (d) and wherein said nucleotide sequence encodes a mutant thymidine kinase.

An exemplary nucleotide sequence encoding a mutant human tymidine kinase 1 with an Arg (R) at a position corresponding to position 211 of wild-type human tymidine kinase 1 is shown in SEQ ID NO: 30 (FIG. 1).

Furthermore, it is understood that a “mutant human thymidine kinase” can, without deferring from the gist of the invention, comprise the amino acids/contiguous amino acid stretch CSPAN (CysSerProAlaAsn; SEQ ID NO: 52) at positions corresponding to positions 230 to 234 of wild-type human thymidine kinase, preferably wild-type human thymidine kinase 1, and/or have the C-terminal amino acid sequence/amino acids/contiguous amino acid stretch CSPAN (CysSerProAlaAsn).

In other words, the amino acids/contiguous amino acid stretch “WIQT” (SEQ ID NO: 53) (e.g. in the sequence as depicted in any one of SEQ ID NOs: 2, 4, 6, 8, 10, 12, 14, 16, 18, 20, 22 and 24) at positions corresponding to positions 230 to 233 of wild-type human thymidine kinase, preferably wild-type human tymidine kinase 1, can be replaced by the amino acids/contiguous amino acid stretch CSPAN (at positions corresponding to positions 230 to 234 of wild-type human thymidine kinase, preferably wild-type human thymidine kinase 1).

Thus, in a related aspect, the nucleic acid provided and to be used herein is selected from the group consisting of:
a) a nucleic acid comprising a nucleotide sequence, wherein said nucleotide sequence encodes a mutant thymidine kinase, wherein said mutant thymidine kinase comprises an amino acid sequence as depicted in any one of SEQ ID NOs: 2, 4, 6, 8, 10, 12, 14, 16, 18, 20, 22 and 24 with the proviso that these amino acid sequences comprise a lysine (Lys, K) at a position corresponding to position 211 of wild-type tymidine kinase and/or comprise the amino acids/contiguous amino acid stretch CSPAN (CysSerProAlaAsn) at positions corresponding to positions 230 to 234 of wild-type human thymidine kinase, and/or have the C-terminal amino acid sequence/amino acids/contiguous amino acid stretch CSPAN (CysSerProAlaAsn);
b) a nucleic acid comprising a nucleotide sequence, wherein said nucleotide sequence encodes a mutant thymidine kinase, and wherein said nucleotide sequence is depicted in SEQ ID NO: 1, 3, 5, 7, 9, 11, 13, 15, 17, 19, 21 and 23 with the proviso that these nucleotide sequences encode the amino acids/contiguous amino acid stretch CSPAN (CysSerProAlaAsn) at positions corresponding to positions 230 to 234 of wild-type human thymidine kinase, and/or encode the C-terminal amino acid sequence/amino acids/contiguous amino acid stretch CSPAN (CysSerProAlaAsn) and/or comprise the nucleotide sequence tgc agc cct gcc aac tga (SEQ ID NO: 51) at nt positions corresponding to nt positions 898-915 of wild-type human thymidine kinase (e.g. as shown in SEQ ID NO. 29);
c) a nucleic acid hybridizing under stringent conditions to the complementary strand of the nucleic acid as defined in (a) or (b) and wherein said nucleic acid comprises a nucleotide sequence wherein said nucleotide sequence encodes a mutant thymidine kinase;
d) a nucleic acid comprising a nucleotide sequence with at least 70% identity to the nucleotide sequence of the nucleic acids of any one of (a) to (c) and wherein said nucleotide sequence encodes a mutant thymidine kinase; and
e) a nucleic acid comprising a nucleotide sequence which is degenerate as a result of the genetic code to the nucleotide sequence of a nucleic acid of any one of (a) to (d) and wherein said nucleotide sequence encodes a mutant thymidine kinase.

It is envisaged herein that the amino acid sequence of the mutant human thymidine kinase, particularly mutant human thymidine kinase 1, is—apart from the mutations described herein within the region corresponding to amino acid positions 70 to 100 of wild-type mutant human thymidine kinase—not necessarily identical to that of wild-type human thymidine kinase, particularly wild-type human thymidine kinase 1.

In one embodiment, the mutant human thymidine kinase, preferably mutant human thymidine kinase 1 comprises an arginine (Arg, R) at position 211 corresponding to position 211 of wild-type human thymidine kinase, preferably position 211 of wild-type human thymidine kinase 1.

Exemplary mutant thymidine kinases comprising an arginine (Arg, R) at position 211 comprise an amino acid sequence as depicted in any one of SEQ ID NOs: 2, 4, 6, 8, 10, 12, 14, 16, 18, 20, 22 and 24.

Thus, in one aspect, the nucleic acid provided and to be used herein is selected from the group consisting of:
a) a nucleic acid comprising a nucleotide sequence, wherein said nucleotide sequence encodes a mutant thymidine kinase, wherein said mutant thymidine kinase comprises an amino acid sequence as depicted in any one of SEQ ID NOs: 2, 4, 6, 8, 10, 12, 14, 16, 18, 20, 22 and 24;
b) a nucleic acid comprising a nucleotide sequence, wherein said nucleotide sequence encodes a mutant thymidine kinase, and wherein said nucleotide sequence is depicted in SEQ ID NO: 1, 3, 5, 7, 9, 11, 13, 15, 17, 19, 21 and 23 with the proviso that these nucleotide sequences encode an arginine at a position corresponding to position 211 of wild-type tymidine kinase;
c) a nucleic acid hybridizing under stringent conditions to the complementary strand of the nucleic acid as defined in (a) or (b) and wherein said nucleic acid comprises a nucleotide sequence wherein said nucleotide sequence encodes a mutant thymidine kinase;
d) a nucleic acid comprising a nucleotide sequence with at least 70% identity to the nucleotide sequence of the nucleic acids of any one of (a) to (c) and wherein said nucleotide sequence encodes a mutant thymidine kinase; and
e) a nucleic acid comprising a nucleotide sequence which is degenerate as a result of the genetic code to the nucleotide sequence of a nucleic acid of any one of (a) to (d) and wherein said nucleotide sequence encodes a mutant thymidine kinase.

Furthermore, it is understood that a “mutant human thymidine kinase” can without deferring from the gist of the invention comprise the amino acids/contiguous amino acid stretch WIQT (TrpIleGlnThr) at positions corresponding to positions 230 to 233 of wild-type human thymidine kinase, preferably wild-type human thymidine kinase 1, and/or have the amino acid (e.g. N, Asn) at a position corresponding to position 234 of wild-type human thymidine kinase, preferably wild-type human thymidine kinase 1, absent or deleted, e.g. as shown in SEQ ID NO: 12 or as depicted herein below:

(SEQ ID NO: 31)
MSCINLPTVLPGSPSKTRGQIQVILGPMFSGKSTELMRRVRRFQIAQYKC
LVIKYAKDTRYSSSFCTHDRNTMEALPACLLRDVAQEALAVAVIGIDEGQ
FFPDIVEFCEAMANAGKTVIVAALDGTFQRKPFGAILNLVPLAESVVKLT
AVCMECFREAAYTKRLGTEKEVEVIGGADKYHSCVRLCYFKKASGQPAGP
DNKENCPVPGKPGEAVAARKLFAPQQILQWIQT

Thus, in one embodiment, the mutant human thymidine kinase comprises the amino acids/contiguous amino acid stretch WIQT (TrpIleGlnThr) at positions corresponding to positions 230 to 233 of wild-type human thymidine kinase, preferably wild-type human thymidine kinase 1, and/or has the amino acid (e.g. N, Asn) at a position corresponding to position 234 of wild-type human thymidine kinase, preferably wild-type human thymidine kinase 1, absent or deleted. In a specific embodiment, the mutant human thymidine kinase has the N-terminal amino acid sequence/amino acids/contiguous amino acid stretch WIQT.

Exemplary mutant thymidine kinases comprising the amino acids/contiguous amino acid stretch WIQT (TrpIleGlnThr) at positions corresponding to positions 230 to 233 of wild-type human thymidine kinase and/or comprising the N-terminal amino acid sequence WIQT, are depicted in any one of SEQ ID NOs: 2, 4, 6, 8, 10, 12, 14, 16, 18, 20, 22 and 24 and/or are encoded by a nucleotide sequence as depicted in SEQ ID NO: 1, 3, 5, 7, 9, 11, 13, 15, 17, 19, 21 and 23.

Generally, in addition to the mutations in the region corresponding to positions 70 to 100 of wild-type mutant human thymidine kinase as defined herein, the amino acid sequence of the mutant human thymidine kinase can have further modifications, e.g. substitutions, additions and/or deletions of one or more amino acid(s), preferably of 1, 2, 3, 4, 5, 6, 7, 8, 9 or 10 or more amino acid(s), like 11, 12, 13, 14, 15, 16, 17, 18, 19 or 20 amino acids. For example, the amino acid sequence of the mutant human thymidine kinase can have further such modifications at positions corresponding to positions 1 to 69 and/or 101 to 234, preferably positions 1 to 70 and/or 96 to 234, of wild-type human thymidine kinase, particularly of wild-type human thymidine kinase 1.

Particularly such modifications are envisaged that do not affect the activity of the mutant human thymidine kinase as defined herein or that do not substantially affect the activity of the mutant human thymidine kinase as defined herein. “Not substantially affect” may mean in this context a decrease of the activity of the mutant human thymidine kinase of up to 10% compared to the mutant human thymidine kinase not carrying/having such modifications. Such modifications may confer advantageous properties, e.g. an increased stability of the mutant human thymidine kinase.

In accordance with the above, a mutant thymidine kinase comprising e.g. an amino acid sequence as depicted in any one of SEQ ID NOs: 2, 4, 6, 8, 10, 12, 14, 16, 18, 20, 22 and 24 comprises the mutations described herein and/or in the region corresponding to positions 70 to 100 of wild-type mutant human thymidine kinase, but its amino acid sequence can show—apart from the mutations described herein within the region corresponding to positions 70 to 100 of wild-type mutant human thymidine kinase—a certain variation, for example the modifications described above. This is meant and implied by the language “at least 70% identity” or “hybridizing under stringent conditions to the complementary strand of the nucleic acid” and the like as used herein.

For example, a variant of a mutant thymidine kinase comprises the following mutations:
a) alanine at positions corresponding to positions 73, 83, 90 and 95 of wild-type thymidine kinase and aspartic acid at a position corresponding to position 75 of wild-type thymidine kinase;
b) alanine at positions corresponding to positions 73, 83, and 90 of wild-type thymidine kinase and aspartic acid at a position corresponding to position 75 of wild-type thymidine kinase;
c) glutamic acid at a position corresponding to position 84 of wild-type thymidine kinase and alanine at a position corresponding to position 90 of wild-type thymidine kinase;
d) alanine at positions corresponding to positions 73, 90 and 95 of wild-type thymidine kinase and aspartic acid at a position corresponding to position 75 of wild-type thymidine kinase;
e) alanine at positions corresponding to positions 73, and 90 of wild-type thymidine kinase and aspartic acid at a position corresponding to position 75 of wild-type thymidine kinase;
f) alanine at a position corresponding to position 90 of wild-type thymidine kinase;
g) aspartic acid at a position corresponding to position 75 of wild-type thymidine kinase and glutamic acid at a position corresponding to position 90 of wild-type thymidine kinase;
h) aspartic acid at a position corresponding to position 84 of wild-type thymidine kinase and alanine at position corresponding to position 90 of wild-type thymidine kinase;
i) glycine at a position corresponding to position 75 of wild-type thymidine kinase, glutamic acid at a position corresponding to position 84 of wild-type thymidine kinase and alanine at a position corresponding to position 90 of wild-type thymidine kinase;
j) aspartic acid t a position corresponding to position 90 of wild-type thymidine kinase;
k) valine at a position corresponding to position 90 of wild-type thymidine kinase; or
l) glutamine at a position corresponding to position 75 of wild-type thymidine kinase and glutamic acid at a position corresponding to position 84 of wild-type thymidine kinase, respectively.

(Items a) to l) follow the order of the respective full-length amino acid sequence as shown in SEQ ID NOs: 2, 4, 6, 8, 10, 12, 14, 16, 18, 20, 22 and 24).

Accordingly, a variant of a mutant thymidine kinase comprising any of the above mutations, can show a certain variation in its amino acid sequence, for example further modifications as described above (e.g. substitutions, additions and/or deletions of one or more amino acid(s)).

For example, a variant of a mutant thymidine kinase comprising any of the above mutations can show a certain variation in its amino acid sequence, e.g. substitutions, additions and/or deletions of one or more amino acid(s) in an amino acid sequence as depicted in any one of SEQ ID NOs: 2, 4, 6, 8, 10, 12, 14, 16, 18, 20, 22 and 24.

The above explanations apply mutatis mutandis to mutant thymidine kinase protein(s) (i.e. proteins encoded by the herein provided nucleic acids) as provided and to be used herein, e.g. for use in treating cancer.

Accordingly, in one aspect, the mutant thymidine kinase provided and to be used herein is selected from the group consisting of:
a) a mutant thymidine kinase comprising an amino acid sequence as depicted in any one of SEQ ID NOs: 2, 4, 6, 8, 10, 12, 14, 16, 18, 20, 22 and 24;
b) a mutant thymidine kinase comprising an amino acid sequence encoded by a nucleic acid comprising a nucleotide sequence as depicted in SEQ ID NO: 1, 3, 5, 7, 9, 11, 13, 15, 17, 19, 21 and 23 with the proviso that these nucleotide sequences encode an arginine at a position corresponding to position 211 of wild-type tymidine kinase;
(c) a mutant thymidine kinase comprising an amino acid sequence encoded by a nucleic acid hybridizing under stringent conditions to the complementary strand of nucleic acids as defined in (b);
(d) a mutant thymidine kinase having at least 70% identity to the mutant thymidine kinase of any one of (a) to (c); and
(e) a mutant thymidine kinase comprising an amino acid sequence encoded by a nucleic acid being degenerate as a result of the genetic code to the nucleotide sequence of a nucleic acid as defined in (b) or (c).

In one aspect, the mutant thymidine kinase provided and to be used herein is selected from the group consisting of:
a) a mutant thymidine kinase comprising an amino acid sequence as depicted in any one of SEQ ID NOs: 2, 4, 6, 8, 10, 12, 14, 16, 18, 20, 22 and 24 with the proviso that these amino acid sequences comprise a lysine (Lys, K) at a position corresponding to position 211 of wild-type tymidine kinase and/or comprise the amino acids/contiguous amino acid stretch CSPAN (CysSerProAlaAsn) at positions corresponding to positions 230 to 234 of wild-type human thymidine kinase, and/or have the C-terminal amino acid sequence/amino acids/contiguous amino acid stretch CSPAN (CysSerProAlaAsn);
b) a mutant thymidine kinase comprising an amino acid sequence encoded by a nucleic acid comprising a nucleotide sequence as depicted in SEQ ID NO: 1, 3, 5, 7, 9, 11, 13, 15, 17, 19, 21 and 23 with the proviso that these nucleotide sequences encode the amino acids/contiguous amino acid stretch CSPAN (CysSerProAlaAsn) at positions corresponding to positions 230 to 234 of wild-type human thymidine kinase, and/or encode the C-terminal amino acid sequence/amino acids/contiguous amino acid stretch CSPAN (CysSerProAlaAsn);
(c) a mutant thymidine kinase comprising an amino acid sequence encoded by a nucleic acid hybridizing under stringent conditions to the complementary strand of nucleic acids as defined in (b);
(d) a mutant thymidine kinase having at least 70% identity to the mutant thymidine kinase of any one of (a) to (c); and
(e) a mutant thymidine kinase comprising an amino acid sequence encoded by a nucleic acid being degenerate as a result of the genetic code to the nucleotide sequence of a nucleic acid as defined in (b) or (c).

Also provided and to be used herein is/are (a) vector(s) comprising the nucleic acid(s), e.g. a gene therapy vector. Particularly, the vector may be an AAV vector, adenovirus vector, or a lentivirus vector.

Also provided and to be used herein are proteins of mutant thymidine kinase(s), e.g. protein encoded by the herein above defined nucleic acids.

Further, provided and to be used herein are composition(s) comprising the herein disclosed nucleic acid(s), vector(s), and/or protein(s). The composition may be a pharmaceutical composition.

Further, it is contemplated herein that the nucleic acid, vector, protein, and/or composition as provided and defined herein above is for use as a medicament. It is contemplated herein that the nucleic acid, vector, protein, and/or composition as provided and defined herein above is for use in therapy. Further, the use of the nucleic acid, vector, protein, and/or composition as provided and defined herein for the preparation of a pharmaceutical composition for use in therapy is envisaged.

Specifically, the nucleic acid, vector, protein, and/or composition as provided and defined herein may be used in treating cancer/in the treatment of cancer.

In a preferred aspect, the treatment of cancer comprises the administration of deoxythymidine.

According to our own results and published cell culture experiments, dTh levels exceeding 4 mM administered for prolonged times exert cytotoxic impacts on normal and neoplastic cells in addition to the cytostatic cell cycle arrest. [45] In trials where patients with relapsed leukemia or lymphoma with marrow and blood involvement were treated by infusion of dTh, dosages up to 240 g/sq m/day were applied for 14 to 29 days. Due to this administration regimen, mean plasma dTh levels of 5.5 mM were generated. [31, 46]. Side effects including nausea, vomiting, hepatotoxicity, electrolyte imbalances, mild central nervous system toxicity and bone marrow aplasia occurred. Although the therapy was able to induce a complete remission in some patients with acute leukemia previously refractory to treatment, the very large drug quantities, fluid volumes, and the prolonged therapy turned out to be impractical.

With the involvement of a mutant tymidine kinase (e.g. superactive Tk1), the required dTh concentrations can be reduced by a factor of 50 compared to concentrations necessary for wild-type Tk1s (5-20 mM dTh mean level necessary for wild-type tymidine kinase), resulting in mean plasma levels down to 0.1 mM dTh. This would reduce a necessary dosage down to a minimum of 5 g/sq m (square meter body surface)/day dTh e.g. administered by infusion (or even less). A dosage of 5 g dTh/sq m (square meter body surface)/day normally corresponds to an mean plasma level of about 0.1 mM dTh. All the observed and described adverse side effects would be heavily reduced or even eliminated. It can be expected that a therapy regimen employing these reduced concentration levels will be well tolerated by all patients, but still functioning.

Deoxythymidine (dTh) can be administered to a patient (particularly a human patient) herein in the treatment of cancer comprising the administration of deoxythymidine in amounts to achieve a mean plasma level or mean serum level in the patient of from (≥) 0.01 mM dTh and/or up to (≤) 5 mM dTh. For example, the mean plasma level or mean serum level of dTh herein can be of from (≥) 0.01 mM dTh and/or up to (≤) 5 mM dTh, e.g. of from (≥) 0.01 mM dTh, ≥0.02 mM dTh, ≥0.03 mM dTh or of from (≥) 0.04 mM dTh. and/or up to (≤) 0.05 mM dTh. ≤0.06 mM dTh, ≤0.07 mM dTh, ≤0.08 mM dTh. ≤0.09 mM dTh, ≤0.1 mM dTh, ≤0.2 mM dTh, ≤0.3 mM dTh, ≤0.4 mM dTh, or up to (≤) 0.5 mM dTh, or up to (≤) 0.6 mM dTh, ≤0.7 mM dTh, ≤0.8 mM dTh, ≤0.9 mM dTh, ≤1.0 mM dTh, ≤1.5 mM dTh, ≤2.0 mM dTh, ≤2.5 mM dTh, ≤3.0 mM dTh, ≤3.5 mM dTh, ≤4.0 mM dTh, ≤4.5 mM dTh, or ≤5.0 mM dTh. In one aspect, the For example, the mean plasma level or mean serum level of dTh herein can be of from (≥) 0.01 mM dTh and/or up to (≤5) mM dTh, preferably of from (≥) 0.01 mM dTh and/or up to (≤) 0.5 mM dTh.

In a preferred aspect, the mean plasma level or mean serum level is of from (≥) 0.01 mM dTh and/or up to (≤) 0.5 mM dTh. In a preferred aspect, the mean plasma level or mean serum level is of from (≥) 0.05 mM dTh and/or up to (≤) 0.5 mM dTh. In a preferred aspect, the mean plasma level or mean serum level is of from (≥) 0.01 mM dTh and/or up to (≤) 0.05 mM dTh. In a preferred aspect, the mean plasma level or mean serum level is of from (≥) 0.1 mM dTh and/or up to (≤) 0.5 mM dTh. Particularly for very low levels of dTh (e.g. of from about (≥) 0.01 mM dTh and/or up to about (≤) 0.05 mM dTh) mutant thymidine kinases with a high activity and/or high helicity can be used, e.g. with a helicity of ≥1.16, ≥1.18, ≥1.22, ≥1.23, ≥1.30, or ≥1.35.

Among “mean plasma level” and “mean serum level” herein the “mean serum level” is preferred. The “mean serum level” refers to the level of dTh (mM dTh) in serum (serum samples) of patients (particularly human patients) to be treated herein.

For example, by administering a dose/amount of 5 g dTh/sqm/day typically a mean plasma level or mean serum level of about 0.1 mM dTh can be achieved. Likewise, by administering a dose/amount of 25 g dTh/sqm/day typically a mean plasma level or mean serum level of about 0.5 mM dTh can be achieved. Thus, there is normally a linear correlation between the dose/amount of dTh administered and the mean plasma level or mean serum level.

Appropriate doses/amounts of dTh for administration to a patient to achieve a desired mean plasma level or mean serum level as defined above can be readily determined.

For a very low mean plasma level or mean serum level (e.g. of from (≥) 0.01 mM dTh and/or up to (≤) 0.05 mM dTh, the administration of dTh to the patient to treat cancer in accordance with the invention is normally not necessary. These very low levels correspond to physiologic (normal) dTh levels in (human) patients.

Mutant tymidine kinase as defined and provided herein can be used in monotherapy, e.g. in case the treatment does not comprise the administration of deoxythymidine (dTh).

The treatment of cancer may comprise administration of additional chemotherapeutic agents (e.g. Cytarabin (AraC) and/or 5-Fluoruracil (5-FU), and/or Trifluridine (trifluorothymidine or TFT)) and/or azidothymidine and/or aminothymidine and/or surgery and/or radiotherapy.

Indeed, cotherapy with deoxythymidine (dTh) and chemotherapeutic agents (e.g. Cytarabin (AraC) and/or 5-Fluoruracil (5-FU)) may be particularly advantageous in the herein provided cancer therapies for the following reasons. The following relates to synergistic effects of dTh and chemotherapeuticals.

It has been reported that the combination of deoxythymidine (dTh) with 5-fluorouracil (5-FU) increases the incorporation of 5-FU into RNA. This damage, leading to miscoding and inhibition of enzyme induction, is responsible for the cytotoxic effect. The explained therapeutic synergism has already been shown herein in murine mammary and colon tumors.

Three mechanisms are held responsible for the forced 5-FU uptake into the RNA: [47] a, inhibition of de-novo synthesis of uracil derivates by dTh nucleotides b, inhibition of nucleotide reductase by dTTP and thereby reducing the amount of the 5-FU metabolites and thus forcing more 5-FU into the RNA c, competition of thymine and pyrimidine-degrading enzymes in the liver thus decreasing clearance rates of 5-FU. A very important synergism of action between dTh and AraC has also been reported for lymphoma cell populations [48]. Concomitant dTh administration at a concentration of 1 mM also increases Cytarabin (AraC) incorporation into the DNA of rat hepatoma cells over 2-fold. The incorporation of AraC into DNA was increased 5-fold in L1210 leukemia cells after administration of 0.1 mM dTh. [26].

As illustrated herein, better outcomes using a combined dTh-superTK and chemotherapeutic approach can be achieved.

The cancer to be treated herein can be a solid cancer. In particular, the cancer may be cervical cancer, prostate carcinoma, ductal mammary carcinoma, melanoma, colon cancer, lung cancer, liver cancer, brain cancer (such as glioblastoma), or nerve cell cancer.

The following relates to vector systems and expression control.

The delivery of DNA or RNA into a cell in order to modulate gene expression for therapy, is limited by biological barriers such as the large size and the negative charge of the molecules. To overcome these barriers, various delivery systems have been created and even tested clinically in some cases. Although about 70% of the gene therapy clinical trials carried out so far used modified viruses as vectors, mostly due to the low delivery efficiency of synthetic vectors relative to viral ones, there are several limitations associated to viral delivery systems, such as carcinogenesis, immunogenicity, broad tissue tropism (which can be an advantage too), limited DNA packaging capacity and difficulty of vector production. Advantages of synthetic vectors are lower immunogenicity and the absence of pre-existing immunity shown against certain viruses in some patients [49]. Common to all kinds of regulatable vectors is that there has to be an efficient on-and-off switch of transgene expression, which should solely depend on an inducer drug, which is safe and well tolerated in humans. The main systems which have emerged from animal studies over the past years are the tetracycline and rapamycin-inducible systems, the mammalian steroid receptor (tamoxifen and mifepriston) and the insect steroid receptor (ecdysteroid) system. Of these four, the tetracycline inducible system is the most commonly studied and considered the most potent and clinically relevant [50]. Tetracycline inducible systems can be divided into Tet-On—Transcription is turned on in presence of tetracycline using the reverse Tet transactivator (rtTA) fusion Protein [51]- and Tet-Off—Transcription is turned off in presence of tetracycline using the tetracycline transactivator (tTA) protein [52]. Advantages of the Tet-On system are faster responsiveness and the fact that there is no need for continuous pharmacological treatment after gene expression termination. The disadvantage of the Tet-On system is possible expression leakiness in the absence of tetracycline.

The following relates to synthetic DNA/RNA vectors

In the systemic application of gene therapeutic vehicles, multiple barriers have to be overcome. Such vectors have to prevent degradation by serum endonucleases, and escape immune detection. Renal clearance from the blood has to be avoided as well as nonspecific interactions. The vectors have to be extravasated from blood stream to target tissues and mediate cell entry followed by endosomal escape. siRNA and miRNA must be loaded to the RISC, mRNA must bind to translation machinery and DNA has to be transported into the nucleus to affect gene expression. Lipid based DNA-vectors such as the cationic lipid DOTMA form small liposomes to encapsulate DNA. The cationic part associates with the DNA molecule while the hydrophobic tails form the surface of the liposome. Neutral phospholipids such as DOPE and cholesterol are used as “helper lipids” to enhance transfection and nanoparticle stability. Polymeric DNA vectors. Early examples are poly(L-lysine) (PLL) and polyethylenimine (PEI). PLL is a lysine homopolypeptide and its ability to condense DNA is known since the 1960s [53, 54]. However, PLL has poor transfection activity due to its mostly positively charged amino groups. Therefore, PEGylated PLL was tested to have potential for clinical use regarding its safety and tolerability. PEI has been reported to afford gene transfection into the lungs and into tumors of mouse models. Other polymers currently being evaluated are poly[(2-dimethylamino) ethyl methacrylate] (pDMAEMA), poly(f-amino ester)s and various carbohydrate-based polymers and dendrimers. An alternative to DNA-based gene therapy is the in vivo protein expression mediated by mRNA. Although it is less stable than DNA, advantages are reduced immunogenicity, no potential for mutagenesis due to missing genomic integration and that there is no requirement for nuclear localization. Some requirements instead, are the same as for DNA based vectors, such as extravasation, cell entry and protection against serum endonucleases. Synthetic siRNAs are structures that mimic the cleavage product of the enzyme Dicer. They are incorporated into the RNA interference (RNAi) machinery in the same way as endogenous small RNAs. siRNAs have the potential to silence nearly any targeted gene after introduction into cells. Challenges of in vivo siRNA delivery are similar to those of mRNA delivery. Strategies therefore are chemical modifications of the RNA components and encapsulation inside nanoparticles. Those nanoparticles can be lipid-based, like the stable nucleic acid-lipid particle (SNALP) formulation, which is currently under clinical evaluation. Other nanoparticles are polymer-based, such as the Cyclodextrin polymer (CDP)-based nanoparticles. Furthermore, delivery ligands which are attached to the siRNA cargo are promising delivery systems. DPCs and GalNAc conjugates are the most clinically advanced platforms and include several components, each with a particular function in the delivery process. In solid tumors, both Lipid-based nanoparticles (ALN-VSPO2, Alnylam Pharmaceuticals) and CDP-based nanoparticles (CALAA-01, Calando Pharmaceuticals) have been under clinical evaluation [49]. For our purposes, effectine transfection reagent (Quiagen) has proven to be a suitable DNA transfection vector substance. According to the producer it “is a nonliposomal lipid reagent for DNA transfection into a broad range of cell types. Due to its low cytotoxicity, Effectene Transfection Reagent is highly suitable for transfection of primary cells and many sensitive cell lines.” The following relates to viral vectors.

Several viral vector systems have been tested in vitro, in mouse models and even in clinical studies. Vaccinia Virus strains are mostly used in oncolytic therapy. Oncolytic viruses are designed to induce a tumor specific immunity while replicating specifically in cancer cells, leading to lysis of tumor cells [55]. Lentivirus-based retroviral delivery systems are able to perform RNA interference on different pathological conditions (e.g. resulting in activation of apoptosis), such as ovarian cancer [56], human melanoma cells [57] and even parasitic diseases like schistosomiasis [58]. Potential for medical applications has also been shown by the MLV (murine leukemia virus) derived retroviral vector. Migration of the MLV pre-integration complex into the nucleus of infected cells requires mitosis for nuclear membrane breakdown.

Since a majority of healthy human cells exist in a quiescent state in vivo (in contrast to strongly proliferating tumor cells), MLV vectors are thus promising vehicles for save cancer gene therapy. Problems with MLV vectors are unintended silencing or position effect variation of gene expression due to random integration into the host genome and affection of transgene expression by the flanking host chromatin [59]. The adenovirus (Ad) vector is the most commonly used vector for gene therapy due to its tightly regulated expression of transgenes. For investigations in the gene therapy field, the Tet-inducible expression control system is the most widely used, despite a certain expression leakiness. According to the Chen research group [60], this problem was solved by combining a modified, “second generation” rtTAs with co-expression of a tet-controlled transcriptional silencer. Chen et al. created the adenoviral vector called pAd5-KRAB-Bi-Tet-On.

The Adeno-associated Virus is very attractive alternative to the Adenovirus system, because it causes lesser immunological problems, especially after repeated administration in test animals.

Therefore helper virus free recombinant adeno-associated virus expression systems have been developed to serve as important tools for gene delivery [61]. As an alternative to the Ad-system we took advantage of the AAV Helper-Free System provided by Agilent Technologies [62] in order to establish the set up for successive animal experiments. The components are:
pAAV-MCS vector
pAAV-LacZ vector
pAAV-RC plasmid
pMCS-MCS-vector
pHelper plasmid

As illustrated in the appended examples, the cloning strategy, propagation and the use of the AAV-system can be easily performed, for example by inserting the superTk1 cDNA into the BamHI restriction site in the pAAV-MCS vector. By a triple transfection together with the pAAV-RC and pHelper plasmids into HEK-293 cells, high titer AAV virussuperTK1 particles were produced and isolated by centrifugation. The big advantage of the AAV system is in addition to the lesser immunological problems, that all target cells are infected independent of their state of growth (G0, G1, S, G2 or M-phase).

The meaning of the terms “polypeptide”, “protein”, “amino acid sequence”, “nucleic acid”, “nucleotide sequence” etc. is well known in the art and is used accordingly in context of the present invention. For example, “nucleic acid” as used herein refer(s) to all forms of naturally occurring or recombinantly generated types of nucleic acids as well as to chemically synthesized nucleic acids. This term also encompasses nucleic acid analogues and nucleic acid derivatives. The term “nucleic acid” can refer to deoxyribonucleic acid (DNA) and ribonucleic acid (RNA). The “nucleic acid” may be made by synthetic chemical methodology known in the art, or by the use of recombinant technology, or even by isolation from natural sources, or by a combination thereof. The DNA and RNA may optionally comprise unnatural nucleotides and may be single or double stranded. “Nucleic acid” also refers to sense and anti-sense DNA and RNA, that is, a nucleotide sequence which is complementary to a specific sequence of nucleotides in DNA and/or RNA. Furthermore, the term “nucleic acid” may refer to DNA or RNA or hybrids thereof or any modification thereof that is known in the state of the art (see, e.g., U.S. Pat. Nos. 5,525,711, 4,711,955, 5,792,608 or EP 302175 for examples of modifications). The nucleic acid molecule(s) may be single- or double-stranded, linear or circular, natural or synthetic, and without any size limitation. For instance, the nucleic acid molecule(s) may be genomic DNA, cDNA, mRNA, antisense RNA, or a DNA encoding such RNAs or chimeroplasts (Cole-Strauss (1996)[63]. Said nucleic acid molecule(s) may be in the form of a plasmid or of viral DNA or RNA. “Nucleic acid” may also refer to (an) oligonucleotide(s), wherein any of the state of the art modifications such as phosphothioates or peptide nucleic acids (PNA) are included.

(A) nucleotide sequence(s) with a certain level of identity to the herein provided sequences can be identified by the skilled person using methods known in the art, e.g. by using hybridization assays or by using alignments, either manually or by using computer programs such as those mentioned herein below in connection with the definition of the term “hybridization” and degrees of identity.

The nucleotide sequence may be at least 70% identical to the nucleotide sequence as provided herein, e.g. as shown in any one of SEQ ID NO. 1, 3, 5, 7, 9, 11, 13, 15, 17, 19, 21 or 23. More preferably, the nucleotide sequence is at least 90%, 91%, 92%, 93%, 94%, 95%, 96%, 97% or 98% identical to the nucleotide sequence as provided herein, e.g. as shown in any one of SEQ ID NO. 1, 3, 5, 7, 9, 11, 13, 15, 17, 19, 21 or 23, wherein the higher values are preferred. Most preferably, the nucleotide sequence is at least 99% identical to the nucleotide sequence as provided herein, e.g. as shown in any one of SEQ ID NO. 1, 3, 5, 7, 9, 11, 13, 15, 17, 19, 21 or 23, as shown in SEQ ID NO. 1.

Hybridization assays for the characterization of nucleic acids with a certain level of identity to the nucleotide sequence as provided herein are well known in the art; see e.g. Sambrook, Russell “Molecular Cloning, A Laboratory Manual” [64]; Ausubel, “Current Protocols in Molecular Biology” [65]. The term “hybridization” or “hybridizes” as used herein may relate to hybridizations under stringent or non-stringent conditions. If not further specified, the conditions are preferably non-stringent. Said hybridization conditions may be established according to conventional protocols described, e.g., in Sambrook (2001)[64]; Ausubel (1989)[65], or Higgins and Hames (1985.) “Nucleic acid hybridization, a practical approach [66]). The setting of conditions is well within the skill of the artisan and can be determined according to protocols described in the art. Thus, the detection of only specifically hybridizing sequences will usually require stringent hybridization and washing conditions such as, for example, the highly stringent hybridization conditions of 0.1×SSC, 0.1% SDS at 65° C. or 2×SSC, 60° C., 0.1% SDS. Low stringent hybridization conditions for the detection of homologous or not exactly complementary sequences may, for example, be set at 6×SSC, 1% SDS at 65° C. As is well known, the length of the probe and the composition of the nucleic acid to be determined constitute further parameters of the hybridization conditions. It is envisaged herein that a nucleic acid can be a primer or probe, for example, a nucleic acid hybridizing under stringent conditions to the complementary strand of the nucleic acid as provided herein (or of a fragment thereof as defined herein). Primers and probes are often in the range of 10-30 nucleotides.

In accordance with the above, the terms “homology” or “percent homology” or “identical” or “percent identity” or “percentage identity” or “sequence identity” in the context of two or more nucleic acid sequences refers to two or more sequences or subsequences that are the same, or that have a specified percentage of nucleotides that are the same (at least 70%, 75%, 80%, 85%, most preferably at least 90%, 91%, 92%, 93%, 94%, 95%, 96%, 97% or 98% identity, most preferably at least 99% identity), when compared and aligned for maximum correspondence over a window of comparison (preferably over the full length), or over a designated region as measured using a sequence comparison algorithm as known in the art, or by manual alignment and visual inspection. Sequences having, for example, 75% to 90% or greater sequence identity may be considered to be substantially identical. Such a definition also applies to the complement of a test sequence. Preferably the described identity exists over the full length. Those having skill in the art will know how to determine percent identity between/among sequences using, for example, algorithms such as those based on CLUSTALW computer program [67] or FASTDB [68], as known in the art.

Although the FASTDB algorithm typically does not consider internal non-matching deletions or additions in sequences, i.e., gaps, in its calculation, this can be corrected manually to avoid an overestimation of the % identity. CLUSTALW, however, does take sequence gaps into account in its identity calculations. Also available to those having skill in this art are the BLAST and BLAST 2.0 algorithms (Altschul, (1997)[69]; Altschul (1993) [70]; Altschul (1990) [71]. The BLASTN program for nucleic acid sequences uses as defaults a word length (W) of 11, an expectation (E) of 10, M=5, N=4, and a comparison of both strands. The BLOSUM62 scoring matrix (Henikoff (1989) [72] uses alignments (B) of 50, expectation (E) of 10, M=5, N=4, and a comparison of both strands.

In order to determine whether an nucleotide residue in a nucleic acid sequence corresponds to a certain position in the nucleotide sequence of e.g. SEQ ID NO. 1, 3, 5, 7, 9, 11, 13, 15, 17, 19, 21 or 23, respectively, the skilled person can use means and methods well-known in the art, e.g., alignments, either manually or by using computer programs such as those mentioned herein. For example, BLAST 2.0, which stands for Basic Local Alignment Search Tool BLAST [69-71], can be used to search for local sequence alignments. BLAST, as discussed above, produces alignments of nucleotide sequences to determine sequence similarity. Because of the local nature of the alignments, BLAST is especially useful in determining exact matches or in identifying similar sequences. The fundamental unit of BLAST algorithm output is the High-scoring Segment Pair (HSP). An HSP consists of two sequence fragments of arbitrary but equal lengths whose alignment is locally maximal and for which the alignment score meets or exceeds a threshold or cut-off score set by the user. The BLAST approach is to look for HSPs between a query sequence and a database sequence, to evaluate the statistical significance of any matches found, and to report only those matches which satisfy the user-selected threshold of significance. The parameter E establishes the statistically significant threshold for reporting database sequence matches. E is interpreted as the upper bound of the expected frequency of chance occurrence of an HSP (or set of HSPs) within the context of the entire database search. Any database sequence whose match satisfies E is reported in the program output.

Analogous computer techniques using BLAST [69-71] are used to search for identical or related molecules in nucleotide databases such as GenBank or EMBL. This analysis is much faster than multiple membrane-based hybridizations. In addition, the sensitivity of the computer search can be modified to determine whether any particular match is categorized as exact or similar. The basis of the search is the product score, which is defined as:

$$\frac{\% sequence identity \times \% maximum BLAST score}{100}$$

and it takes into account both the degree of similarity between two sequences and the length of the sequence match. For example, with a product score of 40, the match will be exact within a 1-2% error; and at 70, the match will be exact. Similar molecules are usually identified by selecting those, which show product scores between 15 and 40, although lower scores may identify related molecules. Another example for a program capable of generating sequence alignments is the CLUSTALW computer program [67] or FASTDB [68](Brutlag (1990), as known in the art.

The explanations and definitions given herein above in respect of “identity of nucleotide sequences” apply, mutatis mutandis, to “amino acid sequences” of the herein provided mutant thymidine kinases as depicted in SEQ ID NOs: 2, 4, 6, 8, 10, 12, 14, 16, 18, 20, 22 or 24, respectively, as explained below.

The protein to be used in accordance with the present invention may have at least 70% identity/similarity to the proteins having the amino acid sequence as, for example, depicted in SEQ ID NOs: 2, 4, 6, 8, 10, 12, 14, 16, 18, 20, 22 or 24, respectively. More preferably, the protein has at least 90%, 91%, 92%, 93%, 94%, 95%, 96%, 97% or 98% identity/similarity to the proteins depicted in SEQ ID NOs: 2, 4, 6, 8, 10, 12, 14, 16, 18, 20, 22 or 24, respectively, wherein the higher values are preferred. Most preferably, the polypeptide has at least 99% homology to the protein as depicted in SEQ ID NOs: 2, 4, 6, 8, 10, 12, 14, 16, 18, 20, 22 or 24, respectively.

The use of fragment(s) and/or derivative(s) of the herein provided mutant thymidine kinase (i.e. of mutant thymidine kinase proteins), is contemplated, provided that the fragment(s) and/or derivative(s) of the mutant thymidine kinase(s) have an increased activity and/or increased helicity compared to wild-type thymidine kinase. Likewise, the use of fragment(s) and/or derivative(s) of nucleic acids encoding such mutant thymidine kinase(s) is envisaged, provided that the encoded mutant thymidine kinase(s) have an increased activity and/or increased helicity compared to wild-type thymidine kinase. The terms “nucleic acid” and “mutant thymidine kinase (protein)” encompass such fragment(s) and/or derivative(s).

As used herein, the terms “comprising”, “including”, “having” or grammatical variants thereof are to be taken as specifying the stated features, integers, steps or components but do not preclude the addition of one or more additional features, integers, steps, components or groups thereof. The terms “comprising”/“including”/“having” encompass the terms “consisting of” and “consisting essentially of”. Thus, whenever the terms “comprising”/“including”/“having” are used herein, they can be replaced by “consisting essentially of” or, preferably, by “consisting of”.

The terms “comprising”/“including”/“having” mean that any further component (or likewise features, integers, steps and the like) can be present.

The term “consisting of” means that no further component (or likewise features, integers, steps and the like) can be present.

The term “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed product, composition, device or method and the like.

Thus, the term “consisting essentially of” means that specific further components (or likewise features, integers, steps and the like) can be present, namely those not materially affecting the essential characteristics of the product, composition, device or method. In other words, the term “consisting essentially of” (which can be interchangeably used herein with the term “comprising substantially”), allows the presence of other components in the product, composition, device or method in addition to the mandatory components (or likewise features, integers, steps and the like), provided that the essential characteristics of the product, composition, device or method are not materially affected by the presence of other components.

As used herein the term “about” refers to 10%.

The present invention is further described by reference to the following non-limiting figures and examples.

The following examples illustrate the invention:

The Figures show:

FIG. 1. Human superTk1 nt sequence that was inserted into the pUHDHyg10.3 The differences to the wt Tk1 sequences (NM_003258) are underlined and printed in bold; especially the point mutations at nts 269/270 counted from the start ATG results in a substitution of G>A is relevant for the increase in specific activity of the superTk1. All the other differences are consequences of the origin of the cDNA and/or due to subcloning conditions.

The human superTk1 nt sequence is depicted as SEQ ID NO: 30.

FIG. 2. Human superTk1 aa expressed from the recombinant system pUHDHyg10.3sequence

The differences to the wt Tk1 sequences (NP_003249) are underlined and printed in bold; especially the point mutation at aa 90 that causes a transition from G>A is relevant for the increase in specific activity of the superTk1. All the other changes are consequences of the origin of the cDNA and/or due to subcloning conditions. The human superTk1 aa sequence is depicted as SEQ ID NO: 12.

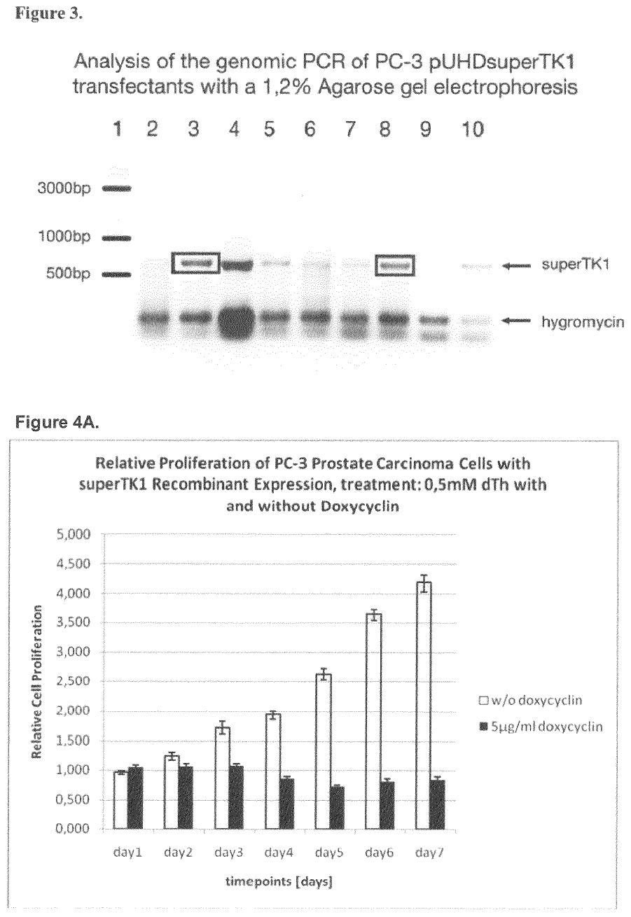

FIG. 3. Agarose gel analysis of genomic PCR-results of stable transfected clones with pUDHsuperTk1 The PC-3 pUHDsuperTK1 clone 2 and 13 were chosen for the growth experiments due to the strongest superTK1 bands detectable at the correct length of approx. 665 bp on a 1.2% Agarose gel (see red box).

Loading scheme of the 1.2% agarose gel:

| Lane | Sample | Description |
|---|---|---|
| 1 | 1 kb ladder (10 μl) | |
| 2 | PC-3 pUHDsupTK1 clone 1 | A very faint superTK1 band |
| | | was detectable |
| 3 | PG-3 pUHDsupTK1 clone 2 | A strong superTK1 band was |
| | | detectable |
| 4 | PC-3 pUHDsupTK1 clone 7 | All detectable bands are very |
| | | strong due to overloading of too |
| | | much DNA |
| 5 | PC-3 pUHDsupTK1 clone 10 | A faint superTK1 band was |
| | | detectable |
| 6 | PC-3 pUHDsupTK1 clone 11 | A faint superTK1 band was |
| | | detectable |
| 7 | PC-3 pUHDsupTK1 clone 12 | A faint superTK1 band was |
| | | detectable |
| 8 | PC-3 pUHDsupTK1 clone 13 | A strong superTK1 band was |
| | | detectable |
| 9 | Empty hygromycin vector | Only the hygromycin band was |
| | | detectable at 149 bp |
| 10 | Plasmid pUHDsuperTK1 #117 | A superTK1 band at 665 bp |
| | | and a hygromycin band at |
| | | 149 bp was detectable |

FIG. 4A. Stable transfectant clone 2 of primary prostate carcinoma cells (PC-3) with superTk1 carrying pUHDHyg10.3 and treatment with 0.5 mM dTh

The histogram shows the relative cell proliferation of PC-3 prostate carcinoma cells with stably integrated (into the genome) superTK1 gene (pUDHHyg driven), verified by a genomic PCR. The cytostatic treatment with 0.5 mM deoxythymidine (dTh) lasted for seven days, replenished every day. The bars show the mean values of cell numbers of sextuplicates, each sample measured twice; the error bars display the standard error of the means. Cell batches that expressed superTk1 received 5 μg/ml doxycyclin replenished on a daily basis. The control samples received all other constituents (dTh, antibiotics) but no doxycyclin. In addition, the cell culture medium was renewed completely every 72 hrs in all wells in order to avoid unwanted starvation or deprivation effects.

FIG. 4B. Statistical evaluation of the inhibition experiment presented in FIG. 4A

The PC-3 proliferation data recorded during inhibition by 0.5 mM dTh were subjected to a statistical analysis; Box and Whiskers plots are presented

FIG. 4C. Stable transfectant clone 7 of primary prostate carcinoma cells (PC-3) with wtTk1 carrying pUHDHyg10.3 and treatment with 0.5 mM dTh

The histogram shows the relative cell proliferation of PC-3 prostate carcinoma cells with stably integrated wtTK1 gene (pUDHHyg driven), verified by a genomic PCR. The cytostatic treatment with 0.5 mM deoxythymidine (dTh) lasted for five days, replenished every day. The bars show the mean values of cell numbers of triplicates, each sample measured twice; the error bars display the standard error of the means. Cell batches that expressed wtTk1 received 5 μg/ml doxycyclin replenished on a daily basis. The control samples received all other constituents (dTh, antibiotics) but no doxycyclin. In addition, the cell culture medium was renewed completely after 72 hrs in all wells in order to avoid unwanted starvation or deprivation effects.

FIG. 4D. DNA Profiles of PC-3 prostate cancer cells with super TK (clone 2) recombinant expression after dTh and doxycycline supply for 3 days DNA profiles of propidium iodide DNA labeled primary carcinoma cells: PC-3 pUHD_hygr superTK1 stable transfectants

a) and b) show curves acquired by the FACS Calibur a) untreated cells and b) depicts cells supplied with 0.5 mM dTh and 5 μg/ml doxycycline for three days. c) The bar graph shows which percentage of untreated cells (control) or those supplied with either substrate only (0.5 mM dTh) or doxycycline 5 μg/ml only (doxy), or both (0.5 dTh+doxy), is undergoing which cell cycle phase at the time point of ethanol fixation on day 3.

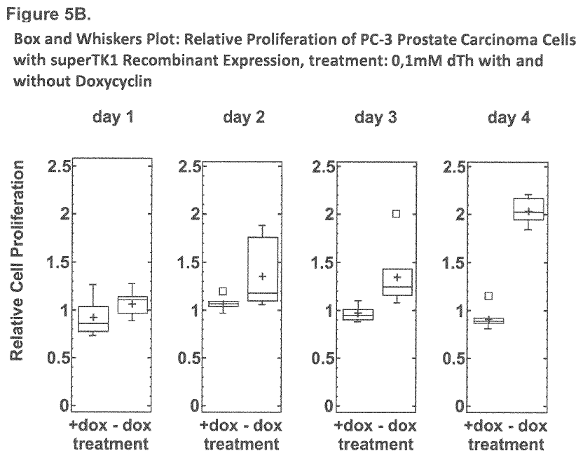

FIG. 5A. Stable transfectant clone 13 of primary prostate carcinoma cells (PC-3) with superTk1 carrying pUHDHy10.3 and treatment with 0.1 mM dTh

The histogram shows the relative cell proliferation of PC-3 prostate carcinoma cell clone 13 with stable integrated superTK1 gene (pUDHHygr driven), verified by genomic PCR. The cytostatic treatment with 0.1 mM deoxythymidine (dTh) lasted for four days, replenished every day. The bars show the mean values of cell numbers of triplicates, each sample measured twice; the error bars display the standard error of the means. Cell batches that expressed superTk1 received 5 μg/ml doxycyclin replenished on a daily basis. The control samples received all other constituents (dTh, antibiotics) but no doxycyclin. In addition, the cell culture medium was renewed completely every 72 hrs in all wells in order to avoid unwanted starvation or deprivation effects.

FIG. 5B. Statistical evaluation of the inhibition experiment presented in FIG. 5A

The PC-3 proliferation data recorded during inhibition by 0.1 mM dTh were subjected to a statistical analysis; Box and Whiskers Plots are presented. The Kruskal-Wallis test shows a P-value, which is less than 0.05 from day 3 on; this means that there is a statistically significant difference amongst the medians of the treated sample and its control at the 95.0% confidence level. The method used to discriminate among the means is Fisher's least significant difference (LSD) procedure

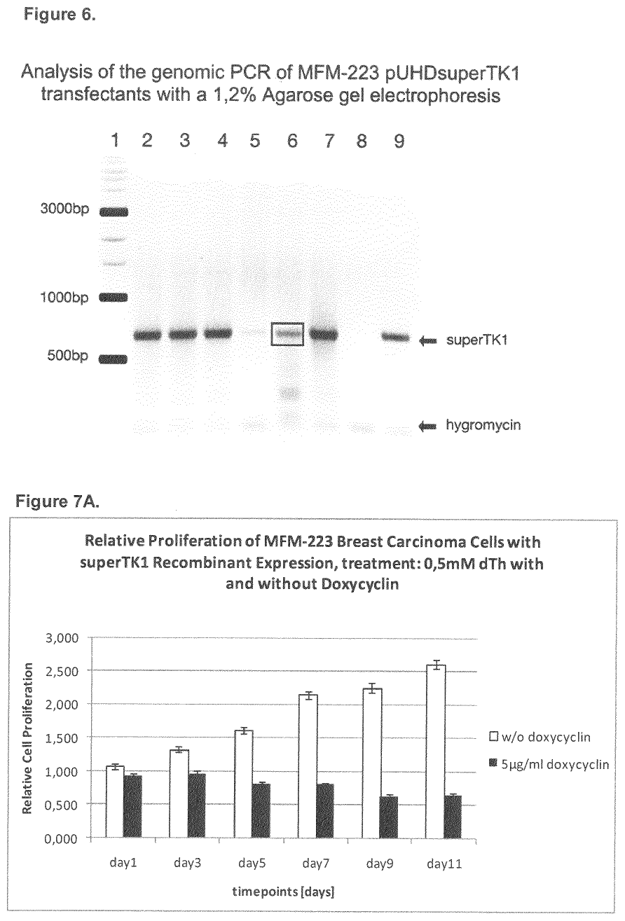

FIG. 6. Agarose gel analysis of genomic PCR-results of stable transfected clones with pUHDsuperTk1 in MFM-223 cells

The MFM-223 pUHDsuperTK1 clone 8 was chosen for the growth experiments because it was the fastest growing clone of this slowly growing cell line despite having only a weaker superTK1 band compared to others (like as clone 16 in lane 7) at the correct length of approx. 665 bp on a 1.2% arose gel (see red box).

Loading scheme of the 1.2% agarose gel:

| Lane | Sample | Description |
|---|---|---|
| 1 | 1 kb ladder (10 μl) | |
| 2 | MFM-223 pUHDsuperTK1 | A very strong superTK1 band |
| | clone 1 | was detectable |
| 3 | MFM-223 pUHDsuperTK1 | A very strong superTK1 band |
| | clone 7 | was detectable |
| 4 | MFM 223 pUHDsuperTK1 | A very strong superTK1 band |
| | clone 5 | was detectable |
| 5 | MFM-223 pUHDsuperTK1 | A very faint superTK1 band |
| | clone 15 | was detectable |
| 6 | MFM-223 pUHDsuperTK1 | A faint superTK1 band |
| | clone 8 | was detectable |
| 7 | MFM-223 pUHDsuperTK1 | A very strong superTK1 band |
| | clone 16 | was detectable |
| 8 | pUHDhygr_empty vector | Only the hygromycin band |
| | | was detectable at 149 bp |
| 9 | Plasmid pUHDsuperTK1 #117 | A superTK1 band at 665 bp |
| | | and a hygromycin band at |
| | | 149 bp was detectable |

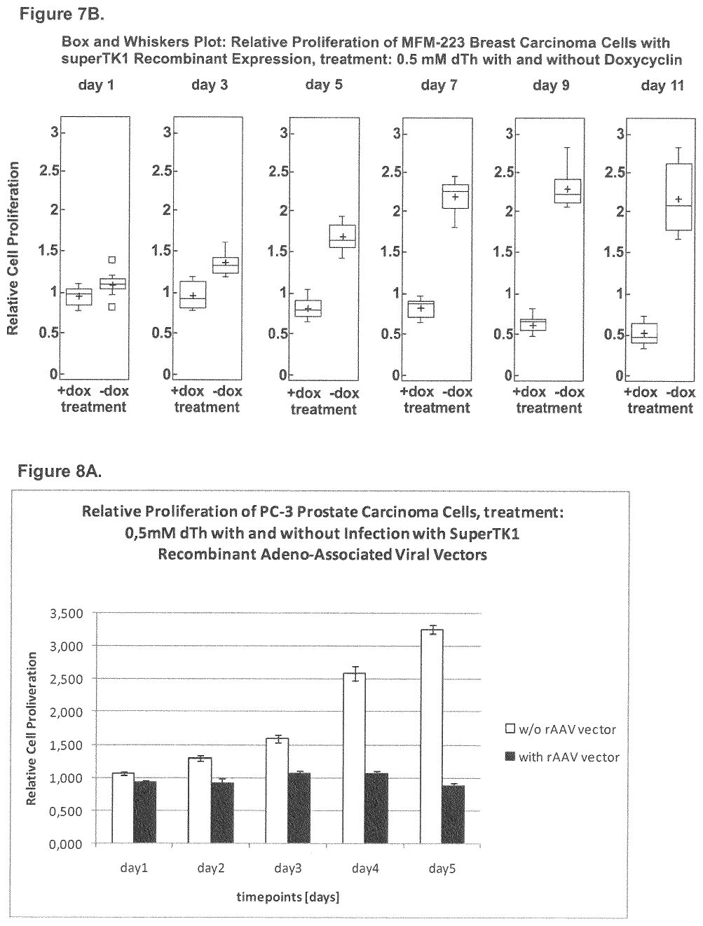

FIG. 7A. Stable transfectant clone 8 of primary MFM-223 Breast Carcinoma with superTk1 carrying pUHDHyg10.3 and treatment with 0.5 mM dTh

The histogram presented in FIG. 7A shows the relative cell proliferation of MFM-223 breast carcinoma cells with stable integrated pUDHhygr-driven superTK1 gene for a period of seven days. The presence of the superTk1 was verified by genomic PCR. The bars show the mean values of cell numbers of sixtuplicates, each sample measured twice, and the error bars display the standard error of the means. All samples were treated with 0.5 mM deoxythymidine (dTh) per day. To one half of the/wells 5 μg/ml doxycyclin were added per day, the corresponding well served as a control without doxycyclin.

FIG. 7B. Statistical evaluation of the inhibition experiment presented in FIG. 7A

The MFM-223 breast carcinoma cell proliferation data recorded during inhibition by 0.5 mM dTh were subjected a statistical analysis; Box and Whiskers plots are presented

FIG. 8A. Infection of primary prostate carcinoma cells (PC-3) with superTk1 carrying AAV and treatment with 0.5 mM dTh

The bar graph presented in FIG. 8A shows the relative cell proliferation of PC-3 prostate carcinoma cells with and without the infection with superTK1 recombinant Adeno-associated viral vectors without GFP in cis as reporter gene for five days. The bars represent the mean values of cell numbers of triplicates, each sample measured twice, and the error bars display the standard error of the means. All samples were treated with 0.5 mM deoxythymidine (dTh) per day. To one half of the samples rAAV vectors were added at the beginning, the other half served as a control and was not infected.

FIG. 8B. Statistical evaluation of the inhibition experiment presented in FIG. 8A

The PC-3 proliferation data recorded during inhibition by 0.5 mM dTh and pAA driven recombinant superTK1 (recombinant Adeno-associated viral vector) were subjected a statistical analysis; Box and Whiskers plots are presented

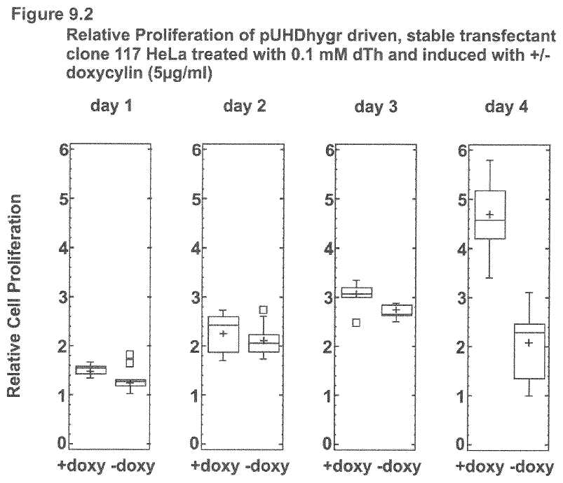

FIG. 9.1: Inhibition of pUHDhygr driven superTk1 stable transfectant Hela clone 117 grown in the presence of 0.1 mM dTh and induced +/−doxycyclin

The histogram shows the growth curve of HeLa117 clone 3 (superTk1 stable transfectant) cultivated in the presence of 0.1 mM dTh (replenished daily) and induced with 5 μg/ml doxycyclin (renewed daily, black bars) or without (white bars). Each sample was measured twice and setup as triplicates.

FIG. 9.2: Box and Whiskers Plot of Inhibition of pUHDhygr driven superTk1 stable transfectant Hela clone 117 grown in the presence of 0.1 mM dTh and induced with/without doxycycline

The Box and Whiskers Plots (FIG. 9.2) show the minimum and maximum (vertical whiskers) as well as the data ranges from 25-75% (grey boxes). Horizontal lines indicate the median and pluses the mean value. Data which are not included in the whiskers are plotted as outliers (small squares).

FIG. 10.1 AB/C/D: Inhibition of pUHDhygr driven superTk1 stable transfectant Hela clone 117 grown in the presence of 0.1 mM dTh induced with/without doxycycline, in the presence of a concentration series (5, 10, 20 and 50 μM, respectively) of 5-FU The histogram shows the growth curve of HeLa117 clone 3 (superTk1 stable transfectant) cultivated in the presence of 0.1 mM dTh and different concentrations (5, 10, 20 and 50 μM, respectively) of 5-fluorouracil (both replenished daily), induced with 5 μg/ml doxycyclin (renewed daily, blue bars) or without (red bars). Each sample was measured twice.

FIG. 10.2: Box and Whiskers Plots of Inhibition of pUHDhygr driven superTk1 stable transfectant clone Hela 117 grown in the presence of 0.1 mM dTh induced with/without doxycycline, in the presence of a concentration series (5, 10, 20 and 50 μM, respectively) of 5-FU

The Box and Whisker Plots show minimum and maximum (top and bottom of the grey boxes) No whiskers are visible due to the low amount of data in the exploratory experiment.

FIG. 11.1 Inhibition of pUHDhygr driven superTk1 stable transfectant clone Hela 117 grown in the presence of 0.1 mM dTh induced with/without doxycycline, in the presence of 5-FU (5 μM)

The histogram shows the growth curve of HeLa117 clone 3 (superTk11 stable transfectant) cultivated in the presence of 0.1 mM dTh and 5 μM 5-fluorouracil (both replenished daily), induced with 5 μg/ml doxycyclin (renewed daily, black bars) or without (white bars). Each sample was set up as triplets and measured twice.

FIG. 11.2: Box and Whiskers Plot of Inhibition of pUHDhygr driven superTk1 stable transfectant clone Hela 117 grown in the presence of 0.1 mM dTh induced with/without doxycycline, in the presence of 5-FU (5 μM)

The Box and Whiskers Plots show the minimum and maximum (vertical whiskers) as well as the data ranges from 25-75% (grey boxes). Horizontal lines indicate the median and pluses the mean value. Data which are not included in the whiskers are plotted as outliers (small squares).

FIG. 11.3: Inhibition of pUHDhygr driven superTk1 stable transfectant Hela clone 117 grown in the presence of 0.1 mM dTh induced with/without doxycycline, in the presence of AraC (5 μM)

The histogram shows the growth curve of HeLa117 clone 3 (superTk1 stable transfectant) cultivated in the presence of 0.1 mM dTh and 5 μM cytarabin (both replenished daily), induced with 5 μg/ml doxycyclin (renewed daily, black bars) or without (white bars). Each sample was set up as triplets and measured twice.

FIG. 11.4: Box-Whisker Plot of Inhibition of pUHDhygr driven superTk1 stable transfectant clone Hela 117 grown in the presence of 0.1 mM dTh induced with/without doxycycline, in the presence of AraC (5 μM)

The Box and Whiskers Plots show the minimum and maximum (vertical whiskers) as well as the data ranges from 25-75% (grey boxes). Horizontal lines indicate the median and pluses the mean value. Data which are not included in the whiskers are plotted as outliers (small squares).

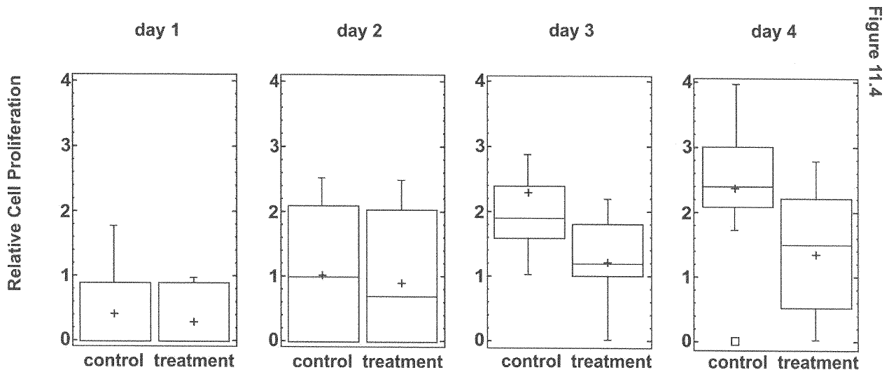

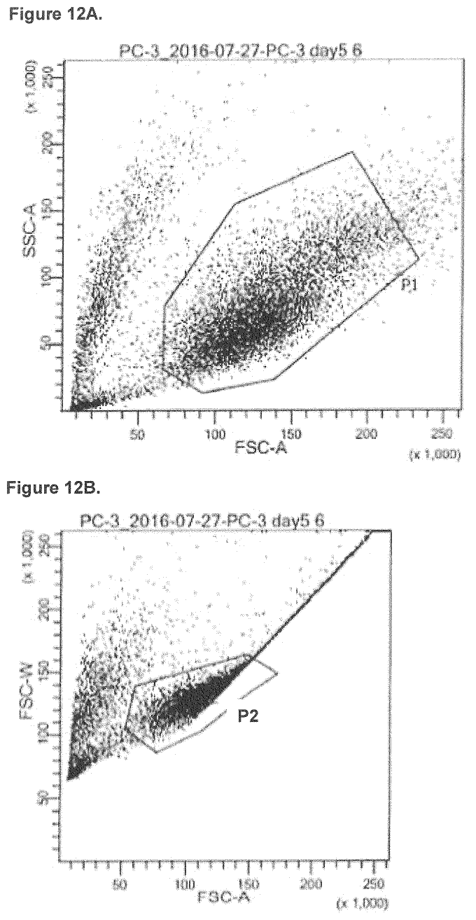

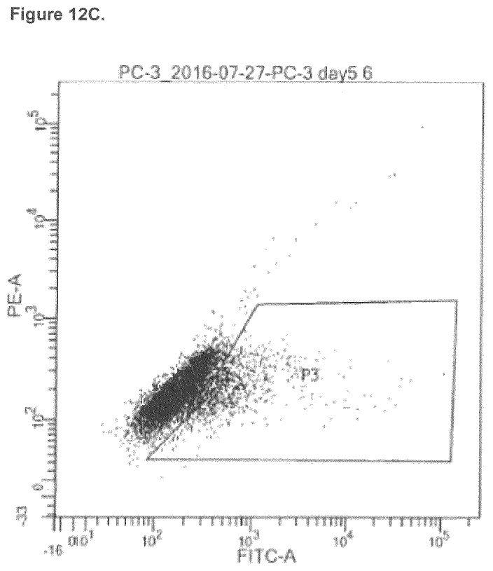

FIG. 12. FACS analysis of PC-3 cells infected with the AAV-Helper Free System with GFP in cis to the superTk1

Legend to FIG. 12ABC: The FACS analysis presented is mainly determined by 2 main values:
Forward scatter (FSC): gives an idea of the size of the cell
Side scatter (SSC): gives an idea of the surface of the cell

FIG. 12A shows the FACS analysis of the P1 gate (living cells); 12B shows the FACS analysis of the P2 gate (living single cells); 12C shows the GFP signal measured in the FITC-A channel; the P3 gate includes all GFP harboring cells. FITC: fluoresceinisothiocyanate; PE=Phycoerythrine

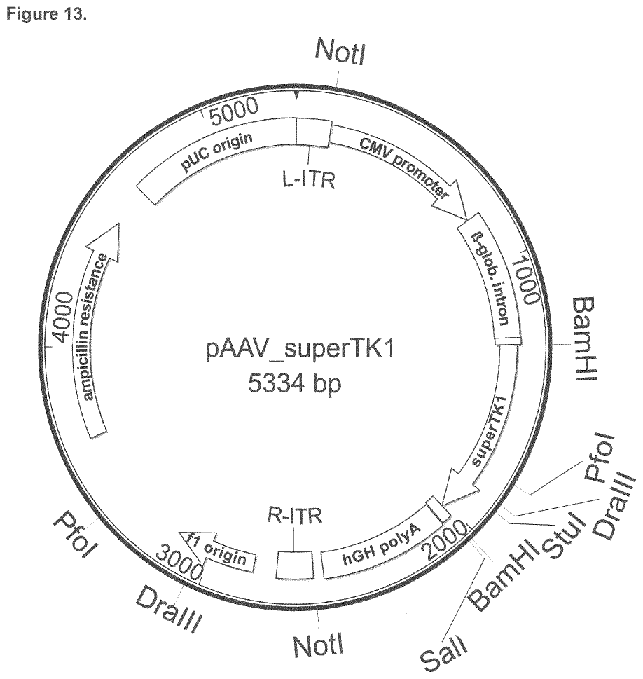

FIG. 13. Plasmid map of pAAVsuperTk1 (no reporter gene GFP attached)

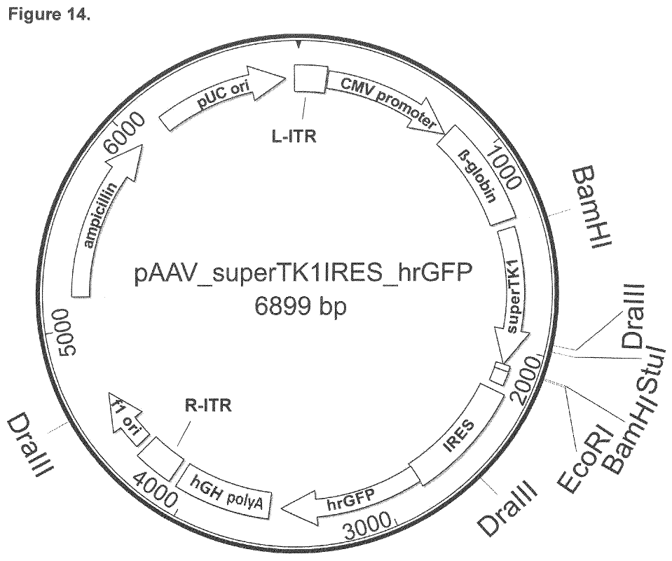

FIG. 14. Plasmid map of pAAVsuperTk1-IRES-hrGFP

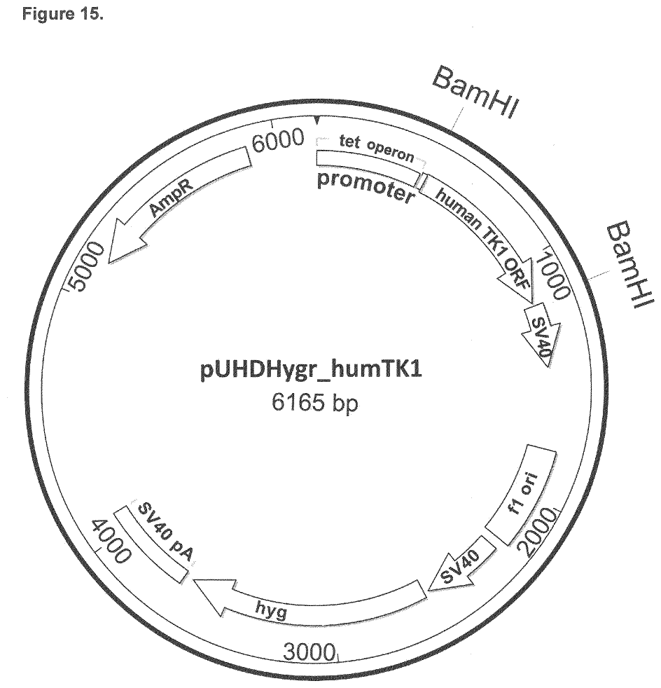

FIG. 15. Plasmid map of pUHDHygr_superTk1

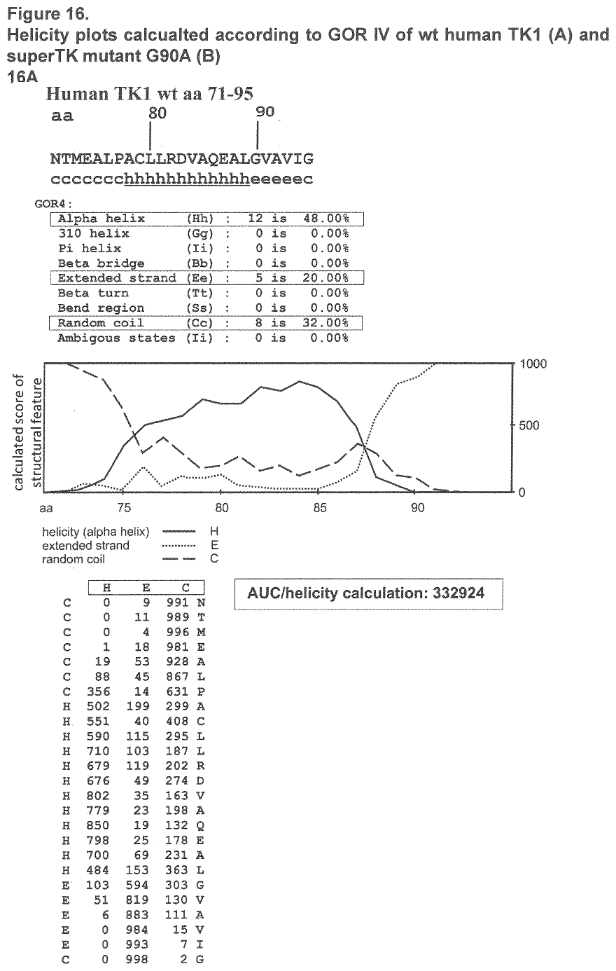

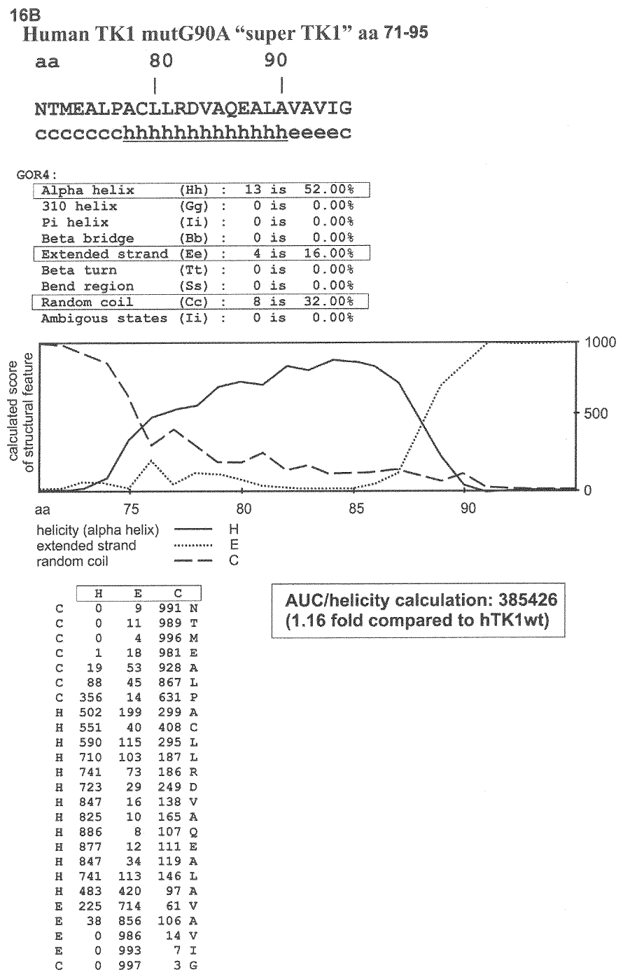

FIG. 16. Helicity plots calculated according to GOR IV of wt human Tk1 (SEQ ID NO: 32) (A) and superTk1 mutant G90A (SEQ ID NO: 38) (B)

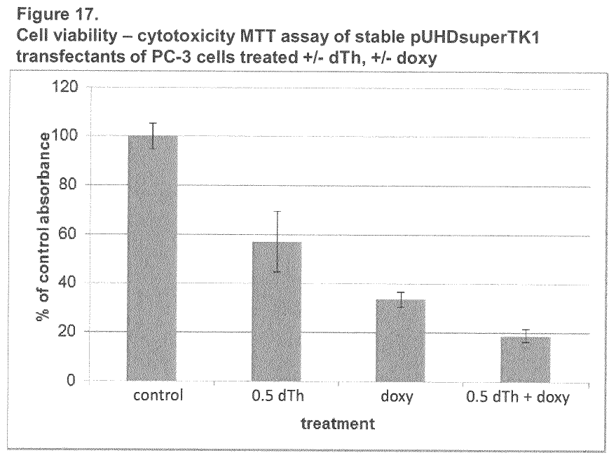

FIG. 17: cell viability—cytotoxicity MTT assay of stable pUHDsuperTK1 transfectants of PC-3 cells treated +/−dTh, +/−doxy

The bar graph shows the percentage of the control absorbance of the converted MTT reagent in PC-3 stable transfectants. The bars show the means of triplicates of the absorbance and the error bars display the standard error of the means.

The mitochondrial activity in untreated PC-3 cells in the control bar on the left defines the control absorbance of the converted dye. Its value was set at 100%. When PC3 cells received 0.5 mM dTh daily for 3 days, their ability to convert the MTT dye was reduced to 55% (second bar from left). The addition of doxycycline instead of dTh to the cell culture medium (third bar) lessened the value to 30%. The combination of both drugs (0.5 dTh+doxy) reduced the mitochondrial activity down to 20% of normal control.

The Examples illustrate the invention.

### EXAMPLE 1: MATERIAL AND METHODS

Cell Lines

Cancer cell lines and cultivation conditions, received from the Leibniz-Institut DSMZ-Deutsche Sammlung von Mikroorganismen und Zellkulturen GmbH, Inhoffenstraße 7B 38124 Braunschweig, Germany

| | |
|---|---|
| Cell line: | PC-3 |
| DSMZ no.: | ACC 465 |
| Species: | Human (homo sapiens) |
| Cell type: | Primary prostate carcinoma |
| Origin: | Established from the bone marrow metastasis isolated |
| | post-mortem from a 62-year-old Caucasian man with |
| | grade IV prostate cancer (poorly differentiated |
| | adenocarcinoma) after androgen suppression therapy; |
| | described to form tumors in nude mice, to grow in |
| | soft agar, and to be unresponsive to androgen |
| | treatment |
| Morphology: | Epithelial-like cells growing adherently in |
| | monolayers or in multilayer foci |
| Medium: | 45% Ham's F12 |
| | 45% RPMI 1640 |
| | 10% FCS h.i |
| | 1× Qmax |
| | 1× P/S |
| Subculture: | Split confluent culture 1:3 to 1:10 every 3-5 days using |
| | trypsin/EDTA; seed out at ca. 1 × 10⁴ cells/cm² |
| Incubation: | At 37° C. |
| Doubling time: | Ca. 50 hours |

| | |
|---|---|
| Cell line: | MFM-223 |
| DSMZ no.: | ACC 57 |
| Species: | Human (homo sapiens) |
| Cell type: | Primary breast carcinoma |
| Origin: | Established from the pleural effusion of a post- |
| | menopausal breast cancer patient without any prior |
| | treatment (tumor: widespread ductal mammary |
| | carcinoma T4 N2 M1 with histological grade III); cells |
| | were described to express androgen, estrogen and |
| | progesterone receptors |
| Morphology: | Adherent cells growing as monolayer with reticulate |
| | growth pattern |
| Medium: | 85% MEM (with Earle's salts) |
| | 15% FCS h.i. |
| | 1x insulin-transferrin-sodium selenite |
| | 1x Qmax |
| | 1x P/S |
| Subculture: | Cells grow very slowly, slit confluent culture 1:2 every |
| | 3-4 days using trypsin/EDTA; seed out at ca. |
| | 1 × 10⁶ cells/25 cm² in 10 ml medium |
| Incubation: | At 37° C. |
| Doubling | Ca. 5-7 days |
| time: | |

| | |
|---|---|
| Cell line: | HeLa |
| DSMZ no.: | ACC 422 |
| Species: | Human (homo sapiens) |
| Cell type: | Cervix carcinoma |
| Origin: | established from the epitheloid cervix carcinoma of a |
| | 31-year-old black woman in 1951; later diagnosis |
| | changed to adenocarcinoma; first aneuploid, |
| | continuously cultured human cell line |
| Morphology: | epithelial-like cells growing in monolayers |
| Medium; | 90% MEM (with Earle's salts + 10% h.i. FBS + 2 mM |
| | L-glutamine + non-essential amino acids (cells also |
| | grow well in 90-95% RPMI 1640 + 5-10% h.i. FBS) |
| | 1x Qmax |
| | 1x P/S |
| Subculture: | split confluent culture 1:4 to 1:6 every 3-5 days using |
| | trypsin/EDTA; cells reach confluence quickly, seed |
| | out at ca. 1-2 × 10⁶ cells/80 cm² |
| Incubation: | At 37° C. |
| Doubling | ca. 48 hours |
| time: | |

SuperTK1

For experiments performed in examples 2-5, human Tk mutG90A sequence (SEQ ID No. 12), here called superTk1, was used. The corresponding nucleotide sequence is shown in FIG. 1 (SEQ ID NO: 30).

Transfection and Isolation of Stable Transfectants of PC-3 and MFM-223 Cells with the Plasmid pUHD superTK1

The Effectene Transfection Reagent (Qiagen) that is a nonliposomal lipid reagent for DNA transfection into a broad range of cell types was used according to the protocol provided by the vendor. For transfection cancer cells were grown to reach 40-80% confluence, then 1.8 μg plasmid DNA were added to buffer EC to reach a final volume of 270 μl. 14.4 μl of the Enhancer were added, the sample was vortexed for 1 second and incubated at room temperature for 5 minutes to allow complex formation. Subsequently the mixture was centrifuged briefly and 45 μl of Effectene Trafo Reagent were added, the solution was mixed on a vortex shaker for 10 seconds and incubated at room temperature for another 5 minutes. Meanwhile, the cells were washed three times with PBS and a new cell culture medium was added. 1.8 ml of cell culture medium were added to the plasmid-DNA mix and mixed by pipetting up and down twice. Then the whole transfection mix was added to the cells. After 48 hours the cells were trypsinized and split 1:3 and to each plate Hygromycin B was added at a final concentration of 100 μg/ml. The transfected cells were cultivated under this selection pressure for at least one month and the growth medium was renewed regularly, always containing HygromycinB, until distinct clones could be detected and isolated.

Isolating Single Clones from Stable Transfectants

The tissue culture plate was carefully washed twice with PBS, a sterilized cloning cylinder was placed around each clone with a tweezer that was sterilized by flaming, and one drop of trypsin was put onto the cells inside the cylinder. Onto the plate some drops of medium were added, so smaller clones stayed alive for harvesting them at a later timepoint. After some minutes detached cells were suspended with growth medium and recovered from the cloning cylinder. Single clones were first propagated in separate wells starting with a 96 well plate and carefully transferred to 24, 12 and finally to 6 well plates as soon as the cells reached confluence.

Isolation of Genomic DNA from Cancer Cells and Transfectants (Modified Version of Following Protocol: [73]

Cells were grown to 50-100% confluence and the cells still attached to the tissue culture plate were washed three times with PBS. Afterwards, 300 μl of digestion buffer (5 mM EDTA, pH 8.0, 200 mM NaCl, 100 mMTris, pH 8.0, 0.2% sodium dodecyl sulfate (SDS)) were added to the cells before harvesting them with a sterile silicone rubber cell scraper. The lysate was transferred into a microfuge tube, 0.4 mg proteinase K per ml digestion buffer were added, and the sample was vortexed and incubated over night at 55° C.

After the proteinase K digestion 1 ml 96% ethanol was added, the sample was vortex and incubated on ice for 1 hour before centrifugation (25000 g, 10 min, 0° C.). Afterwards the ethanol supernatant was discarded, 750 μl of 70% ethanol were added to rinse the pellet which was vortexed and centrifuged again (25 000 g, 10 min., 0° C.). Again, the ethanol supernatant was discarded and the cap of the microfuge tube was left open for the evaporation of remaining alcohol. Subsequently the DNA was resuspended in 100 μl 1×TE buffer (10×: 100 mM Tris, 10 mM EDA) and incubated for approx. 30 min. at 55° C. with the cap opened to allow the ethanol to evaporate. The DNA sample was vortexed repeatedly in between. Then the DNA content was measured using the Nanodrop device at 260 nm.

Genomic PCR (Protocol: Promea Product Information: GoTaqR G2 Colorless Master Mix)

The following primers were used:

superTK1 antisense (pUHDhTk1as):
(SEQ ID NO 46)
5′ ATT AAC CTG CCC ACT GTG CT3′
superTK1 sense (pUHDhumTK1s):
(SEQ ID NO 47)
5′ CTG TGG GGC AAA GAG CTT C 3′
hygromycin sense:
(SEQ ID NO 49)
5′ GAT GTA GGA GGG CGT GGA TA 3′
hygromycin antisense:
(SEQ ID NO 50)
5′ ATA GGT CAG GCT CTC GCT GA 3′

The following primer pair can also be used:

superTK1 antisense (pUHDhTk1as):
(SEQ ID NO 46)
5′ATT AAC CTG CCC ACT GTG CT3′
superTKI sense (pUHDhumTK1s):
(SEQ ID NO 48)
5′CTG TGG GCC AAA GAG CTT C 3′

Procedure: The GoTaqR G2 Colorless Master Mix was thawed at room temperature and kept on ice for further use. The whole reaction was prepared on ice, then homogenized and spun down before it was transferred to the PCR machine.

| | |
|---|---|
| GoTaq^{R} G2 Colorless Master Mix, 2x | 25 μl |
| Primer 1: pUHDhTK1as 10 μM | 5 μl final conc.: 1 μM |
| Primer 2: pUHDhumTK1s 10 μM | 5 μl final conc.: 1 μM |
| Primer 3: hygromycin sense 10 μM | 5 μl final conc.: 1 μM |
| Primer 4: hygromycin antisense 10 μM | 5 μl final conc.: 1 μM |
| DNA template | 3 μg |
| Nuclease free water to | 50 μl |

DNA templates for the controls: Negative control: empty pUHDhygromycin vector: 1 pg positive control: Plasmid pUHDsuperTK1 #117: 1 μg

PCR Protocol:

| | |
|---|---|
| lid | 99° C. |
| beginning | 94° C. for 120 seconds |
| 35 cycles: | 94° C. for 45 seconds |
| | 60° C. for 60 seconds |
| | 72° C. for 60 seconds |
| lid off | 4° C. per second |
| end | 8° C. forever |

Realization of the Growth Experiments:

a) Stable Transfected Clones with pUDHsuperTk1 Treated with Different Amounts of dTh

On day zero 5×10⁴ cells per well of a 6 well plate were plated in 4 ml medium containing hygromycin (0.1 mg/ml): to the upper three wells dTh and doxycycline were added, the lower wells served as controls, only dTh was added. Depending on the number of duplicates and on the incubation time, 5 up to 14 independent 6 well plates were needed. From day one on, the cells were counted at different time points (days) using the Casy Cell Counter (OLS). Triplicates or up to sextuplicates were counted, each measurement was done twice. For a longer time periods independent six well plates had to be set up to generate the necessary cell number of later timepoints, up to eleven days. dTh and doxycycline were added freshly each day at the appropriate concentration and the medium was completely renewed after 72 hours in order to avoid starvation effects. Depending on the growth rate of the cells, the cell number measurements were done daily or every second day. Experiments:
PC-3 clone 2: 0.5 mM dTh—sextuplicates+/−doxycycline (5 μg/ml)—days 1 to 7, measured daily
PC-3 clone 2: 0.1 mM dTh—triplicates+/−doxycycline (5 μg/ml)—days 1 to 4, measured daily
MFM-223 clone 8: 0.5 mM dTh—sextuplicates+/−doxycycline (5 μg/ml) days 1 to 11, measured every second day

b) Human Carcinoma Cells Infected with superTK1 Recombinant Adeno-Associated Viral Vectors

The recombinant Adeno-associated Virus (AAV) expression is a very attractive alternative to the Adenovirus based one because it causes lesser immunological problems, especially after repeated administration in test animals. The helper virus free recombinant adeno-associated virus expression systems were developed to serve as important tools for gene delivery [61]. We used the AAV Helper-Free System distributed by Agilent Technologies [62], which is also the expression system of choice for animal experiments. The components are:
pAAV-MCS vector
pAAV-LacZ vector
pAAV-RC plasmid
pMCS-MCS-vector
pHelper plasmid

The cloning strategy, propagation, and the use of the AAV-system was very straight forward, taking advantage of the BamHI restriction site in the pAAV-MCS vector. By a triple transfection together with the pAAV-RC and pHelper plasmids into HEK-293 cells, high titer AAV virussuperTK1 particles could be produced and isolated by centrifugation. The big advantage of the AAV system is, in addition to causing fewer immunological problems, that all target cells can be infected independent of their state of growth (G0, G1, S, G2 or M-phase). Establishment of the pAAVsuperTK1 carrying constructs in detail:

On day zero 5×10⁴ cells were plated in 2 ml medium per well of a 6 well plate and incubated over night at 37° C. The available viral supernatant was mixed with a 2% heat inactivated FCS containing medium and added to the cells, which were then incubated for 2 hours at 37° C., swirled gently several times in between. To the controls only 1 ml of the medium containing 2% heat inactivated FCS was added (without virus). After the incubation time, 1 ml of medium containing 18% heat inactivated FCS and the desired amount of dTh was added to each well, then the cells were put back to the incubator. Depending on the number of repetitions and on the incubation time, 5 up to 14 independent 6 well plates were set up. From day one on, the cells were counted at different time points (days) using the Casy Cell Counter (OLS). Triplicates or even sextuplicates were counted, each measurement was done twice. The remaining six well plates were incubated for a longer time period to receive the cell number of later timepoints, up to eleven days. dTh was added each day to all wells at the appropriate concentration and the medium was changed after 72 hours. Depending on the growth rate of the cells, the cell number measurements were done daily or every second day. Experiments:
PC-3+virus: 0.5 mM dTh—sextuplicates+/−Virus—days 1 to 7, measured daily (FIG. 8) For the estimation of infected cells a FACS analysis was done on the FACS Fortessa to receive the percentage of GFP emitting, infected cells.

Procedure:

The cells of a well were trypsinized and resuspended in 1 ml medium, 50 μl were taken and diluted in 5 ml of Casy solution and the cells were counted using the Casy device. The remaining cells were put to special tubes for the FACS measurements.

Isolation and Purification of Recombinant AAV Particles:

AAV recombinant particles were purified by a CsCl purification as described with minor modifications [74].

In general recombinant Adeno-associated viral vectors are the system of choice, because they can be easily purified, used in vitro and in vivo, and they do not cause any immunological problems a priori. The CsCl purification protocol has more than one purification step, it contains the lysis of the cells, precipitation of DNA and proteins, ultracentrifugation in a CsCl gradient, followed by dialysis and finally a filtration step is done. In comparison, iodixanol purified AAV vectors, are purer, but CsCl purified vectors contain less empty particles (not even 1%) and are therefore more bioactive.

Growing Adeno Associated Viruses in an Adherent Cell Line: AAV293
AAV293 cells were grown in Dulbecco's modified Eagle's medium, supplemented with 10% FCS, Qmax and P/S (100 U/mL penicillin and 100 μg/mL streptomycin)
Ten 15 cm tissue culture plates were prepared 3 days before transfection: 2×10⁶ cells were seeded per plate
On the day of transfection cells were 70 to 80% confluent
25,384 μg of each plasmid (pHelper, pAAV-RC, recombinant pAAV expression plasmid—dissolved in sterile ddH₂O) were added to 2 ml of 300 mM CaCl2
The DNA-CaCl2 mixture was added dropwise to 2 ml 2×HBS buffer (2× Hepes-buffered saline, 50 mM HEPES, 280 mM NaCl, 1.5 mM Na₂HPO₄)
The mixture was incubated for approx. 2 min at room temperature and then added dropwise to the tissue culture plate while it was swirled gently
After 5 to 6 hours the medium was replaced with a fresh one
The AAV293 cells were then grown for 72 hours at 37° C.

Harvesting of the Adeno Associated Viruses from the Cells:
For harvesting the cells, EDTA was diluted in PBS to get a final concentration of 6.25 mM
The medium of the cells was collected in conical tubes, the culture plates were then washed two times with PBS, afterwards the 6.25 mM EDTA were added to the plates which were then left for approx. 2 min at room temperature. Then, the cells were harvested and put to the conical tubes as well.
The collected cells were centrifuged for 10 min at 1000 g, room temperature.
The supernatant was discarded and the pellets were resuspended in lysis buffer (50 mM Tris, 150 mM NaCl, 2 mM MgCl₂, pH 8.5). For all 10 plates together, 30 ml of lysis buffer were used.
The cells were lysed by three freeze-thaw cycles using a dried ice—ethanol bath and a 37° C. water bath.
For the 10 initially transfected plates, 1000 units of Benzonase were added, the sample was incubated for one hour at 37° C.
Centrifugation 15 min., 2500 g, room temperature: the pellet was discarded and the supernatant was stored at −80° C. for further purification.

CsCl Purification:
300 μl of 2.5M CaCl₂ were added to get a final concentration of 25 mM for pelleting residual DNA
the sample was incubated on ice for one hour
centrifugation: 2500 g, 15 min, 4° C.
The supernatant was transferred to a new tube and the pellet was discarded
40% PEG-8000 was added to get a final concentration of 8% for precipitating proteins including AAVs
incubation on ice for 3 hours
centrifugation 2500 g, 30 min, 4° C.
the supernatant was discarded and the pellet was resuspended in 30 ml resuspension buffer (50 mM HEPES, 150 mM NaCl, 25 mM EDTA, pH 7.4) over night on a tube rotator at 4° C.
if the pellet was not dissolved completely on the next day, a 5 ml Gilson pipette was used for pipetting up and down to resuspend the pellet, then it was left for one more hour on a tube rotator at 4° C..
the sample was centrifuged for 30 min at 2500 g at 4° C.
CsCl was added to the supernatant to reach a 3,149M solution with an RI of 1,3710 (15.8g were added to the viral supernatant)
Ultracentrifuge tubes were filled, weighed and closed and an ultracentrifugation was started: 23 h, 63000 rpm, 21° C. (90Ti rotor)
1 ml fractions were taken from the top of the gradient and the RI was measured
the fractions with an RI between 1,3703 and 1,3758 were collected and dialysed over night against 1×PBS, the buffer was changed altogether 4 times
(the viral supernatants were concentrated using Amicon Ultra 15 centrifugal filter units)
60% glycerol was added to get a final concentration of 10%
the samples were sterile filtered by using Ultra free-CL filter tubes
samples were quick-frozen and stored at −80° C. for further usage

DMEM growth medium: DMEM (4.5 g/L glucose, 110 mg/L sodium pyruvate, 2 mM L-glutamine) 10% (v/v) fetal bovine serum 2 mM L-glutamine

FIG. 12 shows the FACS analysis of PC-3 cells infected with the AAV-Helper Free System with GFP in cis to the superTk1. The P2 gate further helps to identify the population of single cells. In the FITC-A channel the P3 gate surrounds the cells which emit a GFP signal, in the negative control this value has to be zero and the other samples were all compared to the negative control. The reporter GFP was expressed at relatively low levels due to the IRES sequence preceding the hrGFP ORF. Therefore the titer determined may be approximately ten-fold lower than the actual viral titer (manual of the AAV-Helper Free System, Agilent Technologies).

Construction of the Vector pAAV-supTK1-IRES-hrGFP

Digestion of the Empty Vector a AV-IRES-hrGFP with BamH: Reaction Mixture:

| | |
|---|---|
| H₂O | 5 μl |
| NEB 3.1 buffer (conc. 10x) from Biotabs | 3 μl |
| Vector DNA (pAAV-IRES-hrGFP, probe T) | 20 μl for 5 μg |
| Enzyme BamHI (20 U/μl) | 2 μl (=max!) |
| Total amount: | 30 μl |

This reaction was left in the thermo block at 37° C. for one hour.

Treating the Vector AAV-IRES-hrGFP with Alkaline Phosphatase:

Reaction Mixture:

| | |
|---|---|
| Digested vector pAAV-IRES-hrGFP probe T mixture from | All |
| above | 30 μl |
| Antartic reaction buffer 10x | 5 μl |
| ddH₂O | 14 μl |
| Antarctic phosphatase (5 U/μl) | 1 μl |
| Total amount | 50 μl |

The microfuge tube was mixed by slightly “snipping” against its bottom with one finger, then the sample was left for 15 min at 37° C. in the thermo mixer. Afterwards the enzyme was inactivated by incubation for 5 min. at 70° C. before it was frozen at −20° C.

Digesting the Insert supTK1 from the Vector pAAV supTK1 with BamHI:
DNA plasmid pAAV without IRES, containing the wanted insert supTK1, clone number 3:
clone 3 with the correct sequence and correct orientation of the gene was picked: vector length approx. 6 kb, the insert length was about 700 nucleotides [proportion of about one to eight]
for digestion 0.5 to 1 μg insert would have been preferred ⋄ 8 μg of plasmid were needed to get the wanted amount of insert digestion reaction: The reaction mixture was put to the thermo block to 37° C. for one hour.

Checking Vector and Insert on an Agarose Gel:

Vector and insert were loaded onto a 0.9% Agarose gel to check their length and to purify them:

| Slot | sample |
|---|---|
| 1 | 1 kb DNA ladder |
| 2 | Vector pAAV-IRES-hrGFP, sample T, not |
| | digested |
| 3 + 4 | Vector pAAV-IRES-hrGFP BamHI digested |
| | and phosphatase treated |
| 5 | Free |
| 6 | pAAV-supTK1 without digestion |
| 7 | pAAV-supTK1 BamHI digested |
| 8 | pGSX TK1 90GA: old probe which was eluted |
| 9 | pAAV-IRES-OLD: old probe from a vector prep |
| 10 | 100 bp ladder |

Purification of the Vector and Insert from the Gel:

The BamHI cut vector pAAV-IRES-hrGFP and the BamHI cut insert supTK1 were purified from the gel lanes 3, 4 and 7.

Ligation of the Vector and the Insert:

The reaction mixture should be as concentrated as possible, 15-20 μl of volume should be used

| | |
|---|---|
| pAAV-IRES-hrGFP cut with BamHI, phosphatase | 5 μl (0.5 μg) |
| treated | |
| Insert supTK1, cut with BamHi | 12 μl |
| 10x T4 DNA ligase buffer | 2 μl |
| T4 DNA ligase (400 U/μl) | 1 μl |
| Total volume | 20 μl |

Also, a negative control was done: 12 μl ddH₂O instead of the insert were used, the other components stayed exactly the same

The reaction mixtures where put to the 4° C. cold room into a water bath which had 16° C. overnight.

Transformation of the New AAV-supTK1-IRES-hrGFP into Competent E. coli Cells and Checking the Direction of the Insert on an Agarose Gel:

The vector map showing the superTK1 in addition to the hrGFP reporter gene is presented in FIG. 14.

In order to determine the proper insertion of the superTK1 into the pAAV-IRES-hrGFP a restriction digest was performed with EcoRI and StuI. Both enzymes cut only once in the whole new plasmid. EcoRI has a recognition site on the vector backbone close to the superTK1 insert and StuI at the 3′end of it. Dependent on the orientation of the inserted gene, the products of the digestion with both enzymes generated different sizes. The correct orientation of the insert leads to one really large and one really small fragment, the wrong orientation of the insert leads to a “smaller big band” and to a “bigger small band”.

| | |
|---|---|
| Plasmid DNA | 1 μg |
| CutSmart 10x buffer | 2 μl |
| EcoR1-HF (20 U/μl) | 1 μl |
| StuI (10 U/μl) | 1 μl |
| ddH₂O | up to 20 μl |

The digested inserts were analyzed on a 1% agarose gel.

Sequence Analysis:
Sequencing of pAAV_supTK1_IRES_hrGFP, where the gene was inserted in the correct direction: clones 3 and 4 were sent out to Microsynth: twice, 1.2 μg DNA were diluted to get a final volume of 15 μl ⋄ 2 primers—one forward and one reversed—were used for sequence analysis. The sequences were checked using the software MegAlign (DNASTAR-Lasergene): both sequences were correct without any point mutations!

Transfections of AAV-293 Cells with Different Plasmids for Generating Viruses [75]

3 plasmids were used per transfection: The pHelper and the pAAV-RC plasmid were the same all the time, the third plasmid contained the gene of interest in a recombinant vector containing ITR regions and/or GFP as a reporter gene, in addition to superTK1, and, as a control, LacZ alone. These plasmids were used for independent transfections: the recombinant constructs pAAVsuperTK1, pAAV-supTK1-IRES-hrGFP and the plasmid pAAV-LacZ. The pHelper plasmid contains most of the adenovirus genes like E2A, E4 and VA RNA genes, which were needed for producing infective AAV particles (for more details of the recombinant pAAV expression system see [62]).

FIG. 13 shows the plasmid map of pAAVsuperTk1 (no reporter gene GFP attached).

FIG. 14 shows the plasmid map of pAAVsuperTk1-IRES-hrGFP.

FIG. 15 shows the plasmid map of pUHDhygrTk1 expression vector used for the establishment of stable transfectants of either wtTK1 or superTK1.

### EXAMPLE 2: CELL GROWTH AND INHIBITION EXPERIMENTS IN STABLY TRANSFECTED IN HUMAN PRIMARY PROSTATE CARCINOMA (PC-3) AND MAMMARY CARCINOMA (MFM-223) CELLS

In the case of the stable transfectants, superTk1 (Tk1 mutG90A) carried on a plasmid pUDHHyg10.3 was transfected into human prostate PC-3 or human ductal mammary carcinoma cells (MFM-223). Clones of stable transfectants were selected and analyzed for full length superTk1 DNA by specific PCR. Subsequently, growth curves under inhibitory conditions were performed to analyze the cytostatic exertion of superTk1 expression combined with various, very low levels of dTh (0.1 mM) in the growth medium.

First, superTk1 cDNA was integrated into the pUHDhygr expression vector, transfected it into PC-3 cells and selected for stable transfectant clones that present a strong band at 665 nt in a genomic PCR. The proper insert was verified by sequence analysis to make sure that no unwanted point mutation got was introduced during the subcloning procedure (see FIG. 3).

Human Primary Prostate Carcinoma (PC-3) Treated with dTh

FIG. 4 shows stable transfectant clone 2 of primary prostate carcinoma cells (PC-3) with superTk1 carrying pUHDhyg10.3 or wtTk1 and treatment with 0.5 mM dTh (FIGS. 4a and 4c). Relative cell proliferation is expressed as cell number per cells originally seeded. Cells treated with doxycyclin and therefore expressing superTK1 in FIG. 4a do not proliferate and even decrease in cell number from day 4, whereas untreated control cells increase in cell number by a factor 4 until day 7. This indicates that the superTK induced cell cycle arrest effectively prevents tumor cell growth in the presence of the very low concentration of deoxythymidine of 0.5 nM. In comparison, cells carrying wtTk1 induced with doxycyclin in FIG. 4c proliferate nicely, almost as well as the control cells. These results underline that the expression of superTK is necessary for the inhibition of cancer cell proliferation at the given dTh concentration of 0.1 mM.

The Kruskal-Wallis test determines a p-value, which is less than 0.05 from day 2 on; this means that there is a statistically significant difference amongst the medians of the treated (superTk1 expressing sample) sample and its control at the 95.0% confidence level. In this test the method used to discriminate among the means was Fisher's least significant difference (LSD) procedure (cf. FIG. 4B).

| | day1 | day2 | day3 | day4 | day5 | day6 | day7 |
|---|---|---|---|---|---|---|---|
| p value (KrusKal | 0.11847 | 0.01932 | 0.00003 | 0.00003 | 0.00003 | 0.00003 | 0.00003 |
| Wallis, comparison | | | | | | | |
| of the medians) | | | | | | | |

FIG. 4D shows that the separate supply with dTh only (no expression of superTK, only cellular TK is present) or doxycycline only (expression of superTK but no addition of substrate) to the cell culture medium had similar effects on the distribution of the cells in the cell cycle. The portion of cells in the G1 phase was less, whereas the amount of cells in the S and G2/M phase was higher compared to the untreated control cells. For continuous dTh supply it is typical to shift cells into the S phase, but obviously the addition of doxycycline had similar effects in this cell line.

The administration of both drugs simultaneously, which induced the expression of superTK and supplied the substrate, had the strongest effect onto the cell cycle distribution of the cells. No cells were detectable in the G2/M phase, approx. one fourth of the cells was present in the G1 phase. Over 75% of the cells were measured while undergoing the S phase. Due to the addition of dTh and doxycycline in combination, the cells stopped dividing, they rested in the S phase and did not proliferate anymore.

FIG. 5 shows stable transfectant clone 13 of primary prostate carcinoma cells (PC-3) with superTk1 carrying pUHDHyg10.3 and treatment with 0.1 mM dTh.

The growth curves of PC-3 cells treated with 0.1 mM dTh showed a very similar growth inhibition as seen with 0.5 mM dTh presented in FIG. 4. With a daily supply of 0.1 mM dTh plus doxycyclin induction, a p-value below 0.05 could be deduced from the proliferation data from day 3 on. This statistical fact clearly underlines the observation, that superTK expression inhibits tumor cell proliferation. The combination therapy of doxycycline with 0.1 mM dTh did not allow the cells to proliferate at all within the four observed days. One Way Anova results acquired with Stat Graphics: the Kruskal-Wallis test shows a p-value, which is less than 0.05 from day 3 on; this means that there is a statistically significant difference amongst the medians of the treated sample and its control at the 95.0% confidence level. The Multiple Range Test identifies 2. different groups and shows a statistically significant difference at the 95.0% confidence level as well from day 3 on. The method used to discriminate among the means is Fisher's least significant difference (LSD) procedure (cf. FIG. 5B).

| | day1 | day2 | day3 | day4 |
|---|---|---|---|---|
| p value (Kruskall Wallis, | 0.12753 | 0.07764 | 0.00648 | 0.00395 |
| comparison of the | | | | |
| medians) | | | | |

Human Mammary Carcinoma (MFM223) Cells Treated with dTh

The MFM-223 pUHDsuperTK1 clone 8 was chosen for the growth experiments because it was the fastest growing clone of this slowly growing cell line despite having only a weaker superTK1 band compared to others (such as clone 16 in lane 7) at the correct length of approx. 665 bp on a 1.2% Agarose gel (see black box); see FIG. 6. FIG. 6 shows an agarose gel analysis of genomic PCR-results of stable transfected clones with pUDHsuperTk1 in MFM-223 cells.

FIG. 7 shows stable transfectant clone 8 of primary MFM-223 Breast Carcinoma with superTk1 carrying pUHDHyg10.3 and treatment with 0.5 mM dTh.

Comparable to the PC-3 prostate carcinoma cells in FIG. 4. the MFM-233 mammary carcinoma cells stopped to proliferate when treated with 0.5 mM dTh in combination with doxycyclin induction. Even the cell number continuously decreased during the treatment period of 11 days. A very similar result could be observed when 0.1 mM dTh were used (data not shown). The Kruskal-Wallis test determines a p-value, which is less than 0.05 from day 1 on; this means that there is a statistically significant difference amongst the medians of the treated sample and its control at the 95.0% confidence level; cf. FIG. 7B.

| | day1 | day3 | day5 | day7 | day9 | day11 |
|---|---|---|---|---|---|---|
| p value | 0.00556 | 0.00009 | 0.00003 | 0.00003 | 0.00003 | 0.00003 |
| (Kruskall Wallis, | | | | | | |
| comparison of | | | | | | |
| the medians) | | | | | | |

FIG. 8 shows infection of primary prostate carcinoma cells (PC-3) with superTk1 carrying AAV and treatment with 0.5 mM dTh.

After the infection with the rAAVs the cell proliferation was slowed down during the treatment period of 5 days. Similar to the transfected and genomically integrated superTK1 in FIGS. 4 and 5, in the case of the viral AAv expression system, where the target cells had to be infected with recombinant superTK1 AAV viruses, the total number of PC-3 cells continuously decreased during the observation period. Hence, one can conclude that tumor cells infected with the viral expression construct rAAV-superTK1 could not proliferate during the five days of treatment with 0.5 mM dTh.

The Kruskal-Wallis test determines a p-value, which is less than 0.05 from day 1 on; this means that there is a statistically significant difference amongst the medians of the treated sample and its control at the 95.0% confidence level; cf. FIG. 8B.

| | day1 | day2 | day3 | day4 | day5 |
|---|---|---|---|---|---|
| p value (Kruskall | 0.01631 | 0.00648 | 0.00395 | 0.00388 | 0.00388 |
| Wallis, comparison | | | | | |
| of the medians) | | | | | |

### EXAMPLE 3: HUMAN HELA CELLS (CERVIX CARCINOMA) TREATED WITH DTH AND WITH ARAC OR 5-FU IN COMBINATION

One of the major aims was to find out the lowest dTh concentration still able to cause a growth halt in tumor cells stable transfected with superTK1. For this task this gene was integrated in pUHDhygr and stable transfected into HeLa cervix carcinoma cells. One of the clones showing the highest amount of superTk1 cDNA was clone 117[76] These transfected HeLa cells were grown under permanent hygromycin and puromycin selection pressure for 4 to 7 days, starting with 50.000 cells/well (12.500 cells/ml). In order to establish a perfect growth curve cell counts on a daily basis with a Casy cell counting system were mandatory. Tumor cell cultures expressing the super TK1 and cultivated under 0.1 mM dTh and doxycyclin induction still showed a clear growth repression.

That means even 1/50 of standard dTh concentration was sufficient for the superTk1 to cause a S-phase block in HeLa cells (FIG. 9.1).

The Kruskal-Wallis test determines a p-value which is less than 0.05 except for days 1 and 2; this means that there is a statistically significant difference amongst the medians of the treated samples versus its controls at the 95.0% confidence level.; cf. FIG. 9.2.

In addition to growth inhibition with dTh alone we wanted to explore the synergistic effects on tumor cell growth in HeLa clone 117 superTk1 transfectants when one of the nucleoside analogues, cytarabine and 5-fluorouracil in very low concentrations was applied. At first, concentration series of 5, 10, 20 and 50 μM as well as 1, 2, 5 and 5 μM AraC and 5-FU, respectively, were set up in order to investigate the lowest concentration still able to cause cell growth arrest when combined with dTh treatment. In both cases, the extremely low concentrations of 5 μM, seemed to be the most sufficient ones for AraC or 5′-FU (see FIGS. 10.1A and 11).

In this case now it is of interest to extrapolate the cell culture conditions with serum levels currently given during a clinical treatment. For the case of 5-FU, a regular therapeutic cytostatic dosis in cancer treatment is 400 mg/m2/day or a serum level, which corresponds to 55.44 μg/ml cₘₐₓ in the serum (according to: [77]). Our working concentration 5 μM results in a calculated serum concentration of 0.65 μg/ml.

This means that there still is a statistically significant inhibition with a 1/85 of the amount of 5-FU used in the literature. For AraC the values are comparable. (see FIG. 11)).

The biggest impact appeared to be evident on day 4, where the cell number of the doxycyclin induced superTk1 affected cells was reduced almost to half of the amount of the control without doxycyclin induction in both the AraC and 5-FU triplets. In this experiment, cytarabin appeared to be the drug with the higher cell growth arrest potential. In a direct comparison of the control groups without doxycycline treatment, the relative cell proliferation on day 4 reached only 2.4 times the number of day 1 in the cytarabin group. Whereas in the case of the 5-FU-group, a 3.3-fold higher cell number was reached on day 4 compared to day 1. Another concentration series was set up, with 5, 2.5 and 1 μM AraC or 5-FU, respectively. The concentrations lower than 5 μm of the chemotherapeutics obviously were too low to exert any inhibitory effects.

One Way Anova results were acquired with Stat Graphics: Since the experiment was thought of as an exploratory one, each sample was set up only once (one 6-well plate) and measured twice-thus it was not possible to determine meaningful p-values. The Multiple Range Test identifies 2 different groups and shows a statistically significant difference level from day 3-4 (5 μM 5-FU), day 2 (10 μM 5-FU), none (20 μM 5-FU) and day 4 (50 μM 5-FU). In this test the method used to determinate among the means was Fisher's least significant difference (LSD) procedure; see FIG. 10.2.

Since the experiment was planned as an exploratory one, each sample was set up only once (one 6-well plate) and measured twice—thus it was not possible to determine meaningful p-values. The Multiple Range Test identifies 2 different groups and shows a statistically significant difference level on day 4 (5 μM AraC), day 1-2 (10 μM AraC), day 2 (20 μM AraC) and none (50 μM AraC). In this test the method used to determinate among the means was Fisher's least significant difference (LSD) procedure; (data not shown)

FIG. 11 shows the inhibition of pUHDhygr driven superTk1 stable transfectant clone Hela 117 grown in the presence of 0.1 mM dTh induced with/without doxycycline, in the presence of 5-FU (5 μM).

The Kruskal-Wallis test determines a p-value which is less than 0.05 on days 3 and 4; this means that there is a statistically significant difference amongst the medians of the treated samples versus its controls at the 95.0% confidence level; cf. FIG. 11.2 and FIG. 11.4.

### EXAMPLE 4: CALCULATION OF HELICITY DETERMINATION OF ACTIVITY OF WILD-TYPE AND MUTANT HUMAN THYMIDIN KINASES

The helicity calculations of the protein domain aa 71-95 of the wt human TK1 and the mutations were done using GOR IV. [78] Results are presented in FIG. 16A for the wt human TK1 and in FIG. 16B for the mutant G90A. In applying the GORIV software application, scores for each type secondary structure feature were calculated. In the case of this helical domain those are alpha helical, extended strand, and random coil. In order to generate a possibility to compare the helicity in this sequence element, the surface area underneath the helical plot was integrated.

As a result of this procedure the mutant G90A is characterized by a 1.16 fold increase of helicity in relation to the wtTK1 (FIG. 16B). Exactly the same procedure was imposed on all the other human TK1 mutants, giving the increases of helicity presented in table 1.

Other methods for secondary structure prediction can be used, e.g.: SOPM [79], SOPMA [80], HNN [81], MLRC [81], DPM [82], DSC [83], GOR I [84], GOR III [85], PHD [86], PREDATOR [87], or SIMPA96 [88].

All GOR IV helicity calculations were performed by Network Protein Sequence Analysis, provided from the PBIL-IBCP Institute of Biology and Protein Chemistry 7, passage of Vercors 69367 Lyon Cedex 07, FRANCE TIBS [41] https://npsa-prabi.ibcp.fr/cgibin/npsa_automat.pl?page=/NPSA/npsa_seccons.html

FIG. 16 The helicity scores of the protein domain aa 71-95 of the wt human TK1 and the superTK1 were done using GOR IV. [68] The area under the curve was determined by IrfanView (Microsoft Windows). Area under the curve for wt TK1 was calculated to be 332924, for super TK1 a value of 385426 was calculated and divided by the value of wt Tk1, resulting in a relative helicity of 1.16. The increase in surface area is a direct measure for the increase of helicity in the aa domain of aa 71 to aa 95.

Calculation of TK Activity

For the determination of a specific activity a quantification of the protein solution was done first, according the method of Bradford. [43] The dye stock solution (Biorad) containing methanol and acetic acid was diluted 1:4 with ddH2O. An aliquot of the protein solution was then filled up to 1 ml with the diluted Bradford solution. (Usually 5l of protein solution are diluted in 995 ml Bradford solution. The protein concentration is calculated by multiplying the absorbance at =595 nm with 17 and dividing this by 5, resulting in the concentration in μg/μl.) Depending on the concentration of the kinases 5-20 μl of the sample solution were used.

The following components were mixed for the enzyme assay:

10 fold mix:
0.5 M Tris-HCl, pH 7.5
0.1 M DTT
25 mM ATP
25 mM MgCl₂

Set up for the activity test: 5.0 μl 10-fold mix
2.5 μl BSA (60 mg/ml)
1.0 μl Chaps (25 mM)
1.0 μl NaF (0.15 M)
1.0 μl ³H-dTh or 3H-dCyd (500 μM, 2 Ci/mmol)
5.0-20 μl of the protein solution
Filled up with ddH₂O to 50 μl

The set-up was incubated at 37° C. At distinct times 10 μl aliquots were removed from the reaction mix and pipetted onto small pieces of DE81-filters. After removal of every aliquot, the filter papers were washed in a big volume of ammonium formiat (5 mM for TK assay), transferred to water and rinsed, and finally dried after shortly immersing them in ethanol. Then, after the dried filters were transferred into the scintillation tubes, the bound radioactive nucleotides were eluted by addition of 500 μl elution buffer (0.1 M HCl, 0.2 M KCl). 2.5 ml of scintillation solution were added before measuring the radioactivity. A standard was created in order to convert the cpm-values into pmol: 5 μl from some of the reactions were removed and pipetted on DE81-filters, which were not washed, but directly transferred to the scintillation tubes, treated like described above, and measured. 5 μl of the standard contained 50 pMol of the substrate or the product. From the cpm-values of these samples one got the converting factor UF (is equal to the specific activity of the used substrate): UF=cpm standard/50 pMol

For the calculation of the whole enzymatic activity of one fraction (U) the following formula was used:

$$U = \frac{Δ cpm . 50 μ l . Vol}{10 μ l . 20 μ l . UF}$$

10 μl were pipetted at each time point onto the filter, and 20 μl of the protein solution was initially used. cpm=(cpmt2−cpm t1)/t2−t1 (cpm/min); t1=start of the reaction; t2=end of the reaction, e.g. 5 min; Vol=total volume of the fraction (μl); UF is the specific activity (see above) (cpm/pMol); U is the whole activity of the enzyme per fraction (pMol/min×μl=Mol/min×ml).

TABLE 2
Correlation of the calculated protein secondary structure parameter
(degree of helicity) with the measured specific enzyme activity of
human cytosolic Tk1 mutants with elevated and one with decreased
specific activity

| humTK1- | Helicity | measured specific Tk1 | | Relative in-/decrease |
|---|---|---|---|---|
| variant | (AUC) | enzyme activity | | of helicity to wt Tk1 |
| wt | 332924 | 37 | pmol/mg × min | 1.00 |
| L8081F | 255025 | 1.4 | pmol/mg × min | 0.77 |
| G90A | 385426 | 375 | pmol/mg × min* | 1.16 |
| V84DG90A | 366809 | 393 | pmol/mg × min | 1.10 |

*data retrieved from a different experiment

Legend to table 2: Helicity scores given in the table were calculated by NPS@: Network Protein Sequence Analysis [42]; the AUC was obtained by integration of the area under the helicity curve between aa 71-95 of the TK1 variants and equals the degree of helicity of the protein domain

### EXAMPLE 5: CELL VIABILITY—CYTOTOXICITY MTT ASSAY OF STABLE PUHDSUPERTK1 TRANSFECTANTS OF PC-3 CELLS TREATED +/−DTH, +/−DOXY

The MTT assay is a colorimetric assay for assessing cell metabolic activity; see J Immunol Methods. 1983 Dec. 16; 65(1-2):55-63; Sigma Aldrich. (2016). MTT Cell Viability Applications. The control shows the mitochondrial activity in untreated PC-3 cells with endogenous wild-type TK1 only, without addition of dTh or induction of superTK1 (FIG. 17, left bar, (“control”)). Its value was set at 100%. When PC-3 cells received 0.5 mM dTh daily for 3 days, their ability to convert the MTT dye was reduced to 55% (FIG. 17, second bar from left). This indicates a partial inhibition of their mitochondrial activity due to the activity of the endogenous wild-type TK1. The addition of doxycycline to induce the expression of superTK1 to the cell culture medium (third bar) showed the profound effect of superTK1 on cell viability even without addition of dTh, i.e. using endogenous dTh levels as substrate (cell viability decreased to approx. 30% compared to control); see FIG. 17, 2nd bar from the right (“doxy”)).—Addition of dTh even further increased the effect of superTK1 on cell viability (cell viability decreased to approx. 20% compared to control); see FIG. 17, right bar (“0.5 dTh+doxy”). By contrast, endogenous wild-type TK1 in addition to the non-induced but baseline expression of the pUDHencoded superTK1 had only a moderate effect on cell viability even when dTh was added; see FIG. 17, 2nd bar from the left (“0.5 dTh”).

### EXAMPLE 6: VALIDATION IN ANIMAL MODELS

For this experiment human Tk1 mutG90A sequence (SEQ ID No. 12), here called superTk1, is used. The corresponding nucleotide sequence is shown in FIG. 1 (SEQ ID NO: 30).

Experimental Approach 1

SuperTK1 carrying glioblastoma cells (3×10⁶/50 μl; stable transfected) are subcutaneously implanted as xenografts in SCID mice. Due to the fact that all cells carry the superTK1 integrated into the genomic DNA, every single tumor cell is targeted by the induction of the recombinant superTK1 by doxycyclin and thus harmed by gene therapy. After 2 weeks of tumor growth in the breast region of the animal, the therapy is started by adding doxycyclin to the drinking water. In parallel, osmotic mini pumps are implanted in the neck of the SCID mice, prefilled with the necessary amount of dTh and, for some test animals with the cytostatics AraC or 5′-FU as well, if the test animal is to be treated by a combination therapy.

After 3 weeks of therapy regimen, the successful treatment is analyzed by physical, histological, and molecular analyses. In the same manner, untransfected and previously untreated glioblastoma cells are implanted and function as controls.

Number of test animals for this milestone:
1) 3 mice for the glioblastoma cell line without any treatment (control)
2) pUDHsuperTK1 transfected glioblastoma cells treated with 0.1 mM dTh alone (3 mice)
3) pUDHsuperTK1 transfected glioblastoma cells treated with 0.1 mM dTh plus 0.5 μmM AraC (3 mice)
4) pUDHsuperTK1 transfected glioblastoma cells treated with 0.1 mM dTh plus 0.5 μmM 5′-FU (3 mice)
5) pUDH-0 (empty vector control) transfected glioblastoma cells treated with 0.1 mM dTh alone (3 mice)

In total 15 test animals are needed for this experimental approach

Experimental Approach 2:

Based on the results of the first experiment, in this experiment previously untreated glioblastoma cells are implanted in SCID mice (3×10⁶/100 μl). After development of about 2 weeks, the solid tumor is analyzed by MRT to get a most precise 3D-information about the size and structure of the tumor. Usually, the solid tumor has grown to a size of 6-10 mm. The infection with superTK1-AAV recombinant particles is exactly planned and based on the stereoscopic data. The infection is accomplished by infiltration of the solid tumor at several recombinant virus infusion areas. The necessary infiltration spots as well as the total amounts in MOI/μl are optimized by pretests with ˜8-10 SCID mice. For this purpose, rAAVsuperTK1-GFP constructs are used that already have been generated and characterized by FACS analyses. They allow the visual tracing of successful infection by green fluorescence protein expression. The aim is to generate highly concentrated recombinant AAVsuperTK1 particles with a high MOI in order to be able to reduce the necessary liquid amounts to a minimum and avoid generating bulbs. On the other hand, it is essential to infect not only the tumor cells but also the stroma. The artificial expression is induced up to 3 weeks/therapy cycle. The tumor cell survival rate is evaluated by physical, cell- and molecular biological analyses. The histo-immuno-cytological examinations are performed in a similar way as described for the first approach in the histolab of MFPL.

Necessary test animals for the second milestone:
1) 10 mice for the pretests to optimize the rAAVsuperTK1 infection/infiltration process 2) the 3 untreated control mice have already been analyzed in approach 1
3) pAAVsuperK1 infected glioblastoma cells treated with 0.1 mM dTh treatment (3 mice)
4) pAAVsuperK1 infected glioblastoma cells treated with 0.1 mM dTh alone (3 mice)
5) pAAVsuperK1 infected glioblastoma cells treated with 0.1 mM dTh plus 0.5 μmM AraC (3 mice)
6) pAAVsuperK1 infected glioblastoma cells treated with 0.1 mMdTh plus 0.5 μmM 5′-FU (3 mice) In total 22 test animals are needed for this experimental approach.

### REFERENCES

1. Stritzker, J., et al., Inducible Gene Expression in Tumors Colonized by Modified Oncolytic Vaccinia Virus Strains. Journal of Virology, 2014. 88(19): p. 11556-11567.
2. Glinka, E. M., Eukaryotic expression vectors bearing genes encoding cytotoxic proteins for cancer gene therapy. Plasmid, 2012.68(2): p. 69-85.
3. Agu, C. A., et al., The cytotoxic activity of the bacteriophage lambda-holin protein reduces tumour growth rates in mammary cancer cell xenograft models. J Gene Med, 2006.8(2): p. 229-41.
4. Kircheis, R., et al., Tumor-targeted gene delivery: an attractive strategy to use highly active effector molecules in cancer treatment. Gene Ther, 2002.9(11): p. 731-5.
5. Gamsjäger, M., Molecular analysis of a novel cytotoxic gene: Transmembrane protein 66 (Tmem66). 2012, Medical University of Vienna.
6. Munch-Petersen, B., et al., Human thymidine kinase 1. Regulation in normal and malignant cells. Adv Enzyme Regul, 1995.35: p. 69-89.
7. Boone, C., T. R. Chen, and F. H. Ruddle, Assignment of three human genes to chromosomes (LDH-A to 11, TK to 17, and IDH to 20) and evidence for translocation between human and mouse chromosomes in somatic cell hybrids (thymidine kinase-lactate dehydrogenase A-isocitrate dehydrogenase-C-11, E-17, and F-20 chromosomes). Proc Natl Acad Sci USA, 1972.69(2): p. 5104.
8. Fain, P. R., E. Solomon, and D. H. Ledbetter, Second international workshop on human chromosome 17. Cytogenet Cell Genet, 1991.57(2-3): p. 66-77.
9. Willecke, K., et al., Human mitochondrial thymidine kinase is coded for by a gene on chromosome 16 of the nucleus. Somatic Cell Genet, 1977.3(3): p. 237-45.
10. Munch-Petersen, B. and G. Tyrsted, Induction of thymidine kinases in phytohaemagglutinin-stimulated human lymphocytes. Biochim Biophys Acta, 1977. 478(3): p. 364-75.
11. He, Q., S. Skog, and B. Tribukait, Cell cycle related studies on thymidine kinase and its isoenzymes in Ehrlich ascites tumours. Cell Prolif, 1991.24(1): p. 3-14.
12. Gasparri, F., et al., Thymidine kinase 1 expression defines an activated G1 state of the cell cycle as revealed with site-specific antibodies and ArrayScan assays. Eur J Cell Biol, 2009.88(12): p. 779-85.
13. Sherley, J. L. and T. J. Kelly, Regulation of human thymidine kinase during the cell cycle. J Biol Chem, 1988.263(17): p. 8350-8.
14. Kristensen, T., H. K. Jensen, and B. Munch-Petersen, Overexpression of human thymidine kinase mRNA without corresponding enzymatic activity in patients with chronic lymphatic leukemia. Leuk Res, 1994. 18(11): p. 861-6.
15. Luo, P., et al., Thymidine kinase activity in serum of renal cell carcinoma patients is a useful prognostic marker. Eur J Cancer Prev, 2009. 18(3): p. 220-4.
16. Zhang, J., et al., Thymidine kinase 1: a proliferation marker for determining prognosis and monitoring the surgical outcome of primary bladder carcinoma patients. Oncol Rep, 2006.15(2): p. 455-61.
17. Jagarlamudi, K. K., L. O. Hansson, and S. Eriksson, Breast and prostate cancer patients differ significantly in their serum Thymidine kinase 1 (TK1) specific activities compared with those hematological malignancies and blood donors: implications of using serum TK1 as a biomarker. BMC Cancer. 15: p. 66.
18. Aufderklamm S1, T. T., Gakis G, Kruck S, Hennenlotter J, Stenzl A, Schwentner C., Thymidine kinase and cancer monitoring. Cancer Lett. 2012 March; 316(1):6-10, 2012. 316(1): p. 6-10.
19. Hallek M, W. L., Strohmeyer S, Emmerich B., Thymidine kinase: a tumor marker with prognostic value for non-Hodgkin's lymphoma and a broad range of potential clinical applications. Ann Hematol., 1992.65(1): p. 1-5.
20. Nisman B, A. T., Kaduri L, Maly B, Gronowitz S, Hamburger T, Peretz T., Serum thymidine kinase 1 activity in breast cancer. Cancer Biomark., 2010.7(2): p. 65-72.
21. Fujiwaki R, H. K., Nakayama K, Moriyama M, Iwanari O, Katabuchi H, Okamura H, Sakai E, Miyazaki K., Thymidine kinase in epithelial ovarian cancer: relationship with the other pyrimidine pathway enzymes. Int J Cancer., 2002. 20(99(3)): p. 328-335.
22. Martin D S, S. R., Sawyer R C, Nayak R, Spiegelman S, Young C W, Woodcock T., An overview of thymidine. Cancer, 1980. 15(45 (5 Suppl)): p. 1117-1128.
23. Kirkwood, J. M., et al., Comparison of pharmacokinetics of 5-fluorouracil and 5-fluorouracil with concurrent thymidine infusions in a Phase 1 trial. Cancer Res, 1980. 40(1): p. 107-13.
24. Harris, A. W., E. C. Reynolds, and L. R. Finch, Effect of thymidine on the sensitivity of cultured mouse tumor cells to 1-beta-D-arabinofuranosylcytosine. Cancer Res, 1979. 39(2 Pt 1): p. 538-41.
25. Osswald, H., [Overadditive synergism of combinations of cyclophosphamide with thymidine, adenosine or uridine in transplantation tumors]. Arzneimittelforschung, 1972.22(7): p. 1184-8.
26. O'Dwyer, P. J., et al., Role of thymidine in biochemical modulation: a review. Cancer Res, 1987.47(15): p. 3911-9.
27. Fox, R. M., E. H. Tripp, and M. H. N. Tattersall, Mechanism of Deoxycytidine Rescue of Thymidine Toxicity in Human T-Leukemic Lymphocytes. Cancer Research, 1980.40(5): p. 1718-1721.
28. Rankin P W, J. E., Benjamin R C, Moss J, Jacobson M K, Quantitative studies of inhibitors of ADP-ribosylation in vitro and in vivo. J Biol Chem. 1989 Mar. 15; 264(8):4312-7., 1989. 15(264): p. 43312-4317.
29. Trucco C, O. F., de Murcia G, Mdnissier-de Murcia J, DNArepair defect in poly (ADP-ribose)polymerase-deficient cell lives. Nucleic Acid Res, 1998. 126: p. 2644-2649.
30. Kufe D W, B. P., Karp D, Parker L, Rosowsky A, Canellos G, Frei E 3rd, High-dose thymidine infusions in patients with leukemia and lymphoma. Blood. April; 55(4):580-9, 1980 55(4): p. 580-589.
31. Blumenreich M S, W. T., Andreeff M, Hiddemann W, Chou T C, Vale K, O'Hehir M, Clarkson B D, Young C W., Effect of very high-dose thymidine infusions on leukemia and lymphoma patients. Cancer Res., 1984.44(5): p. 2203-2207.
32. Chiuten D F, W. P., Zaharko D S, Edwards L, Clinical phase I-II and pharmacokinetic study of high-dose thymidine given by continuous intravenous infusion. Cancer Res., 1980.40(3): p. 818-822.
33. Robins H I, T. K., Katschinski D M, Jacobson E, Mehta M, Olsen M, Cohen J D, Tiggelaar C L, Arzoomanian R Z, Alberti D, Feierabend C, Wilding G., Phase I trial of intravenous thymidine and carboplatin in patients with advanced cancer. J Clin Oncol. September, 1999. 17(9): p. 2922-2931.
34. Andrei, G. and R. Snoeck, Emerging drugs for varicella-zoster virus infections. Expert Opin Emerg Drugs, 2011. 16(3): p. 507-35.
35. Johnson, V. A. and M. S. Hirsch, New developments in antiretroviral drug therapy for human immunodeficiency virus infections. AIDS Clin Rev, 1990: p. 235-72.
36. Schmidt, S., et al., Sequence Analysis of Herpes Simplex Virus 1 Thymidine Kinase and DNA Polymerase Genes from over 300 Clinical Isolates from 1973 to 2014 Finds Novel Mutations That May Be Relevant for Development of Antiviral Resistance. Antimicrob Agents Chemother, 2015.59(8): p. 4938-45.
37. Budik, S., Recombinant expression of a super versus feeble human thymidine kinase 1 (TK) and amino/carboxyterminal subfragments. 2000, University of Vienna.
38. Kufe, D. W., et al., Thymidine arrest and synchrony of cellular growth in vivo. Cancer Treat Rep, 1980.64(12): p. 1307-17.
39. Denizot, F. and R. Lang, Rapid colorimetric assay for cell growth and survival. Modifications to the tetrazolium dye procedure giving improved sensitivity and reliability. J Immunol Methods, 1986. 89(2): p. 271-7.
40. Budik, S., Recombinant expression of a super versus feeble human thymidine kinase1 (TK) and amino/carboxy-terminal subfragments. 2000, University of Vienna.
41. Combet, C., Blanchet, C., Geourjon, C., Deleage, G., NPS@: Network Protein Sequence Analysis. Trends in Biochemical Sciences, 2000.25(3): p. 147-150.
42. Piper, A. A., M. H. N. Tattersall, and R. M. Fox, The Activities of Thymidine Metabolizing Enzymes during the Cell-Cycle of a Human-Lymphocyte Cell-Line Laz-007 Synchronized by Centrifugal Elutriation. Biochimica Et Biophysica Acta, 1980.633(3): p. 400-409.
43. Bradford, M. M., A rapid and sensitive method for the quantitation of microgram quantities of protein utilizing the principle of protein-dye binding. Anal Biochem, 1976. 72: p. 248-54.
44. Combet, C., et al., NPS@: network protein sequence analysis. Trends Biochem Sci, 2000.25(3): p. 147-50.
45. Lee, S. S., B. C. Giovanella, and J. S. Stehlin, Jr., Selective lethal effect of thymidine on human and mouse tumor cells. J Cell Physiol, 1977.92(3): p. 401-5.
46. Kufe, D. W., et al., High-dose thymidine infusions inpatients with leukemia and lymphoma. Blood, 1980.55(4): p. 580-9.
47. Fried, J., et al., Cytotoxic and cytokinetic effects of thymidine, 5-fluorouracil, and deoxycytidine on HeLa cells in culture. Cancer Res, 1981.41(7): p. 2627-32.
48. Cohen, A. and B. Ullman, Analysis of the drug synergism between thymidine and arabinosyl cytosine using mouse S49 T lymphoma mutants. Cancer Chemother Pharmacol, 1985. 14(1): p. 70-3.
49. Yin, H., et al., Non-viral vectors for gene-based therapy. Nat Rev Genet, 2014.15(8): p. 541-55.
50. Hoyng, S. A., et al., Developing a potentially immunologically inert tetracycline-regulatable viral vector for gene therapy in the peripheral nerve. Gene Ther, 2014. 21(6): p. 549-57.
51. Gossen, M., et al., Transcriptional activation by tetracyclines in mammalian cells. Science, 1995.268(5218): p. 1766-9.
52. Gossen, M. and H. Bujard, Tight control of gene expression in mammalian cells by tetracycline-responsive promoters. Proc Natl Acad Sci USA, 1992.89(12): p. 5547-51.
53. Olins, D. E., A. L. Olins, and P. H. Von Hippel, Model nucleoprotein complexes: studies on the interaction of cationic homopolypeptides with DNA. J Mol Biol, 1967. 24(2): p. 157-76.
54. Laemmli, U. K., Characterization of DNA condensates induced by poly(ethylene oxide) andpolylysine. Proc Nat Acad Sci USA, 1975. 72(11): p. 4288-92.
55. Jefferson, A., V. E. Cadet, and A. Hielscher, The mechanisms of genetically modified vaccinia viruses for the treatment of cancer. Crit Rev Oncol Hematol, 2015.95(3): p. 407-16.
56. Du, P., et al., Lentivirus-Mediated RNAi silencing targeting ERCCI reverses cisplatin resistance in cisplatin-resistant ovarian carcinoma cell line. DNA Cell Biol, 2015. 34(7): p. 497-502.
57. Song, Z., et al., RNAi-mediated downregulation of CDKL1 inhibits growth and colony-formation ability, promotes apoptosis of human melanoma cells. J Dermatol Sci, 2015. 79(1): p. 57-63.
58. Hagen, J., J. P. Scheerlinck, and R. B. Gasser, Knocking down schistosomes—promise for lentiviral transduction in parasites. Trends Parasitol, 2015.31(7): p. 324-32.
59. Yee, J. K. and J. A. Zaia, Prospects for gene therapy using HIV-based vectors. Somat Cell Mol Genet, 2001.26(1-6): p. 159-74.
60. Chen, H., et al., A novel adenoviral vector carrying an all-in-one Tet-On system with an autoregulatory loop for tight, inducible transgene expression. BMC Biotechnol, 2015. 15: p. 4.
61. Grimm, D. and J. A. Kleinschmidt, Progress in adeno-associated virus type 2 vector production: promises and prospects for clinical use. Hum Gene Ther, 1999. 10(15): p. 2445-50.
62. AAV Helper-Free System (Instruction Manual). 2017, Agilent Technologies.
63. Cole-Strauss, A., et al., Correction of the mutation responsible for sickle cell anemia by an RNA-DNA oligonucleotide. Science, 1996.273(5280): p. 1386-9.
64. Sambrook, J. R., D. W., Molecular Cloning: A Laboratory Manual. 2001: CSHL Press.
65. Ausubel, F. M., Current Protocols in Molecular Biology. 1988: Green Publishing Associates and Wiley Interscience, N. Y.
66. Hames, B. D. H., S. J., Nucleic acid hybridisation—A practical approach. 1985: IRL Press, Oxford.
67. Thompson, J. D., D. G. Higgins, and T. J. Gibson, CLUSTAL W: improving the sensitivity of progressive multiple sequence alignment through sequence weighting, position-specific gap penalties and weight matrix choice. Nucleic Acids Res, 1994.22(22): p. 4673-80.
68. Brutlag, D. L., et al., Improved sensitivity of biological sequence database searches. Comput Appl Biosci, 1990.6(3): p. 237-45.
69. Altschul, S. F., et al., Gapped BLAST and PSI-BLAST: a new generation of protein database search programs. Nucleic Acids Res, 1997.25(17): p. 3389-402.
70. Altschul, S. F., A protein alignment scoring system sensitive at all evolutionary distances. J Mol Evol, 1993.36(3): p. 290-300.
71. Altschul, S. F., et al., Basic local alignment search tool. J Mol Biol, 1990.215(3): p. 403-10.
72. Henikoff, S. and J. G. Henikoff, Amino acid substitution matrices from protein blocks. Proc Nat Acad Sci USA, 1992. 89(22): p. 10915-9.
73. Wang, Z. and D. R. Storm, Extraction of DNA from mouse tails. Biotechniques, 2006. 41(4): p. 410,412.
74. Strobel, B., et al., Comparative Analysis of Cesium Chloride-and Iodixanol-Based Purification of Recombinant Adeno-Associated Viral Vectors for Preclinical Applications. Hum Gene Ther Methods, 2015.26(4): p. 147-57.
75. Stritzker, J., et al., Inducible gene expression in tumors colonized by modified oncolytic vaccinia virus strains. J Virol, 2014.88(19): p. 11556-67.
76. Sack, S., Design and put to the test of superactive human thymidine kinases 1 integrated in a eukaryotic plasmid bound expression system pUHDHygr. 2017, Medical University of Vienna.
77. Casale, F., et al., Plasma concentrations of S-fluorouracil and its metabolites in colon cancer patients. Pharmacol Res, 2004.50(2): p. 173-9.
78. Gamier, J. G., J.-F.; Robson, B., GOR secondary structure prediction method version IV, in Methods in Enzymology, R. F. Doolittle, Editor. 1996. p. 540-553.
79. Geourjon, C. and G. Deleage, Sopm—a Self-Optimized Method for Protein Secondary Structure Prediction. Protein Engineering, 1994.7(2): p. 157-164.
80. Geourjon, C. and G. Deleage, SOPMA: Significant improvements in protein secondary structure prediction by consensus prediction from multiple alignments. Computer Applications in the Biosciences, 1995. 11(6): p. 681-684.
81. Guermeur, Y., et al., Improved performance in protein secondary structure prediction by inhomogeneous score combination. Bioinformatics, 1999. 15(5): p. 413-421.
82. Deleage, G. and B. Roux, An Algorithm for Protein Secondary Structure Prediction Based on Class Prediction. Protein Engineering, 1987. 1(4): p. 289-294.
83. King, R. D. and M. J. E. Sternberg, Identification and application of the concepts important for accurate and reliable protein secondary structure prediction. Protein Science, 1996.5(11): p. 2298-2310.
84. Gamier, J., D. J. Osguthorpe, and B. Robson, Analysis of Accuracy and Implications of Simple Methods for Predicting Secondary Structure of Globular Proteins. Journal of Molecular Biology, 1978. 120(1): p. 97-120.
85. Gibrat, J. F., J. Gamier, and B. Robson, Further Developments of Protein Secondary Structure Prediction Using Information-Theory—New Parameters and Consideration of Residue Pairs. Journal of Molecular Biology, 1987. 198(3): p. 425-443.
86. Rost, B. and C. Sander, Prediction of Protein Secondary Structure at Better Than 70-Percent Accuracy. Journal of Molecular Biology, 1993.232(2): p. 584-599.
87. Frishman, D. and P. Argos, Incorporation of non-local interactions in protein secondary structure prediction from the amino acid sequence. Protein Engineering, 1996.9(2): p. 133-142.
88. Levin, J. M., Exploring the limits of nearest neighbour secondary structure prediction. Protein Eng, 1997. 10(7): p. 771-6.

All references cited herein are fully incorporated by reference. Having now fully described the invention, it will be understood by a person skilled in the art that the invention may be practiced within a wide and equivalent range of conditions, parameters and the like, without affecting the spirit or scope of the invention or any embodiment thereof.

The present invention refers to the following nucleotide and amino acid sequences: The wild-type sequences provided herein are available in the NCBI database and can be retrieved from www.ncbi.nlm.nih.gov/sites/entrez?db=gene; Theses sequences also relate to annotated and modified sequences. The present invention also provides techniques and methods wherein homologous sequences, and variants of the concise sequences provided herein are used.

Preferably, such “variants” are genetic variants.

```
SEQ ID No. 1:
Human TK1 mutM73AA75DD83AG9095A nt sequence
                                                                  atgagctgcattaacctgcccactgtgctg
241 cccggctcccccagcaagacccgggggcagatccaggtgattctcgggccgatgttctca
301 ggaaaaagcacagagttgatgagacgcgtccgtcgcttccagattgctcagtacaagtgc
361 ctggtgatcaagtatgccaaagacactcgctacagcagcagcttctgcacacatgaccgg
421 aacaccgccgaggacctgcccgcctgcctgctccgagccgtggcccaggaggccctggct
481 gtggctgtcatagctatcgacgaggggcagtttttccctgacatcgtggagttctgcgag
541 gccatggccaacgccgggaagaccgtaattgtggctgcactggatgggaccttccagagg
601 aagccatttggggccatcctgaacctggtgccgctggccgagagcgtggtaaagctgacg
661 gcggtgtgcatggagtgcttccgggaagccgcctataccaagaggctcggcacagagaag
721 gaggtcgaggtgattgggggagcagacaagtaccactccgtgtgtcggctctgctacttc
781 aagaaggcctcaggacagcctgccgggccggacaacaaagagaactgcccagtgccagga
841 aagccaggggaagccgtggctgccaggaagctctttgccccacagcagattctgcaatgg
901 atccagacatga
SEQ ID No. 2:
Human TK1 mutM73AA75DD83AG9095A aa sequence
    1 MSCINLPIVL PGSPSKTRGQ IQVILGPMFS GKSTELMRRV RRFQIAQYKC
  51 LVIKYAKDTR YSSSFCTKDR NTAEDLPACL LRAVAQEALA VAVIAIDEGQ
101 FFPDIVEFCE AMANAGKIVI VAALDGIFQR KPFGAILNLV PLAESVVKLT
151 AVCMECFREA AYTKRLGTEK EVEVIGGADK YKSVCRLCYF KKASGUAGP
201 DNKENCPVPG RPGEAVAARK LEAPQQILQW IQT
SEQ ID No. 3:
Human TK1 mutM73AA75DD83AG90A nt sequence
                                                                  atgagctgcattaacctgaccactgtgctg
241 cccggctcccccagcaagacccgggggcagatccaggtgattctcgggccgatgttctca
301 ggaaaaagcacagagttgatgagacgcgtccgtcgcttccagattgctcagtacaagtgc
361 ctggtgatcaagtatgccaaagacactcgctacagcagcagcttctgcacacatgaccgg
421 aacaccgccgaggacctgcccgcctgcctgctccgagccgtggcccaggaggccctggct
481 gtggctgtcataggcatcgacgaggggcagtttttccctgacatcgtggagttctgcgag
541 gccatggccaacgccgggaagaccgtaattgtggctgcactggatgggaccttccagagg
601 aagccatttggggccatcctgaacctggtgccgctggccgagagcgtggtgaagctgacg
661 gcggtgtgcatggagtgcttccgggaagccgcctataccaagaggctccgcacagagaag
721 gaggtcgaggtgattgggggagcagacaagtaccactccgtgtgtcggctctgctacttc
781 aagaaggcctcaggccagcctgccgggccggacaacaaagagaactgcccagtgccagga
841 aagccaggggaagccgtggctgccaggaagctctttgccccacagcagattctgcaatgg
901 atccagacatga
SEQ ID No. 4:
Human TK1 mutM73AA75DD83AG90A aa sequence
    1 MSCINLPTVL PGSPSKTRGQ IQVILGPMFS GKSTELMRRV RRFQIAQYKC
  51 LVIKYAKDTR YSSSECTHDR NTAEDLPACL LRAVAQEALA VAVIGIDEGQ
101 FFPDIVEFCE AMANAGKTVI VAALDGTFQR KPFGAILNLV PLAESVVKLI
151 AVCMECFREA AYTKRLGTEK EVEVIGGADK YHSVCRLCYF KKASGQPAGP
201 DNKENCPVPG RPGEAVAARK LFAPGQILQW IQT
SEQ ID No. 5:
Human TK1 mutV84EG90A nt sequence
                                                                  atgagctgcattaacctgcccactgtgctg
241 cccggctcccccagcaagacccgggggcagatccaggtgattctcgggccgatgttctca
301 ggaaaaagcacagagttgatgagacgcgtccgtcgcttccagattgctcagtacaagtgc
361 ctggtgatcaagtatgccaaagacactcgctacagcagcagcttctgcacacatgaccgg
421 aacaccatggaggcgctgcccgcctgcctgctccgagacgaggcccaggaggccctggct
481 gtggctgtcataggcatcgacgaggggcagtttttccctgacatcgtggagttctgcgag
541 gccatggccaacgccgggaagaccgtaattgtggctgcactggatgggaccttccagagg
601 aagccatttggggccatcctgaacctggtgccgctggccgagagcgtggtgaagctgacg
661 gcggtgtgcatggagtgcttccgggaagccgcctataccaagaggctcggcacagagaag
721 gaggtcgaggtgattgggggagcagacaagtaccactccgtgtgtcggctctgctacttc
781 aagaaggcctcaggccagcctgccgggccggacaacaaagagaactgcccagtgccagga
841 aagccaggggaagccgtggctgccaggaagctctttgccccacagcagattctgcaatgg
901 atccagacatga
SEQ ID No. 6:
Human TK1 mutV84EG90A aa sequence
    1 MSCINLPTVL PGSPSKTRGQ IQVILCPMFS GKSTELMRRV RRFQIAQYKC
  51 LVIKYAKDTR YSSSFCTEDR NTMEALPACL LRDEAQEALA VAVIGIDEGQ
101 FFPDIVEFCE AMANAGKTVI VAALDGTFQR KPFGAILNLV PLAESVVKLT
151 AVCMECFREA AYTKKLGTEK EVEVIGGADK YHSVCRLCYF KKASGQPAGP
201 DNKENCPVPG RPGEAVAARK LFAPQQILQW IQT
SED ID No. 7:
Human TK1 mutMT73AA75DG9095A nt sequence
                                                                  atgagctgcattaacctgcccactgtgctg
241 cccggctcccccagcaagacccgggggcagatccaggtgattctcgggccgatgttctca
301 ggaaaaagcacagagttgatgagacgcgtccgtcgcttccagattgctcagtacaagtgc
361 ctggtgatcaagtatgccaaagacactcgctacagcagcagcttctgcacacatgaccgg
421 aacaccgccgaggacctgcccgcctgcctgctccgagacgtggcccaggaggccctggct
481 gtggctgtcatagctatcgacgaggggcagtttttccctgacatcgtggagttctgagag
541 gccatggccaacgccgggaagaccgtaattgtggatgcactggatgggaccttccagagg
601 aagccatttggggccatcctgaacctggtgccgctggccgagagcgtggtgaagctgacg
661 gcggtgtgcatggagtgcttccgggaagccgcctataccaagaggctcggcacagagaag
721 gaggtcgaggtgattgggggagcagacaagtaccactccgtgtgtcggctctgctacttc
781 aagaaggcctcaggccagcctgccgggccggacaacaaagagaactgcccagtgccagga
841 aagccaggggaagccgtggctgccaggaagctctttgccccacagcagattctgcaatgg
901 atccagacatga
SEQ ID No. 8:
Human TK1 mutM73AA75DG9095A aa sequence
    1 MSCINLPTVL PGSPSKTRGQ IQVILGPMFS GKSTELMRRV RRFQIAQYKC
  51 LVIKYAKDTR YSSSFCTHDR NTAEDLPACL LRDVAQEALA VAVIAIDEGQ
101 FFPDIVEFCE AMANAGKTVI VAALDGTFQR KPFGAILNLV PLAESVVKLT
151 AVCMECFREA AYTKRLGTEK EVEVIGGADK YHSVCRLCYF KKASGQPAGP
201 DNKENCPVPG RPGEAVAARK LFAPQQILQW IQT
SEQ ID No. 9:
Human TK1 mutM73AA75DG90A nt sequence
                                                                  atgagatgcattaacctgcccactgtgctg
241 cccggctcccccagcaagacccgggggcagatccaggtgattctcgggccgatgttctca
301 ggaaaaagcacagagttgatgagacgcgtccgtcgcttccagattgctcagtacaagtgc
361 ctggtgatcaagtatgccaaagacactcgctacagcagcagcttctgcacacatgaccgg
421 aacaccgccgaggacctgcccgcctgcctgctccgagacgtggcccaggaggccctggct
481 gtggctgtcataggcatcgacgaggggcagtttttccctgacatcgtggagttctgcgag
541 gccatggccaacgccgggaagaccgtaattgtggctgcactggatgggaccttccagagg
601 aagccatttggggccatcctgaacctggtgccgctggccgagagcgtggtgaagctgacg
661 gcggtgtgcatggagtgcttccgggaagccgcctataccaagaggctcggcacagagaag
721 gaggtcgaggtgattgggggagcagacaagtaccactccgtgtgtcggctctgctacttc
781 aagaaggcctcaggccagcctgccgggccggacaacaaagagaactgcccagtgccagga
841 aagccaggggaagccgtggctgccaggaagctctttgccccacagcagattctgcaatgg
901 atccagacatga
SEQ ID No. 10:
Human TK1 mutM73AA75DG90A aa sequence
    1 MSCINLPTVL PGSPSKTRGQ IQVILGPMES GKSTELMRRV RRFQIAQYKC
  51 LVIKYAKDTR YSSSFCTHDR NTAEDLPACL LRDVAQEALA VAVIGIDEGQ
101 FFPDIVEFCE AMANAGKTVI VAALDGTFQR KPFGAILNLV PLAESVVKLT
151 AVCMECFREA AYTKRLGTEK EVEVIGGADK YHSVCRLCYF KKASGQPAGP
201 DNKENCPVPG RPGEAVAARK LFAPQQILQW IQT
SEQ ID No. 11:
Human TK1 mutG90A nt sequence
                                                                  atgagctgcattaacctgcccactgtgctg
241 cccggctcccccagcaagacccgggggcagatccaggtgattctcgggccgatgttctca
301 ggaaaaagcacagagttgatgagacgcgtccgtcgcttccagattgctcagtacaagtgc
361 ctggtgatcaagtatgccaaagacactcgctacagcagcagcttctgcacacatgaccgg
421 aacaccatggaggcgctgcccgcctgcctgctccgagacgtggcccaggaggccctggct
481 gtggctgtcataggcatcgacgaggggcagtttttccctgacatcgtggagttctgcgag
541 gccatggccaacgccgggaagaccgtaattgtggctgcactggatgggaccttccagagg
601 aagccatttggggccatcctgaacctggtgccgctggccgagagcgtggtgaagctgacg
661 gcggtgtgcatggagtgcttccgggaagccgcctataccaagaggctcggcacagagaag
721 gaggtcgaggtgattgggggagcagacaagtaccactccgtgtgtcggctctgctacttc
781 aagaaggcctcaggccagcctgccgggccggacaacaaagagaactgcccagtgccagga
841 aagccaggggaagccgtggctgccaggaagctctttgccccacagcagattctgcaatgg
901 atccagaacatga
SEQ ID No. 12: “human superTK1”
Human TK1 mutG90A aa sequence
    1 MSCINLPTVL PGSPSKTRGQ IQVILGPMFS GKSTELMRRV RRFQIAQYKC
  51 LVIKYAKDTR YSSSFCTHDR NTMEALPACL LRDVAQEALA VAVIGIDEGQ
101 FFPDIVEFCE AMANAGKTVI VAALEGTFQR KPFGAILNLV PLAESVVKLT
151 AVCMECFREA AYTKRLGTEK EVEVIGGADK YHSVCRLCYF KKASGQPAGP
201 DNKENCPVPG RPGEAVAARK LFAPQQILQW IQT
SEQ ID No. 13:
Human TK1 mutA75DG90E nt sequence
                                                                  atgagctgcattaacctgcccactgtgctg
211 cccggctcccccagcaagacccgggggcagatccaggtgattctcgggccgatgttctca
301 ggaaaaagcacagagttgatgagacgcgtccgtcgcttccagattgctcagtacaagtgc
361 ctggtgatcaagtatgccaaagacactcgctacagcagcagcttctgcacacatgaccgg
421 aacaccatggaggacctgcccgcctgcctgctccgagacgtggcccaggaggccctggag
481 gtggctgtcataggcatcgacgaggggcagtttttccctgacatcgtggagtictgcgag
541 gccatggccaacgccgggaagaccgtaattgtggctgcactggatgggaccttccagagg
601 aagccatttggggccatcctgaacctggtgccgctggccgagagcgtggtgaagctgacg
661 gcggtgtgcatggagtgcttccgggaagccgcctataccaagaggctcggcacagagaag
721 gaggtcgaggtgattgggggagcagacaagtaccactccgtgtgtcggctctgctacttc
781 aagaaggcctcaggccagcctgccgggccggacaacaaagagaactgcccagtgccagga
841 aagccaggggaagccgtggctgccaggaagctctttgccccacagcagattctgcaatgg
901 atccagacatga
SEQ ID No. 14:
Human TK1 mutA75DG90E aa sequence
    1 MSCINLPTVL PGSPSKTRGQ IQVILGPMFS GKSTELMRRV RRFQIAQYKC
  51 LVIKYAKDTR YSSSFCTHDR NTMEDLPACL LRDVAQEALE VAVIGIDEGQ
101 FFPDIVEFCE AMANAGKTVI VAALDGTFQR KPFGAILNLV PLAESVVKLT
151 AVCMECFREA AYTKRLGTEK EVEVIGGADK YHSVCRLCYF KKASGQPAGP
201 DNKENCPVPG RPGEAVAARK LFAPQQILQW IQT
SEQ ID No, 15:
Human TK1 mutV84DG90A nt sequence
                                                                  atgagctgcattaacctgcccactgtgctg
241 cccggctcccccagcaagacccgggggcagatccaggtgattctcgggccgatgttctca
301 ggaaaaagcacagagttgatgagacgcgtccgtcgcttccagattgatcagtacaagtgc
361 ctggtgatcaagtatgccaaagacactcgctacagcagcagcttctgcacacatgaccgg
421 aacaccatggaggcgctgcccgcctgcctgctccgagacgacgcccaggaggccctggct
481 gtggctgtcataggcatcgacgaggggcagtttttccctgacatcgtggagttctgcgag
541 gccatggccaacgccgggaagaccgtaattgtggctgcactggatgggaccttccagagg
601 aagccatttggggccatcctgaacctggtgccgctggccgagagcgtggtgaagctgacg
661 gcggtgtgcatggagtgcttccgggaagccgcctataccaagaggctcggcacagagaag
721 gaggtcgaggtgattgggggagcagacaagtaccactccgtgtgtcggctctgctacttc
781 aagaaggcctcaggccagcctgccgggccggacaacaaagagaactgcccagtgccagga
841 aagccaggggaagccgtggctgccaggaagctctttgccccacagcagattctgcaatgg
901 atccagacatga
SEQ ID Na 16:
Human TK1 mutV84DG90A aa sequence
    1 MSCINLPTVL PGSPSKTRGQ IQVILGPMFS GKSTELMRRV RRFQIAQYKC
  51 LVIKYAKDTR YSSSFCTHDR NTMEALPACL LRDDAQEALA VAVIGIDEGQ
101 FFPDIVEFCE AMANAGKTVI VAALDGTFQR KPFGAILNLV PLAESVVKLT
151 AVCMECFREA AYTKRLGTEK EVEVIGGADK YHSVCRLCYF KKASGQPAGP
201 DNKENCPVPG RPGEAVAARK LFAPQQILQW IQT
SEQ ID No. 17:
Human TK1 mutA75GA84EG90A nt sequence
                                                                  atgagctgcattaacctgcccactgtgctg
241 cccggctcccccagcaagacccgggggcagatccaggtgattctcgggccgatgttctca
301 ggaaaaagcacagagttgatgagacgcgtccgtcgcttccagattgctcagtacaagtgc
361 ctggtgatcaagtatgccaaagacactcgctacagcagcagcttctgcacacatgaccgg
421 aacaccatggaaggcctgcccgcctgcctgctccgagacgaggcccaggaggccctggct
481 gtggctgtcataggcatcgacgaggggcagtttttccctgacatcgtggagttctgcgag
541 gccatggccaacgccgggaagaccgtaattgtggctgcactggatgggaccttccagagg
601 aagccatttggggccatcctgaacctggtgccgctggccgagagcgtggtgaagctgacg
661 gcggtgtgcatggagtgcttccgggaagccgcctataccaagaggctcggcacagagaag
721 gaggtcgaggtgattgggggagcagacaagtaccactccgtgtgtcggctctgctacttc
781 aagaaggcctcaggccagcctgccgggccggacaacaaagagaactgcccagtgccagga
841 aagccaggggaagccgtggctgccaggaagctctttgccccacagcagattctgcaatgg
901 atccagacatga
SEQ ID No. 18:
Human TK1 mutA75GV84EG90A aa sequence
    1 MSCINLPTVL PGSPSKTRGQ IQVILGPMFS GKSTELMRRV RRFQIAQYKC
  51 LVIKYAKDTR YSSSFCTHDR NTMEGLPACL LRDEAQEALA VAVIGIDEGQ
101 FFPDIVEFCE AMANAGKTVI VAALDGTFQR KPFGAILNLV PLAESVVKLT
151 AVCMECFREA AYTKRLGTEK EVEVIGGADK YHSVCRLCYF KKASGQPAGP
201 DNKENCPVPG RPGEAVAARK LFAPQQILQW IQT
SEQ ID No. 19:
Human TK1 mutG90D nt sequence
                                                                  atgagctgcattaacctgcccactgtgatg
241 cccggctcccccagcaagacccgggggcagatccaggtgattctcgggccgatgttctca
301 ggaaaaagcacagagttgatgagacgcgtccgtcgcttccagattgctcagtacaagtgc
361 ctggtgatcaagtatgccaaagacactcgctacagcagcagcttctgcacacatgaccgg
421 aacaccatggaggcgctgcccgcctgcctgctccgagacgtggcccaggaggccctggac
481 gtggctgtcataggcatcgacgaggggcagtttttccctgacatcgtggagttctgcgag
541 gccatggccaacgccgggaagaccgtaattgtggctgcactggatgggaccttccagagg
601 aagccatttggggccatcctgaacctggtgccgctggccgagagcgtggtgaagctgacg
661 gcggtgtgcatggagtgcttccgggaagccgcctataccaagaggctcggcacagagaag
721 gaggtcgaggtgattgggggagcagacaagtaccactccgtgtgtcggctctgctacttc
781 aagaaggcctcaggccagcctgccgggccggacaacaaagagaactgcccagtgccagga
841 aagccaggggaagccgtggctgccaggaagctctttgccccacagcagattctgcaatgg
901 atccagacatga
SEQ ID No. 20:
Human TK1 mutG90D aa sequence
    1 MSCINLPTVL PGSPSKTRGQ IQVILGPMFS GKSTELMRRV RRFQIAQYKC
  51 LVIKYAKDTR YSSSFCTHDR NTMEALPACL LRDVAQEALD VAVIGIDEGQ
101 FFPDIVEFCE AMANAGKTVI VAALDGTFQR KPFGAILNLV PLAESVVKLT
151 AVCMECFREA AYTKRLGTEK EVEVIGGADK YHSVCRLCYF KKASGQPAGP
201 DNKENCPVPG RPGEAVAARK LFAPQQILQW IQT
SEQ ID No. 21:
Human TK1 mutG90V nt sequence
                                                                  atgagctgcattaacctgcccactgtgctg
241 cccggctcccccagcaagacccgggggcagatccaggtgattctcgggccgatgttctca
301 ggaaaaagcacagagttgatgagacgcgtccgtcgcttccagattgctcagtacaagtgc
361 ctggtgatcaagtatgccaaagacactcgctacagcagcagcttctgcacacatgaccgg
421 aacaccatggaggcgctgcccgcctgcctgctccgagacgtggcccaggaggccctggtg
481 gtggctgtcataggcatcgacgaggggcagtttttccctgacatcgtggagttctgcgag
541 gccatggccaacgccgggaagaccgtaattgtggctgcactggatgggaccttccagagg
601 aagccatttggggccatcctgaacctggtgccgctggccgagagcgtggtgaagctgacg
661 gcggtgtgcatggagtgattccgggaagccgcctataccaagaggctcggcacagagaag
721 gaggtcgaggtgattgggggagcagacaagtaccactccgtgtgtcggctctgctacttc
781 aagaaggcctcaggccagcctgccgggccggacaacaaagagaactgcccagtgccagga
841 aagccaggggaagccgtggctgccaggaagctctttgccccacagcagattctgcaatgg
901 atccagacatga
SEQ ID No. 22:
Human TK1 mutG90V aa sequence
    1 MSCINLPTVL PGSPSKTRGQ IQVILGPMFS GKSTELMRRV RREQIAQYKC
  51 LVIKYAKDTR YSSSFCTHDR NTMEALPACL LRDVAQEALV VAVIGIDEGQ
101 FFPDIVEFCE AMANAGKTVI VAALDGTFQR KPFGAILNLV PLAESVVKLT
151 AVCMECFREA AYTKRLGTEK EVEVIGGADK YHSVCRLCYF KKASGQPAGP
201 DNKENCPVPG RPGEAVAARK LFAPQQILQW IQT
SEQ ID No. 23:
Human TK1 mutA75QV84E nt sequence
                                                                  atgagctgcattaacctgcccactgtgctg
241 cccggctcccccagcaagacccgggggcagatccaggtgattctcgggccgatgttctca
301 ggaaaaagcacagagttgatgagacgcgtccgtcgcttccagattgctcagtacaagtgc
361 ctggtgatcaagtatgccaaagacactcgctacagcagcagcttctgcacacatgaccgg
421 aacaccatggagcagctgcccgcctgcctgctccgagacgaggcccaggaggccctgggc
481 gtggctgtcataggcatcgacgaggggcagtttttccctgacatcgtggagttctgcgag
541 gccatggccaacgccgggaagaccgtaattgtggctgcactggatgggaccttccagagg
601 aagccatttggggccatcctgaacctggtgccgctggccgagagcgtggtgaagctgacg
661 gcggtgtgcatggagtgcttccgggaagccgcctataccaagaggctcggcacagagaag
721 gaggtcgaggtgattgggggagcagacaagtaccactccgtgtgtcggctctgctacttc
781 aagaaggcctcaggccagcctgccgggccggacaacaaagagaactgcccagtgccagga
841 aagccaggggaagccgtggctgccaggaagctctttgccccacagcagattctgcaatgg
901 atccagacatga
SEQ ID No. 24:
Human TK1 mutA75QV84E aa sequence
    1 MSCINLPTVL PGSPSKTRGQ IQVILGPMFS GKSTELMRRV RREQIAQYKC
  51 LVIKYAKDTR YSSSFCTHDR NTMEQLPACL LRDEAQEALG VAVIGIDEGQ
101 FFPDIVEFCE AMANAGKTVI VAALDGTFQR KPEGAILNLV PLAESVVKLT
151 AVCMECFREA AYTKRLGTEK EVEVIGGADK YHSVCRLCYF KKASGQPAGP
201 DNKENCPVPG RPGEAVAARK LFAPQQILQW IQT
SEQ ID No. 25:
Human TK1 mutL8081F nt sequence
                                                                  atgagctgcattaacctgcccactgtgctg
241 cccggctcccccagcaagacccgggggcagatccaggtgattctcgggccgatgttctca
301 ggaaaaagcacagagttgatgagacgcgtccgtcgcttccagattgctcagtacaagtgc
361 ctggtgatcaagtatgccaaagacactcgctacagcagcagcttctgcacacatgaccgg
421 aacaccatggaggcactgcccgcctgcttcttccgagacgaggcccaggaggccctgggc
481 gtggctgtcataggcatcgacgaggggcagtttttccctgacatcgtggagttctgcgag
541 gccatggccaacgccgggaagaccgtaattgtggctgcactggatgggaccttccagagg
601 aagccatttggggccatcctgaacctggtgccgatggccgagagcgtggtgaagctgacg
661 gcggtgtgcatggagtgcttccgggaagccgcctataccaagaggctcggcacagagaag
721 gaggtcgaggtgattgggggagcagacaagtaccactcagtgtgtcggctctgctacttc
781 aagaaggcctcaggccagcctgccgggccggacaacaaagagaactgcccagtgccagga
841 aagccaggggaagccgtggctgccaggaagctctttgccccacagcagattctgcaatgg
901 atccagacatga
SEQ ID No. 26:
Human TK1 mutL8081F aa sequence
    1 MSCINLPTVL PGSPSKTRGQ IQVILGPMFS GKSTELMRPV RRFQIAQYKC
  51 LVIKYAKDTR YSSSFCTHDR NTMEALPACF FRDVAQEALG VAVIGIDEGQ
101 FFPDIVEFCE AMANAGKTVI VAALDGTFQR KPFGAILNLV PLAESVVKLT
151 AVCMECFREA AYTKRLGTEK EVEVIGGADK YHSVCRLCYF KKASGQPAGP
201 DNKENCPVPG RPGEAVAARK LFAPQQILQW IQT
SEQ ID No. 27:
Human TK1 wt NM_003258 nt sequence (ORF)
                                                                  atgagctgcattaacctgcccactgtgctg
241 cctggctcccccagcaagacccgggggcagatccaggtgattctcgggccgatgttctca
301 ggaaaaagcacagagttgatgagacgcgtccgtcgcttccagattgctcagtacaagtgc
361 ctggtgatcaagtatgccaaagacactcgctacagcagcagcttctgcacacatgaccgg
421 aacaccatggaggcactgcccgcctgcctgctccgagacgtggcccaggaggccatgggc
481 gtggctgtcataggcatcgacgaggggcagtttttccctgacatcgtggagttctgcgag
541 gccatggccaacgccgggaagaccgtaattgtggctgcactggatgggaccttccagagg
601 aagccatttggggccatcctgaacctggtgccgctggccgagagcgtggtgaagctgacg
681 gcggtgtgcatggagtgcttccgggaagccgcctataccaagaggccggicacagagaag
721 gaggtcgaggtgattgggggagcagacaagtaccactccgtgtgtcggctctgctacttc
781 aagaaggcctcaggccagcctgccgggccggacaacaaagagaactgaccagtgccagga
841 aagccaggggaagccgtggctgccaggaagctctttgccccacagcagattctgcaatgc
901 agccctgccaactga
SEQ ID No. 28:
Human TK1 wt NM_003258 aa sequence
    1 MSCINLPTVL PGSPSKTRGQ IQVILGPMFS GKSTELMRRV RRFQIAQYKC
  51 LVIKYAKDTR YSSSFCTHDR NTMEALPACL LRDVAQEALG VAVIGIDEGQ
101 FFPDIVEFCE AMANAGKTVI VAALDGTFQR KPFGAILNLV PLAESVVKLT
151 AVCMECFREA AYTKRLGTEK EVEVIGGADK YHSVCRLCYF KKASGQPAGP
201 DNKENCPVPG KPGEAVAARK LFAPQQILQC SPAN
```

1. A nucleic acid for use in treating cancer, wherein said nucleic acid comprises a nucleotide sequence, wherein said nucleotide sequence encodes a mutant human thymidine kinase 1, wherein said mutant human thymidine kinase 1 comprises one or more amino acid substitutions at positions corresponding to positions 70 to 100 of wild-type human thymidine kinase 1.

2. A method of treating cancer, comprising administering a nucleic acid to a subject, wherein said nucleic acid comprises a nucleotide sequence, wherein said nucleotide sequence encodes a mutant human thymidine kinase 1, wherein said mutant human thymidine kinase 1 comprises one or more amino acid substitutions at positions corresponding to positions 70 to 100 of wild-type human thymidine kinase 1.

3. The nucleic acid for use of claim 1, or the method of claim 2, wherein the activity of said mutant human thymidine kinase 1 is increased compared to the activity of wildtype human thymidine kinase1.

4. The nucleic acid for use of claim 3, or the method of claim 3, wherein said activity is specific activity.

5. The nucleic acid for use of claim 3 or 4, or the method of claim 3 or 4, wherein said activity of said mutant human thymidine kinase 1 is at least 9-fold increased compared to the activity of wildtype human thymidine kinase 1.

6. The nucleic acid for use of claim 5, or the method of claim 5, wherein said mutant human thymidine kinase 1 comprises one or more amino acid substitutions at positions corresponding to positions 71 to 95 of wild-type human thymidine kinase 1.

7. The nucleic acid for use of claim 6, or the method of claim 6, wherein said mutant human thymidine kinase 1 comprises one or more amino acid substitutions at positions corresponding to one or more positions 73, 75, 83, 84, 90 and 95 of wild-type human thymidine kinase 1.

8. The nucleic acid for use of claim 7, or the method of claim 7, wherein said mutant human thymidine kinase 1 comprises one or two amino acid substitutions at positions corresponding to positions 84 and/or 90 of wild-type human thymidine kinase 1.

9. The nucleic acid for use of any one of claims 1 and 3 to 8, or the method of any one of claims 2 to 8, wherein said mutant human thymidine kinase 1 comprises one or more aliphatic, nonpolar, and neutral amino acid, one or more acid/amide, acidic polar and negatively charged amino acid and/or one or more acid/amide, polar and neutral amino acid at one or more positions corresponding to positions 70 to 100 of wild-type human thymidine kinase 1.

10. The nucleic acid for use of any one of claims 1 and 3 to 9, or the method of any one of claims 2 to 9, wherein said mutant human thymidine kinase 1 comprises one or more aliphatic, nonpolar, and neutral amino acid at one or more positions corresponding to positions 73, 75, 83, 90, and 95 of wild-type human thymidine kinase 1.

11. The nucleic acid for use of any one of claims 1 and 3 to 9, or the method of any one of claims 2 to 9, wherein said mutant human thymidine kinase 1 comprises one or more aliphatic, nonpolar, and neutral amino acid at one or more positions corresponding to positions 73, 83, 90, and 95 of wild-type human thymidine kinase 1.

12. The nucleic acid for use of any one of claims 1 and 3 to 9, or the method of any one of claims 2 to 9, wherein said mutant human thymidine kinase 1 comprises one or more aliphatic, nonpolar, and neutral amino acid at one or more positions corresponding to positions 75 of wild-type human thymidine kinase 1.

13. The nucleic acid for use of any one of claims 1 and 3 to 9, or the method of any one of claims 2 to 9, wherein said mutant human thymidine kinase 1 comprises one or more aliphatic, nonpolar, and neutral amino acid at one or more positions corresponding to position 90 of wild-type human thymidine kinase 1.

14. The nucleic acid for use of any one of claims 1 and 3 to 9, or the method of any one of claims 2 to 9, wherein said mutant human thymidine kinase 1 comprises one or more acid/amide, acidic polar and negatively charged amino acid at one or more positions corresponding to positions 75, 84 and 90 of wild-type human thymidine kinase 1.

15. The nucleic acid for use of any one of claims 1 and 3 to 9, or the method of any one of claims 2 to 9, wherein said mutant human thymidine kinase 1 comprises one or two acid/amide, acidic polar and negatively charged amino acid at one or two positions corresponding to positions 84 and 90 of wild-type human thymidine kinase 1.

16. The nucleic acid for use of any one of claims 1 and 3 to 9, or the method of any one of claims 2 to 9, wherein said mutant human thymidine kinase 1 comprises a acid/amide, polar and neutral amino acid at a position corresponding to position 75 of wild-type human thymidine kinase 1.

17. The nucleic acid for use of any one of claims 1 and 3 to 9, or the method of any one of claims 2 to 9,
a) wherein said mutant human thymidine kinase 1 comprises an aliphatic, nonpolar, neutral amino acid at a position corresponding to position 73 of wild-type human thymidine kinase 1;
b) wherein said mutant human thymidine kinase 1 comprises an aliphatic, nonpolar, neutral amino acid or an acid/amide, polar, neutral amino acid or an acid/amide, acidic polar, negatively charged amino acid at a position corresponding to position 75 of wild-type human thymidine kinase 1;
c) wherein said mutant human thymidine kinase 1 comprises an aliphatic, nonpolar, neutral amino acid at a position corresponding to position 83 of wild-type human thymidine kinase 1;
d) wherein said mutant human thymidine kinase 1 comprises an acid/amide, acidic polar, negatively charged amino acid at a position corresponding to position 84 of human wild-type thymidine kinase 1;
e) wherein said mutant human thymidine kinase 1 comprises an aliphatic, nonpolar, neutral amino acid or an acid/amide, acidic polar, negatively charged amino acid at a position corresponding to position 90 of wild-type human thymidine kinase 1; and/or
f) wherein said mutant human thymidine kinase 1 comprises an aliphatic, nonpolar, neutral amino acid at a position corresponding to position 95 of wild-type human thymidine kinase 1.

18. The nucleic acid for use of any one of claims 1 and 3 to 17, or the method of any one of claims 2 to 17, wherein said mutant human thymidine kinase 1 comprises
a) aliphatic, nonpolar, and neutral amino acids at positions corresponding to positions 73, 83, 90 and 95 of wild-type human thymidine kinase 1 and an acid/amide, acidic polar and negatively charged amino acid at a position corresponding to position 75 of wild-type human thymidine kinase 1;
b) aliphatic, nonpolar, and neutral amino acids at positions corresponding to positions 73, 83, and 90 of wild-type human thymidine kinase 1 and an acid/amide, acidic polar and negatively charged amino acid at a position corresponding to position 75 of wild-type human thymidine kinase 1;
c) an acid/amide, acidic polar and negatively charged amino acid at a position corresponding to position 84 of wild-type human thymidine kinase 1 and an aliphatic, nonpolar, and neutral amino acid at a position corresponding to position 90 of wild-type human thymidine kinase 1;
d) aliphatic, nonpolar, and neutral amino acids at positions corresponding to positions 73, 90 and 95 of wild-type human thymidine kinase 1 and an acid/amide, acidic polar and negatively charged amino acid at a position corresponding to position 75 of wild-type human thymidine kinase 1;
e) aliphatic, nonpolar, and neutral amino acids at positions corresponding to positions 73 and 90 of wild-type human thymidine kinase 1 and an acid/amide, acidic polar and negatively charged amino acid at a position corresponding to position 75 of wild-type human thymidine kinase 1;
f) an aliphatic, nonpolar, and neutral amino acid at a position corresponding to position 90 of wild-type human thymidine kinase 1;
g) acid/amide, acidic polar and negatively charged amino acids at positions corresponding to positions 75 and 90 of wild-type human thymidine kinase 1;
h) an acid/amide, acidic polar and negatively charged amino acid at a position corresponding to position 84 of wild-type human thymidine kinase 1 and an aliphatic, nonpolar, and neutral amino acid at position corresponding to position 90 of wild-type human thymidine kinase 1;
i) aliphatic, nonpolar, and neutral amino acids at positions corresponding to positions 75 and 90 of wild-type human thymidine kinase 1 and an acid/amide, acidic polar and negatively charged amino acid at a position corresponding to position 84 of wild-type human thymidine kinase 1;
j) an acid/amide, acidic polar and negatively charged amino acid at a position corresponding to position 90 of wild-type human thymidine kinase 1;
k) an aliphatic, nonpolar, and neutral amino acid at a position corresponding to position 90 of wild-type human thymidine kinase 1; or
l) an acid/amide, polar and neutral amino acid at a position corresponding to position 75 of wild-type human thymidine kinase 1 and an acid/amide, acidic polar and negatively charged amino acid at a position corresponding to position 84 of wild-type human thymidine kinase 1.

19. The nucleic acid for use of any one of claims 1 and 3 to 17, or the method of any one of claims 2 to 17, wherein said mutant thymidine kinase comprises
a) an aliphatic, nonpolar, and neutral amino acid at a position corresponding to position 90 of wild-type human thymidine kinase 1; or
b) an acid/amide, acidic polar and negatively charged amino acid at a position corresponding to position 84 of wild-type human thymidine kinase 1 and an aliphatic, nonpolar, and neutral amino acid at a position corresponding to position 90 of wild-type human thymidine kinase 1.

20. The nucleic acid for use of any one of claims 9 to 13 and 17 to 19, or the method of any one of claims 9 to 13 and 17 to 19, wherein said aliphatic, nonpolar, and neutral amino acid is one or more of alanine, glycine, valine, isoleucine, leucine and methionine.

21. The nucleic acid for use of any one of claims 9 to 11, 17a), 17c), 17e) and 17f), or the method of any one of claims 9 to 11, 17a), 17c), 17e) and 17f), wherein said aliphatic, nonpolar, and neutral amino acid is alanine.

22. The nucleic acid for use of any one of claims 9, 10, 12 and 17b), or the method of any one of claims 9, 10, 12 and 17b), wherein said aliphatic, nonpolar, and neutral amino acid is glycine.

23. The nucleic acid for use of any one of claims 9, 10, 13 and 17e), or the method of any one of claims 9, 10, 13 and 17e), wherein said aliphatic, nonpolar, and neutral amino acid is valine.

24. The nucleic acid for use of any one of claims 9, 14, 15, and 17 to 19, or the method of any one of claims 9, 14, 15, and 17 to 19, wherein said acid/amide, acidic polar and negatively charged amino acid is one or more of aspartic acid and glutamic acid.

25. The nucleic acid for use of any one of claims 9, 14, 17b), 17d), 17e), 18a), 18b), 18d), 18e), 18g), 18h) and 18j), or the method of any one of claims 9, 14, 17b), 17d), 17e), 18a), 18b), 18d), 18e), 18g), 18h) and 18j), wherein said acid/amide, acidic polar and negatively charged amino acid is aspartic acid.

26. The nucleic acid for use of any one of claims 9, 15, 17d), 17e), 18c), 18g), 18i) and 18l), or the method of any one of claims 9, 15, 17d), 17e), 18c), 18g), 18i) and 18l), wherein said acid/amide, acidic polar and negatively charged amino acid is glutamic acid.

27. The nucleic acid for use of any one of claims 9 and 16 to 18, or the method of any one of claims 9 and 16 to 18, wherein said acid/amide, polar and neutral amino acid is one or more of glutamine, asparagine.

28. The nucleic acid for use of any one of claims 9, 16, 17b), and 18l), or the method of any one of claims 9, 16, 17b), and 18l), wherein said acid/amide, polar and neutral amino acid is glutamine.

29. The nucleic acid for use of any one of claims 1 and 3 to 28, or the method of any one of claims 2 to 28, wherein said mutant human thymidine kinase 1 comprises
a) alanine at positions corresponding to positions 73, 83, 90 and 95 of wild-type human thymidine kinase 1 and aspartic acid at a position corresponding to position 75 of wild-type human thymidine kinase 1;
b) alanine at positions corresponding to positions 73, 83, and 90 of wild-type human thymidine kinase 1 and aspartic acid at a position corresponding to position 75 of wild-type human thymidine kinase 1;
c) glutamic acid at a position corresponding to position 84 of wild-type human thymidine kinase 1 and alanine at a position corresponding to position 90 of wild-type human thymidine kinase 1;
d) alanine at positions corresponding to positions 73, 90 and 95 of wild-type human thymidine kinase 1 and aspartic acid at a position corresponding to position 75 of wild-type human thymidine kinase 1;
e) alanine at positions corresponding to positions 73, and 90 of wild-type human thymidine kinase 1 and aspartic acid at a position corresponding to position 75 of wild-type human thymidine kinase 1;
f) alanine at a position corresponding to position 90 of wild-type human thymidine kinase 1;
g) aspartic acid at a position corresponding to position 75 of wild-type human thymidine kinase 1 and glutamic acid at a position corresponding to position 90 of wild-type human thymidine kinase 1;
h) aspartic acid at a position corresponding to position 84 of wild-type human thymidine kinase 1 and alanine at position corresponding to position 90 of wild-type human thymidine kinase 1;
i) glycine at a position corresponding to position 75 of wild-type human thymidine kinase 1, glutamic acid at a position corresponding to position 84 of wild-type human thymidine kinase 1 and alanine at a position corresponding to position 90 of wild-type human thymidine kinase 1;
j) aspartic acid at a position corresponding to position 90 of wild-type human thymidine kinase 1;
k) valine at a position corresponding to position 90 of wild-type human thymidine kinase 1; or
l) glutamine at a position corresponding to position 75 of wild-type human thymidine kinase 1 and glutamic acid at a position corresponding to position 84 of wild-type human thymidine kinase 1.

30. The nucleic acid for use of any one of claims 1 and 3 to 29, or the method of any one of claims 2 to 29, wherein said mutant human thymidine kinase 1 comprises
a) alanine at a position corresponding to position 90 of wild-type human thymidine kinase 1; or
b) aspartic acid amino acid at a position corresponding to position 84 of wild-type human thymidine kinase 1 and an alanine at a position corresponding to position 90 of wild-type human thymidine kinase 1.

31. The nucleic acid for use of any one of claims 1 and 3 to 30, or the method of any one of claims 2 to 30, wherein said human wild-type thymidine kinase 1 has an amino acid sequence shown in SEQ ID NO. 28.

32. The nucleic acid for use of any one of claims 1 and 3 to 31, or the method of any one of claims 2 to 31, wherein said nucleic acid is selected from the group consisting of:
a) a nucleic acid comprising a nucleotide sequence, wherein said nucleotide sequence encodes a mutant thymidine kinase, wherein said mutant human thymidine kinase 1 comprises an amino acid sequence as depicted in any one of SEQ ID NOs: 2, 4, 6, 8, 10, 12, 14, 16, 18, 20, 22 and 24;
b) a nucleic acid comprising a nucleotide sequence, wherein said nucleotide sequence encodes a mutant human thymidine kinase 1, and wherein said nucleotide sequence is depicted in SEQ ID NO: 1, 3, 5, 7, 9, 11, 13, 15, 17, 19, 21 and 23;
c) a nucleic acid hybridizing under stringent conditions to the complementary strand of the nucleic acid as defined in (a) or (b) and wherein said nucleic acid comprises a nucleotide sequence wherein said nucleotide sequence encodes a mutant human thymidine kinase 1;
d) a nucleic acid comprising a nucleotide sequence with at least 70% identity to the nucleotide sequence of the nucleic acids of any one of (a) to (c) and wherein said nucleotide sequence encodes a mutant human thymidine kinase 1; and
e) a nucleic acid comprising a nucleotide sequence which is degenerate as a result of the genetic code to the nucleotide sequence of a nucleic acid of any one of (a) to (d) and wherein said nucleotide sequence encodes a mutant human thymidine kinase 1.

33. The nucleic acid for use of any one of claims 1 and 3 to 31, or the method of any one of claims 2 to 31, wherein said nucleic acid is selected from the group consisting of:
a) a nucleic acid comprising a nucleotide sequence, wherein said nucleotide sequence encodes a mutant human thymidine kinase 1, wherein said mutant thymidine kinase comprises an amino acid sequence as depicted in any one of SEQ ID NOs: 10 and 12;
b) a nucleic acid comprising a nucleotide sequence, wherein said nucleotide sequence encodes a mutant human thymidine kinase 1, and wherein nucleotide sequence is depicted in SEQ ID NO: 9 and 11;
c) a nucleic acid hybridizing under stringent conditions to the complementary strand of the nucleic acid as defined in (a) or (b) and wherein said nucleic acid comprises a nucleotide sequence wherein said nucleotide sequence encodes a mutant human thymidine kinase 1;
d) a nucleic acid comprising a nucleotide sequence with at least 70% identity to the nucleotide sequence of the nucleic acids of any one of (a) to (c) and wherein said nucleotide sequence encodes a mutant human thymidine kinase 1; and
e) a nucleic acid comprising a nucleotide sequence which is degenerate as a result of the genetic code to the nucleotide sequence of a nucleic acid of any one of (a) to (d) and wherein said nucleotide sequence encodes a mutant human thymidine kinase 1.

34. Nucleic acid as defined in any one of claims 1 to 33.

35. Vector comprising the nucleic acid of claim 34.

36. The vector of claim 35, wherein said vector is a gene therapy vector.

37. The vector of claim 35 or 36, wherein said vector is an AAV vector, adenovirus vector, or a lentivirus vector.

38. Protein encoded by the nucleic acid of claim 34.

39. Composition comprising the nucleic acid of claim 34, a vector of any one of claims 35 to 37, or the protein of claim 38.

40. The composition of claim 39, wherein said composition is a pharmaceutical composition.

41. The nucleic acid of claim 34, the vector of any one of claims 35 to 37, the protein of claim 38, or the composition of claim 39 or 40, wherein said nucleic acid, said vector, said protein, or said composition is for use as a medicament.

42. The vector of any one of claims 35 to 37, the protein of claim 38, or the composition of claim 39 or 40, wherein said vector, said protein or said composition is for use in treating cancer.

43. The nucleic acid for use of any one of claims 1 and 3 to 33, or the method of any one of claims 2 to 33, the vector for use of claim 42, the protein for use of claim 42, or the composition for use of claim 42, wherein said treatment of cancer comprises administration of deoxythymidine.

44. The nucleic acid for use of any one of claims 1, 3 to 33 and 43, or the method of any one of claims 2 to 33 and 43, the vector for use of claim 42 or 43, the protein for use of claim 42 or 43, or the composition for use of claim 42 or 43, wherein said treatment of cancer comprises administration of additional chemotherapeutic agents, surgery and/or radiotherapy.

45. The nucleic acid for use of claim 44, or the method of claim 44, the vector for use of claim 44, the protein for use of claim 44, or the composition for use of claim 44, wherein said chemotherapeutic agents are Cytarabin (araC) and/or 5-Fluoruracil (5-FU).

46. The nucleic acid for use of any one of claims 1, 3 to 33, 43, 44 and 45, or the method of any one of claims 2 to 33, 43, 44 and 45, the vector for use of any one of claims 42 to 45, the protein for use of any one of claims 42 to 45, or the composition for use of any one of claims 42 to 45, wherein said cancer is a solid cancer.

47. The nucleic acid for use of any one of claims 1 and 3 to 33, 43, 44 and 45, or the method of any one of claims 2 to 33, 43, 44 and 45, the vector for use of any one of claims 42 to 45, the protein for use of any one of claims 42 to 45, or the composition for use of any one of claims 42 to 45, wherein said cancer is selected from the group consisting of cervical cancer, prostate carcinoma, ductal mammary carcinoma, melanoma, colon cancer, lung cancer, liver cancer, glioblastoma, and nerve cell cancer.
